(19) United States
(12) Patent Application Publication
(10) Pub. No.: US 2022/0185824 A1
(43) Pub. Date: Jun. 16, 2022

(54) PI-CONJUGATED COMPOUND, METHOD FOR PRODUCING PI-CONJUGATED COMPOUND, INK COMPOSITION, ORGANIC ELECTROLUMINESCENT ELEMENT MATERIAL, LIGHT EMITTING MATERIAL, CHARGE TRANSPORT MATERIAL, LIGHT EMITTING FILM AND ORGANIC ELECTROLUMINESCENT ELEMENT

(71) Applicant: Konica Minolta, Inc., Tokyo (JP)

(72) Inventors: Ryutaro SUGAWARA, Machida-shi, Tokyo (JP); Kazuma ODA, Hino-shi, Tokyo (JP); Hiroki TATSUMI, Toyohashi-shi, Aichi (JP)

(21) Appl. No.: 17/440,094

(22) PCT Filed: Feb. 14, 2020
(86) PCT No.: PCT/JP2020/005764

§ 371 Date: Sep. 16, 2021

(30) Foreign Application Priority Data
Mar. 18, 2019 (JP) 2019-050093

(51) Int. Cl. C07D 519/00; H01L 51/00; C07F 5/02; C07F 7/08

(52) U.S. Cl. CPC C07D 519/00; H01L 51/0067; H01L 51/0072; H01L 51/5056; H01L 51/0094; C07F 5/02; C07F 7/0816; H01L 51/008

(57) ABSTRACT

Disclosed is a π-conjugated compound that has a structure of the following Formula (1) and a HOMO level of −5.3 eV or higher,

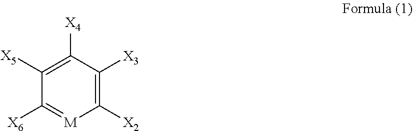

in the formula, M represents CX₁ or a nitrogen atom, X₁ to X₆ each represent an aromatic substituent or an aromatic heterocyclic group, in which at least four of X₁ to X₆ are electron-donating condensed aromatic ring substituents with 14 or more n electrons, and at least one of X₁ to X₆ is an electron-attracting substituent, and X₁ to X₆ may each independently further have a substituent.

### TECHNICAL FIELD

The present invention relates to a novel π-conjugated compound, a method for producing the π-conjugated compound, an ink composition, an organic electroluminescent element material, a light emitting material, a charge transport material, a light emitting film and an organic electroluminescent element. In more detail, the present invention relates to a π-conjugated compound or the like that has an improved hole injection property causing good element drive voltage and that can reduce aggregates such as exciplex excimers by the π plane-shielded structure.

### BACKGROUND

Organic electroluminescent (hereinafter referred to as “EL”) elements (also referred to as “organic EL elements”), which are based on electroluminescence of organic materials, have already been put into practice as a new generation of light emitting system that enables planar light emission. Organic EL elements have recently been applied to electronic displays and also to lighting devices. Thus, further development of organic EL elements is expected.

As an emission mode of an organic EL element, there are two types. One is “a phosphorescence emission type” in which light is emitted when a triplet excited state returns to a ground state, and another one is “a fluorescence emission type” in which light is emitted when a singlet excited state returns to a ground state.

When an electric filed is applied to an organic EL element, a hole and an electron are respectively injected from an anode and a cathode, they are recombined in a light emitting layer to produce an exciton. At this moment, a singlet excitor) and a triplet exciton are formed with a ratio of 25%:75%. Therefore, it is known that a phosphorescence emission type using a triplet exciton will produce theoretically high internal quantum efficiency compared with a fluorescence emission type.

However, in order to obtain high quantum efficiency in a phosphorescence emission type, it is required to use a complex compound having a rare metal of iridium or platinum in the center metal. This may induce an industrial problem of the amount of deposits or the cost of the rare metals in the future.

On the other hand, in recent years, new techniques relevant to a fluorescence emission type have been proposed to improve emission efficiency.

For example, Patent Document 1 discloses a technique which is focused on a phenomenon that a singlet exciton is generated by collision of two triplet excitors (Triplet-Triplet Annihilation (TTA), also referred to as Triplet-Triplet Fusion (TTF)), and which improves luminous efficiency of a fluorescent element by allowing the TTA phenomenon to occur effectively. Although this technique can increase power efficiency of a fluorescence emission material from two to three times larger than the power efficiency of a conventional fluorescent material, the emission efficiency in TTA is not as high as that of the aforementioned phosphorescent material due to a theoretical limitation, because the rate of conversion of the excited triplet energy level to the excited singlet energy remain to about 40%.

Recent studies have disclosed a fluorescent material that employs a thermally activated delayed fluorescent mechanism (hereinafter also abbreviated as “TADF”) that causes reverse intersystem crossing (hereinafter also abbreviated as “RISC”) from a triplet exciton to a singlet exciton. It has been also reported that such a material may be applicable to an organic EL element (for example, see Patent Document 2 and Non-patent Documents 1 to 2). By making use of this delayed fluorescence caused by the TADF mechanism, theoretically, it is possible to achieve an internal quantum efficiency of 100% in fluorescence emission, which is similar to the phosphorescent emission, in fluorescent emission by electric field excitation.

In order to make appear the TADF phenomenon, it is required that a reverse intersystem crossing from the triplet state, which is produced with an amount of 75% by an electric field excitation in an amount of 75% at room temperature or at an emission layer temperature on the emission device, to the singlet state should be taken place. Further, by the mechanism that the singlet exciton produced by the reverse intersystem crossing emits fluorescence in the same way as the singlet exciton produced with an amount of 25%, it is theoretically possible to realize 100% internal quantum efficiency. The reverse intersystem crossing requires small absolute value (ΔEST) of the difference between the lowest excited singlet energy level (S₁) and the lowest triplet excited singlet energy level (T₁).

Further, it is known that an addition of the third component (an assist-dopant material) which exhibits a TADF property into a light emitting layer composed of a host material and an emission material is effective to achieve high efficiency (see Non-patent document 3).

By producing 25% singlet excitons and 75% triplet excitons by electric field excitation on the assist-dopant material, the triplet excitons may produce singlet excitons accompanied with reverse intersystem crossing (RISC). The energy of the singlet excitations transfers to the emission material by fluorescence resonance energy transfer (hereinafter also abbreviated as “FRET”), and the transferred energy allows the emission material to emit light. Therefore, it is possible to cause the emission material to emit light using 100% of the exciton energy theoretically, and high luminous efficiency is expressed.

As described above, a variety of studies has been made to improve the light emission efficiency of conventional organic EL elements, and remarkable results have been obtained. However, there are many technical problems in practical use, and one of the major problems is to achieve both low voltage and long lifetime of an element.

In particular, since the excitons have large energy in a blue light emitting element, a lower energy component causes quenching. It can be said that it is even more difficult to extend the lifetime of a blue emitting element than a green or red light emitting element.

The interface resistance due to the energy barrier exists at each interface of the layers of the organic EL element. Therefore, the lower the interface resistance, the lower the drive voltage of an organic EL element.

For example, the hole injection properly from a hole transport layer to a light emitting layer is considered. The smaller HOMO level difference between the hole transport layer and the light emitting layer means the lower hole injection barrier, which results in the lower drive voltage of the organic EL element. Since the hole transport layer is often composed of a compound having a shallow (high) HOMO level such as an arylamine compound, a light emitting material having the same HOMO level as the hole transport layer can reduce the hole injection barrier. Examples of such light emitting materials include an arylamine structure and a carbazole structure further substituted with an electron donating group.

For example, Non-Patent Document 3 discloses an organic EL element in which TAPC (4,4′-Cyclohexylidenebis[N,N-bis(4-methylphenyl)bentenemine]), which is an arylamine compound, is used as a hole transport material, and DACT-II (9-[4-(4,6-diphenyl-1,3,5-triazin-2-yl)phenyl]-N,N,N′,N′-tetraphenyl-9H-carbazole-3,6-diamine), which is a TADF material composed of carbazole substituted with an electron-donating diphenylamino group, is used as a light emitting material.

While it is described that the organic EL element exhibits high efficiency, the drive voltage and the drive lifetime thereof are not mentioned

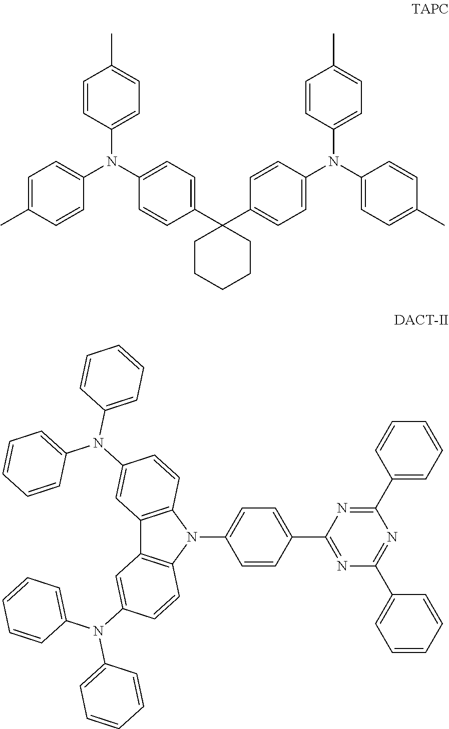

Non-Patent Document 4 discloses a technique of extending the lifetime of an organic EL element by using a TADF material. Specifically, the document discloses a technique using, 3Cz3DPhCzBN (2,4,6-tris(9H-carbazole-9-yl)-3,5-bis(3,6-diphenylcarbazole-9-yl)bentonirile), which is a material focusing on the delayed fluorescence lifetime of a light-emitting material. However, low voltage drive is not mentioned.

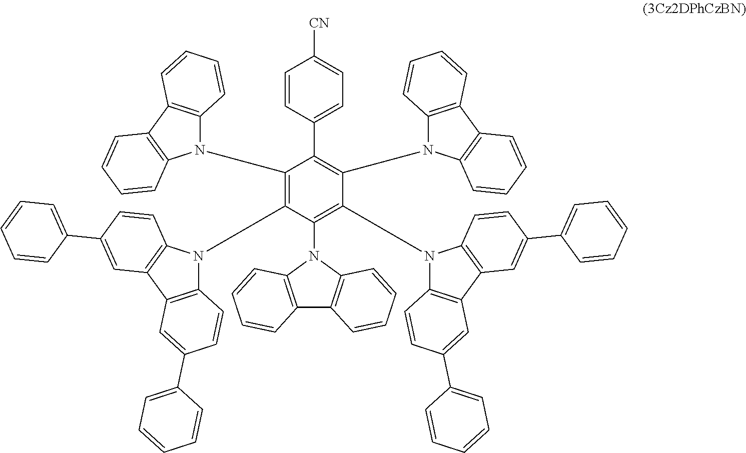 [SMILES: N#Cc1ccc(-c2c(-n3c4ccccc4c4ccccc43)c(-n3c4ccc(-c5ccccc5)cc4c4cc(-c5ccccc5)ccc43)c(-n3c4ccccc4c4ccccc43)c(-n3c4ccc(-c5ccccc5)cc4c4cc(-c5ccccc5)ccc43)c2-n2c3ccccc3c3ccccc32)cc1]

The present inventor examined the organic EL elements using the materials shown in Patent Documents 1 and 2, and Non-Patent Documents 1 to 4. However, the elements did not have sufficiently satisfactory drive voltage and drive lifetime.

### CITATION LIST

### Patent Documents

Patent document 1: WO 2010/134350A

Patent Document 1: JP 2013-116975A

### Non-Patent Documents

Non-Patent Document 1: H. Uoyama, et al., Nature, 2012, 492, 234-238

Non-Patent Document 2: Q. Zhang et al., Nature, Photonics, 2014, 8, 326-332.

Non-Patent Document 3: H. Kaji, et al., Nature Communications, 2015, 6, 8476

Non-Patent Document 4: H. Noda et al., Science Advances, 2018, 4, eaao6910

### SUMMARY OF INVENTION

### Technical Problem

The present invention has been made in view of the above-described problems and circumstances, and an object thereof is to provide a π-conjugated compound that can achieve a low voltage drive and a long lifetime and to provide a method for producing the same. Further, another object is to provide an ink composition containing the π-conjugated compound, an organic electroluminescent element material, a light-emitting material, a charge transport material, a light-emitting thin film, and an organic electroluminescent element.

### Solution to Problem

In order to solve the above-described problems, the present inventors studied the cause of the problems. In the process thereof, the inventors found that the hole injection property and the transportability are improved by employing a low-HOMO level structure, which is used for a hole transport material, to a donor moiety of a TADF material, and the drive voltage is thereby decreased. Furthermore, the inventors found that a structure shielding the π plane of the donor moiety can reduce formation of aggregates, and thereby suppress a decrease of the drive lifetime due to aggregates. Thus, the present invention has been made. That is, the above-described problems relating to the present invention are solved by the following means.

1. A π-conjugated compound that has a structure of the following Formula (1) and a HOMO level of −5.3 eV or higher,

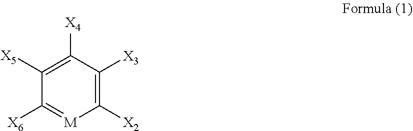

in the formula, M represents CX₁ or a nitrogen atom, X₁ to X₆ each represent an aromatic substituent or an aromatic heterocyclic group, in which at least four of X₁ to X₆ are electron-donating condensed aromatic ring substituents with 14 or more π electrons, and at least one of X₁ to X₆ is an electron-attracting substituent, and X₁ to X₆ may each independently further have a substituent.

2. The π-conjugated compound according to item 1, wherein in Formula (4), at least one of the electron-donating condensed aromatic substituents has a structure of any of the following (a) to (c),

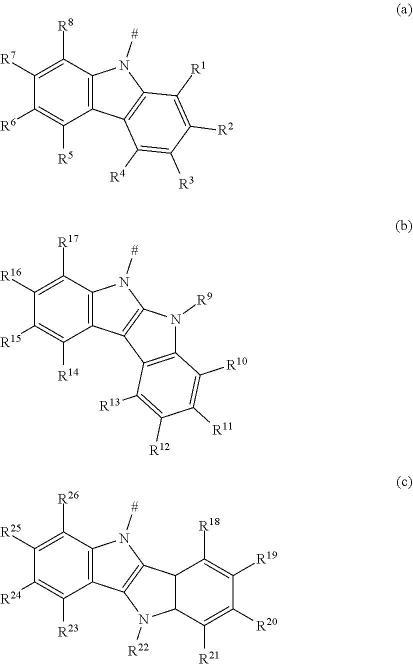

in Formulae (a) to (c), R¹ to R²⁶ each independently represent a hydrogen atom or a substituent, “#” represents a substitution position to Formula (1), and adjacent substituents may be combined with each other to form a ring structure.

3. The π-conjugated compound according to item 1 or 2, wherein the π-conjugated compound having the structure of Formula (1) has a structure of the following Formula (2),

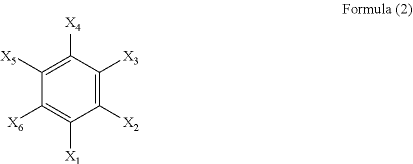

in the formula, X₁ to X₆ each represent an aromatic substituent or an aromatic heterocyclic group, in which at least four of X₁ to X₆ are electron-donating condensed aromatic ring substituents with 14 or more π electrons, and at least one of X₁ to X₆ is an electron-attracting substituent, and X₁ to X₆ may each independently further have a substituent.

4. The π-conjugated compound according to any one of items 1 to 3, wherein in Formula (1) or (2), the HOMO level is 5.0 eV or more.

5. The π-conjugated compound according to any one of items 1 to 4, wherein Formula (1) or (2), at least one of X₁ to X₆ has a substituent having a structure of the following Formula (3),

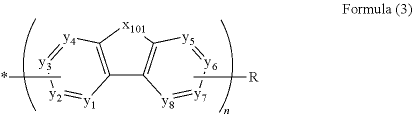

in the formula, a symbol “*” represents a binding position to any of X₁ to X₆ of Formula (1) or (2), X₁₀₁ represents NR₁₀₁, an oxygen atom, a sulfur atom, a sulfinyl group, a sulfonyl group, CR₁₀₂R₁₀₃ or SiR₁₀₄R₁₀₅, y₁ to y₈ each independently represent CR₁₀₆ or a nitrogen atom, R₁₀₁ to R₁₀₆ each independently represent a hydrogen atom or a substituent, in which R₁₀₁ to R₁₀₆ may be combined with each other to form a ring, n represents an integer of 1 to 4, and R represents a substituent.

6. The π-conjugated compound according to any one of items 1 to 5, wherein an absolute value ΔEst of an energy difference between a lowest excited singlet level and a lowest excited triplet level is 0.50 eV or less.

7. A method for producing the π-conjugated compound according to any one of items 1 to 6,

wherein each of the substituents X1 to X6 is introduced to a benzene ring by a nucleophilic substitution reaction.

8. An ink composition containing the π-conjugated compound according to any one of items 1 to 6.

9. An electroluminescent element material containing the π-conjugated compound according to any one of items 1 to 6.

10. A light emitting material containing the π-conjugated compound according to any one of items 1 to 6,

wherein the π-conjugated compound emits fluorescence.

11. The light emitting material according to items 10, wherein the π-conjugated compound emits delayed fluorescence.

12. A charge transport material containing the π-conjugated compound according to any one of items 1 to 6,

wherein the π-conjugated compound emits fluorescence.

13. The charge transport material according to items 12, wherein the π-conjugated compound emits delayed fluorescence.

14. A light emitting film containing the π-conjugated compound according to any one of items 1 to 6.

15. An organic electroluminescent element comprising a pair of electrodes and one or more light-emitting layers,

wherein at least one of the light-emitting layers contains the π-conjugated compound according to any one of items 1 to 6.

### Advantageous Effects of Invention

By the above-described solutions, provided are a π-conjugated compound that can achieve a low voltage drive and a long lifetime, and a method for producing the same. Further, it is possible to provide an ink composition containing the π-conjugated compound, an organic electroluminescent element material, a light-emitting material, a charge transport material, a light-emitting film, and an organic electroluminescent element.

The expression mechanism or action mechanism of the effect of the present invention is not clarified, but is inferred as follows.

The DACT-II disclosed in Non-Patent Document 3 is considered to have good hole injection property and good transportability since it has a shallow HOMO structure (having high electron donating property). However, since the π plane of an electron donating group or the electron-attracting group is not sterically shielded, a combined use with a material haying high electron-accepting property such as a TADF material or an electron transporting material may cause formation of aggregates such as eximers or exciplexes. Such aggregates emit light with lower energy than the dopant used and act as a quencher, which causes a decrease of the drive lifetime.

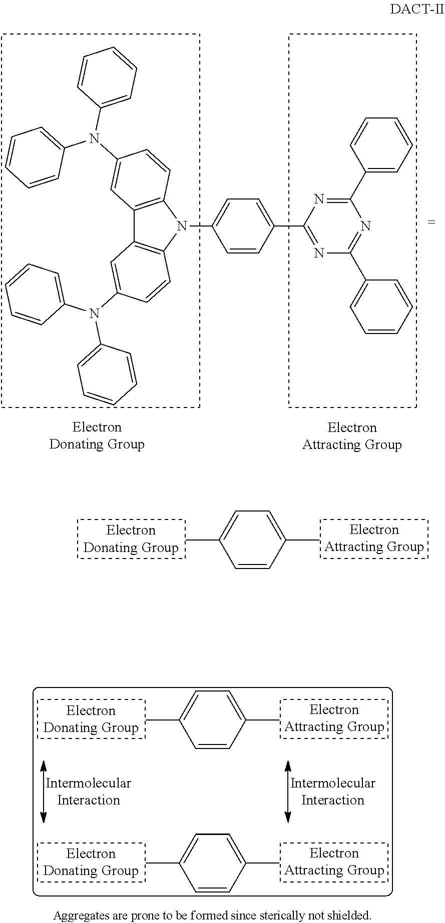

In the compound of the present invention, a shallow HOMO structure, which is used for hole transport materials, is employed in an electron donating group. This improves the hole injection property and the transportability.

Further, the compound of the present invention is characterized in that a benzene ring or a pyridine ring is substituted with at least four electron-donating condensed aromatic substituents each with 14 or more π electrons and at least one electron-attracting substituent. In such a compound, the π planes of the electron-attracting groups and the electron attracting group are sterically shielded by adjacent substituents, which suppresses formation of aggregates. That is, quenching due to aggregates is less likely to occur, and a long-life organic EL element can be achieved.

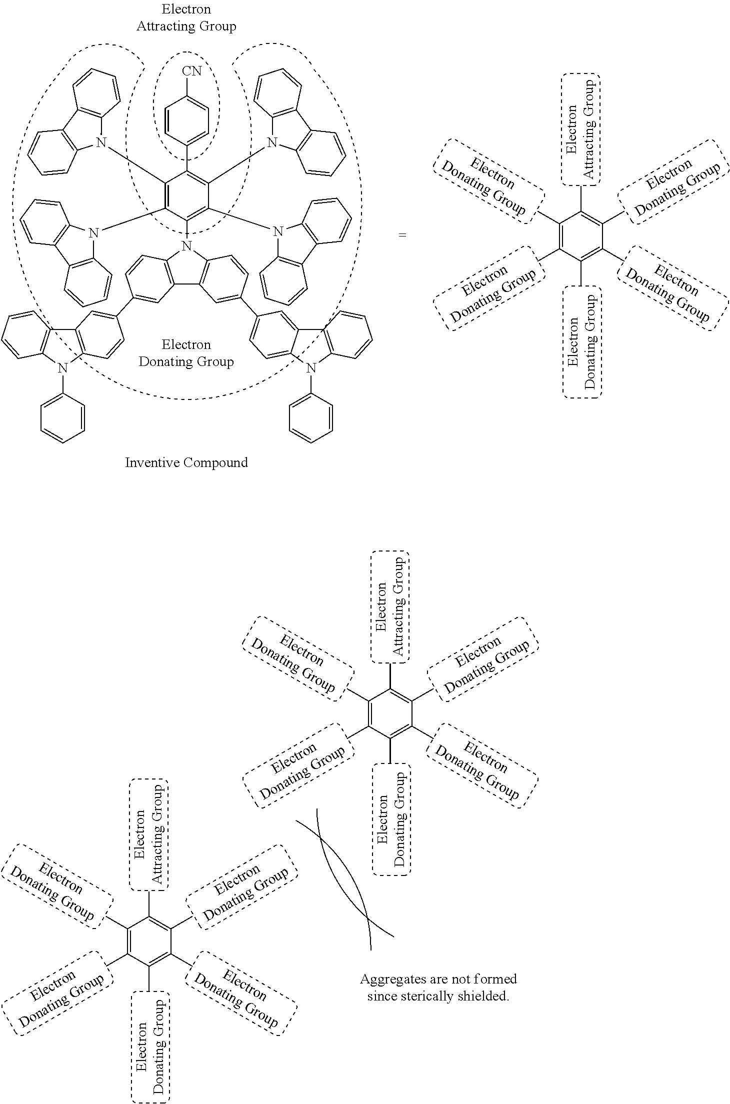

### BRIEF DESCRIPTION OF DRAWINGS

FIG. 1 is a schematic diagram showing an example of a method of manufacturing an organic EL element by inkjet printing.

FIG. 2A is a schematic external view of an inkjet head applicable to inkjet printing, showing an exemplary configuration thereof.

FIG. 2B is a schematic external view of an inkjet head applicable to inkjet printing, showing an exemplary configuration thereof.

FIG. 3 is a schematic diagram of a lighting device.

FIG. 4 is a schematic diagram of a lighting device.

### DESCRIPTION OF EMBODIMENTS

The π-conjugated compound of the present invention is characterized by having the structure of Formula (1) and a HOMO level of −5.3 eV or more.

This feature is a technical feature common to or corresponding to each of the following embodiments.

As an embodiment of the present invention, it is preferable that at least one of the electron donating condensed aromatic substituents has any one of the above-described structures (a) to (c) in Formula (1). This is because formation of aggregates can be further reduced and the lifetime can be further extended.

It is preferable that the π-conjugated compound having the structure of Formula (1) has the structure of Formula (2). This is because formation of aggregates can be further reduced and the lifetime can be further extended.

In Formula (1) or (2), it is preferable that the HOMO level is equal to or greater than −5.0 eV. This is because the shallow HOMO level improves the hole injection property and the transportability.

In Formula (1) or (2), it is preferable that at least one of X₁ to X₅ has a substituent having the structure of Formula (3). This is because formation of aggregates can be further reduced and the lifetime can be further extended.

It is preferable that the absolute value ΔEst of the energy difference between the lowest excited singlet level and the lowest excited triplet level is 0.50 eV or less, because the intersystem crossing from the lowest excited triplet energy level to the lowest excited singlet energy level, which was originally forbidden, is apt to occur and TADF becomes high.

In the method of producing the π-conjugated compound of the present invention, each of the substituents X₁ to X₆ are introduced to the benzene ring by a nucleophilic substitution reaction. This allows high yield production with a reduced amount of by-products.

The π-conjugated compound of the present invention is suitably used for an ink composition, an organic electroluminescent element material, and a light-emitting film.

Further, the π-conjugated compound of the present invention is suitably used as a light emitting material and a charge transporting material, and the π-conjugated compound emits fluorescence. In particular, it is preferable that the π-conjugated compound emits delayed fluorescence.

The organic electroluminescent element of the present invention includes at least a pair of electrodes and one or more light-emitting layers, wherein at least one of the light-emitting layers contains the π-conjugated compound. This allows for the low voltage drive and the long lifetime.

Hereinafter, the present invention and the components thereof, and aspects and embodiments of the present invention will be described. In the present description, when two numerical values are used to indicate a range of value before and after “to”, these values are included in the range as the lowest limit value and the upper limit value.

[π-Conjugated Compound of the Present Invention]

The π-conjugated compound of the present invention has the structure of the following Formula (1) and a HOMO level of −5.3 eV or more. It is preferable that the HOMO level is −5.0 eV or more. This is because the hole injection property and the transportability are further improved.

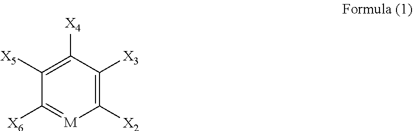

In the formula, M represents CX₁ or a nitrogen atom. X₁ to X₆ represent an aromatic substituent or an aromatic heterocyclic group, in which at least four of them are electron-donating condensed aromatic ring substituents with 14 or more π electrons, and at least one of them is an electron-attracting substituent. X₁ to X₆ may each independently further have a substituent.

Examples of the substituents X₁ to X₆ in Formula (1) include: an aromatic hydrocarbon ring group (also referred to as an aromatic carbon ring group or an aryl group, for example, groups derived from a phenyl ring, a biphenyl ring, a naphthalene ring, an angelic ring, an anthracene ring, a phenanthrene ring, a pyrene ring, a chrysene ring, a naphthacene ring, a triphenylene ring, an o-terphenyl ring, a m-terphenyl ring, a p-terphenyl ring, an acenaphthene ring, a coronene ring, an inden ring, a fluorene ring, a fluoranthene ring, a naphthalene ring, a pentacene ring, a perylene ring, a pentaphene ring, a picene ring, a pyrene ring, a pyranthrene ring, an anthraanthrene ring, and a tetraline ring); an aromatic heterocyclic group (for example, groups derived from a furan ring, a dibenzofuran ring, a thiophene ring, a dibenzothiophene ring, an oxazole ring, a pyrrole ring, a pyridine ring, a pyridazine ring, a pyrimidine ring, a pyrazine ring, a triazine ring, a benzimidazole ring, an oxadiazole ring, a triazole ring, an imidazole ring, a pyrazole ring, a thiazole ring, an indole ring, an indazole ring, a benzimidazole ring, a benzothiazole ring, a benzoxazole ring, a quinoxaline ring, a quinazoline ring, a cinnoline ring, a quinoline ring, an isoquinoline ring, a phthalazine ring, a naphthylidine ring, a dibenzoborol ring, a carbazole ring, carboline ring, a diazacarbazole ring (indicating a ring structure in which one of the carbon atoms constituting the carboline ring is further replaced with a nitrogen atom); an indroindole ring, an indolocarbazole ring, a diindrocarbazole ring, a 9,10-dihydroacrdine ring, a 5,10-dihydrophenazine ring, a 5,10-dihydrophenazacillin ring, a phenoxazine ring, a phenothiazine ring, a benzocarbazole ring and a dibenzocarbazole ring.

Examples of substituents that the substituents X₁ to X₆ can have include: an alkyl group (for example, a methyl group, an ethyl group, a propyl group, an isopropyl group, a tert-butyl group, a pentyl group, a hexyl group, an octyl group, a dodecyl group, a tridecyl group, a tetradecyl group, and a pentadecyl group); a cycloalkyl group (for example, a cyclopentyl group, a cyclohexyl group); an alkenyl group (for example, a vinyl group and an allyl group); an alkynyl group (for example, an ethynyl group and a propargyl group); an aromatic hydrocarbon group (for example, a phenyl group, a p-chlorophenyl group, a mesityl group, a tolyl group, a xylyl group, a naphthyl group, an anthryl group, an azulenyl group, an acenaphthenyl group, a fluolenyl group, a phenanthryl group, an indenyl group, a pyrenyl group, a biphenyl group); an aromatic heterocyclic group (for example, pyridyl group, a pyrimidinyl group, a furyl group, a pyrrolyl group, an imidazolyl group, a benzoimidazolyl group, a pyrazolyl group, a pyrazinyl group, triazolyl group (for example, a 1,2,4-triazol-1-yl group, a 1,2,3-triazol-1-yl group), a pyrazorotriazolyl group, an oxazolyl group, benzoxazolyl group, a thiazolyl group, an isooxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, a carbolinyl group, a diazacarbazolyl group (a carbolinyl with one of the carbon atoms of the carbo ring substituted with a nitrogen atom), a quinoxalinyl group, a pyridazinyl group, a triazinyl group, a quinazolinyl group, a phthalazinyl group); a heterocyclic group (for example, a pyrrolidyl group, an imidazolidyl group, a morpholyl group, and an oxazolidyl group); an alkoxy group (for example, a methoxy group, an ethoxy group, a propyloxy group, a pentyloxy group, an hexyloxy group, an octyloxy group, and a dodecyloxy group); a cycloalkoxy group (for example, a cyclopentyloxy group and a cyclohexyloxy group); an aryloxy group (for example, a phenoxy group and a napthyloxy group); an alkylthio group (for example, a methylthio group, an ethylthio group, a propylthio group, a pentylthio group, hexylthio group, an octylthio group, and a dodecylthio group); a cycloalkylthio group (for example, a cyclopentylthio group and a cyclohexylthio group); an arylthio group (for example, a phenylthio group and a naphthylthio group); an alkoxycarbonyl group (for example, a methyloxycarbonyl group, an ethyloxycarbonyl group, a butyloxycarbonyl group, an octyloxycarbonyl group, and a dodecyloxycarbonyl group); an aryloxycarbonyl group (for example, a phenyloxycarbonyl group and a naphthyloxycarbonyl group); a sulfamoyl group (for example, an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, and a 2-pyridylaminosulfonyl group); an acyl group (for example, an acetyl group, an ethyl carbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, and a pyridylcarbonyl group); an acyloxy group (for example, an acetyloxy group, an ethylcarbonyloxy group, a butylcarbonyloxy group, an octylcarbonyloxy group, a dodecylcarbonyloxy group, and a phenylcarbonyloxy group); an amino group (for example, a methylcarbonylamino group, an ethylcarbonylamino group, a dimethylcarbonylamino group, a propylcarbonylamino group, a pentylcarbonylamino group, a cyclohexylcarbonylamino group, a 2-ethyhexylcarbonylamino group, an octylcarbonylamino group, a dodecylcarbonylamino group, a phenylcarbonylamino group, and a naphthylcarbonylamino group); a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethymexylaminocarbonyl group, a dodecylaminocarbonyl group, a phenylaminocarbonyl group, a naphthylaminocarbonyl group, and a 2-pyridylaminocarbonyl group); a ureido group (for example, a methylureido group, an ethylureido group, a pentylureido group, a cyclohexylureido group, an octylureido group, a dodecylureido group, a phenylureido group, a naphthylureido group, and a 2-pyridylaminoureido group); a sulfinyl group (for example, a methylsulfinyl group, an ethylsufinyl group, a butylsulfinyl group, a cyclohexylsulfinyl group, a 2-ethylhexylsulfinyl group, a dodecylsulfinyl group, a phenylsulfinyl group, a naphthysulfinyl group, and a 2-pyridylsulfinyl group); an alkylsulfonyl group (for example, a methylsulfonyl group, an ethylsulfonyl group, a butylsulfinyl group, a cyclohexylsulfonyl group, a 2-ethylhexylsulfonyl group, and a dodecylsulfonyl group); an arylsulfonyl group or a heteroarylsulfonyl group (for example, a phenylsulfonyl group, a naphthylsulfonyl group, and a 2-pyridylsulfonyl group); an amino group (for example, an amino group, an ethylamino group, a dimethylamino group, a diphenylamino group, a diisopropylamino group, ditatebutyl group, a cyclohexylamino group, a butylamino group, a cyclopentylamino group, a dodecylamino group, an anilino group, a naphthylamino group, and a 2-pyridylamino group); a halogen atom (for example, a fluorine atom, a chlorine atom and a bromine atom); a fluorinated hydrocarbon group (for example, a fluoromethyl group, trifluoromethyl group, a pentafluoroethyl group and a pentafluorophenyl group); a cyano group; a nitro group; a hydroxy group; a mercapto group; a silyl group (for example, a trimethylsilyl group, a triisopropylsilyl group, a triphenylsilyl group, and a phenyldiethylsilyl group) and a phosphono group.

Examples of the electron-donating condensed aromatic ring substituents with 14 or more π electrons, which are represented by X₁ to X₆ in Formula (1), include a carbazole ring, an indoloindole ring, an indolocarbazole ring, a diindolocarbazole ring, a 9,10-dihydroacridin ring, a 5,10-dihydrophenazine ring, a 5,10-dihydrophenazacillin ring, a phenoxazine ring, a phenothiazine ring, a benzocarbazole ring, a dibenzocarbazole ring, an azacarbazole ring, and a diazacarbazole ring.

The molecular weight of the electron-donating condensed aromatic ring substituents is preferably 150 or more.

The electron-donating condensed aromatic ring substituents having a molecular weight of 150 or more are likely to release an electron and can have high electron donating property since a substituent that enhances the electron-donating property for an unpaired electron on the electron-donating condensed aromatic ring substituents or a nitrogen atom is introduced. The upper limit of the molecular weight of the electron-donating condensed aromatic ring substituents is, for example, 1000. The molecular weight of the electron-donating condensed aromatic ring substituents can be determined as a formula weight based on the composition formula thereof.

Examples of the electron-attracting substituent of X₁ to X₆ in Formula (1) includes an aromatic hydrocarbon ring group substituted with an electron-attracting group and a substituted or unsubstituted electron-attracting heterocyclic group.

Examples of the aromatic ring group of the above-described aromatic hydrocarbon ring group substituted with an electron-attracting group include a benzene ring and a naphthalene ring.

Examples of the electron-attracting group that the aromatic hydrocarbon ring group can have include an fluorine atom, a cyano group, a fluorine-substituted or unsubstituted alkyl group, a substituted or unsubstituted sulfonyl group, a substituted or unsubstituted boryl group, and a substituted or unsubstituted electron-attracting heterocyclic group.

The substituted or unsubstituted electron-attracting heterocyclic group is preferably a group that is derived from an electron-attracting aromatic heterocyclic ring with 3 to 24 carbon atoms.

Examples of such electron-attracting aromatic heterocyclic rings include a dibenzothiophene oxide ring, a dibenzothiophene dioxide ring, a pyridine ring, a pyridazine ring, pyrimidine ring, a pyrazine ring, a triazine ring, a quinoline ring, an isoquinoline ring, a quinazoline ring, a cinnoline ring, a quinoxalin ring, a phthalazine ring, a pteridin ring, a phenanthridin ring, a phenanthrolin ring, a dibenzofuran ring, an azadibenzofuran ring, a diazadibenzofuran ring, a dibenzosilol ring, a dibenzoborol ring, a dibenzophosphor oxide ring, and the like. Among them, preferred are a pyridine ring, a pyrimidine ring, a pyrazine ring, a triazine ring, a quinoline ring, an isoquinoline ring, a quinazoline ring, a quinoxaline ring, a phenanthridin ring, a phenanthroline ring, a dibenzofuran ring, an azadibenzofuran ring, and a diazadibenzofuran ring. More preferred are a nitrogen-containing aromatic heterocyclic ring, and yet more preferred are a nitrogen-containing aromatic six-membered ring, an azadibenzofuran ring, and a diazadibenzofuran ring.

The electron-attracting aromatic heterocyclic ring may be a combination of two or more of the same or different aromatic heterocyclic rings as described above that are coupled with each other.

Examples of substituents that the electron-attracting heterocyclic group can have include a deuterium atom., a fluorine atom, a cyano group, an alkyl group optionally substituted with fluorine, an aromatic hydrocarbon ring group optionally substituted with a fluorine-substituted or unsubstituted alkyl group, and an aromatic hydrocarbon group optionally substituted with a fluorine. Preferred are a fluorine atom, a cyano group, an alkyl group optionally substituted with a fluorine, and an aromatic hydrocarbon ring group optionally substituted with fluorine.

The molecular weight of the electron-attracting substituent can be about 69 to 1000. The molecular weight of the electron-attracting substituent can be determined by the same method for the molecular weight of the electron-donating condensed aromatic ring substituent.

It is preferable that at least one of the electron-donating condensed aromatic substituents has a structure of any of the following (a) to (c). This is because formation of aggregates can be further suppressed, and the lifetime can be extended.

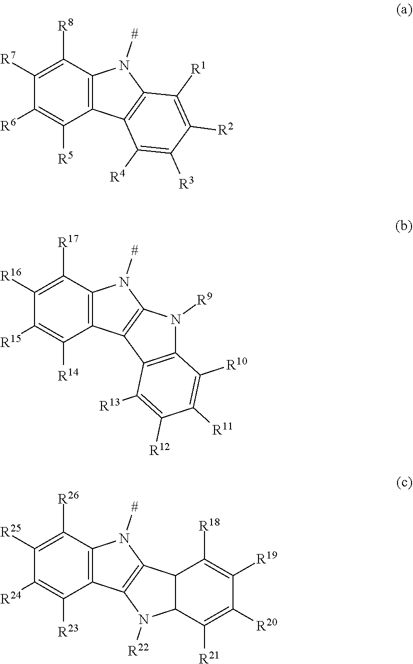

In Formulae (a) to (c), R¹ to R²⁶ each independently represent a hydrogen atom or a substituent. “#” represents a substitution position to Formula (1), and adjacent substituents may be bonded to each other to form a cyclic structure.

The number of substituents in Formulae (a) to (c) is not particularly limited. When there are two or more substituents, the substituents may be the same or different.

For the description and preferable range of the substituents, reference can be made to the description and preferable range of the substituents that the above-described substituents X₁ to X₆ can have.

Adjacent substituents may be combined to each other to form a cyclic structure.

Such a cyclic structure may be an aromatic ring or an alicyclic ring, contain a hetero atom, or be a condensed ring having two or more rings.

The hetero atom is preferably selected from the group consisting of a nitrogen atom, an oxygen atom and a sulfur atom. Examples of the cyclic structure to be formed include a benzene ring, a naphthalene ring, a pyridine ring, a pyridazine ring, pyrimidine ring, a pyrazine ring, a pyrrol ring, an imidazole ring, a pyrazole ring, a triazole ring, an imidazoline ring, an oxazole ring, an isooxazole ring, and a thiazole ring, an isothiazole ring, a cyclohexaziene ring, cyclohexene ring, a cyclopentaene ring, a cycloheptatriene ring, a cycloheptadiene ring, a cycloheptaene ring, a carbazole ring, and a dibenzofuran ring.

It is preferred that the π-conjugated compound having the structure of Formula (1) has the structure of the following Formula (2). This is because formation of aggregates can be further suppressed, and the lifetime can be extended.

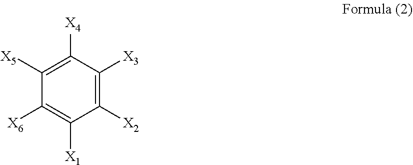

In the formula, X₁ to X₆ each represent an aromatic substituent or an aromatic heterocyclic group, in which at least four of them are electron-donating condensed aromatic ring substituents with 14 or more π electrons, and at least one of them is an electron-attracting substituent. In addition, X₁ to X₆ may each independently further have a substituent.

Examples of the aromatic substituents or aromatic heterocyclic groups represented by X₁ to X₆ in Formula (2) are the same as those for X₁ to X₆ in Formula (1).

In Formula (1) or (2), it is preferred that at least one of X₁ to X₆ has a substituent having the structure of the following Formula (3). This is because formation of aggregates can be further suppressed, and the lifetime can be extended.

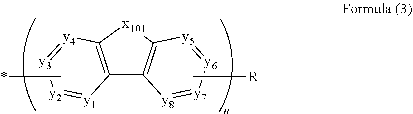

In the formula, the symbol “*” represents the binding position to any of X₁ to X₆ of Formula (1) or Formula (2). X ₁₀₁ represents NR₁₀₁, an oxygen atom, a sulfur atom, a sulfinyl group, a sulfonyl group, CR₁₀₂R₁₀₃ or SiR₁₀₄R₁₀₅. y₁ to y₈ each independently represents CR₁₀₆ or a nitrogen atom. R₁₀₁ to R₁₀₆ each independently represent a hydrogen atom or a substituent, and R₁₀₁ to R₁₀₅ may be combined to each other to form a ring. n represents an integer of 1 to 4. R represents a substituent.

R₁₀₁ to R₁₀₆ of Formula (3) each independently represent a hydrogen atom or a substituent. The substituent can be included in an extent that does not inhibit the functions used in the present invention. For example, even when a substituent is inevitably introduced during the synthetic scheme, compounds that have such a substituent but exhibit the advantageous effects of the present invention are included in the present invention.

Examples of the substituents represented by R₁₀₁ to R₁₀₆ include a linear or branched alkyl group (for example, a methyl group, an ethyl group, a propyl group, an isopropyl group, a t-butyl group, a pentyl group, a hexyl group, an octyl group, a dodecyl group, a tridecyl group, a tetradecyl group, a pentadecyl group, etc.), an alkenyl group (for example, a vinyl group, an allyl group, etc.), an alkynyl group (for example, an ethynyl group, a propargyl group, etc.), an aromatic hydrocarbon ring group (also referred to as an aromatic carbocyclic group or an aryl group, for example, groups derived from a phenyl ring, a biphenyl ring, a naphthalene ring, an azulene ring, an anthracene ring, a phenanthrene ring, a pyrene ring, a chrysene ring, a naphthacene ring, a triphenylene ring, an o-terphenyl ring, a m-terphenyl ring, a p-terphenyl ring, an acenaphthene ring, a coronene ring, an inden ring, a fluorene ring, a fluoranthene ring, a naphthalene ring, a pentacene ring, a perylene ring, a pentaphene ring, a picene ring, a pyrene ring, a pyranthrene ring, an anthraanthrene ring, and a tetraline ring); an aromatic heterocyclic group (for example, groups derived from a furan ring, a dibenzofuran ring, a thiophene ring, a dibenzothiophene ring, an oxazole ring, a pyrrole ring, a pyridine ring, a pyridazine ring, a pyrimidine ring, a pyrazine ring, a triazine ring, a benzimidazole ring, an oxadiazole ring, a triazole ring, an imidazole ring, a pyrazole ring, a thiazole ring, an indole ring, an indazole ring, a benzimidazole ring, a benzothiazole ring, a benzoxazole ring, a quinoxaline ring, a quinazoline ring, a cinnoline ring, a quinoline ring, an isoquinoline ring, a phthalazine ring, a naphthylidine ring, a carbazole ring, carboline ring, a diazacarbazole ring (indicating a ring structure in which one of the carbon atoms constituting the carboline ring is further replaced with a nitrogen atom); a non-aromatic hydrocarbon ring group (for example, a cyclopentyl group and a cyclohexyl group); and a non-aromatic heterocyclic group (for example, a pyrrolidyl group, an imidazolidyl group, a morpholyl group, and an oxazolidyl group); an alkoxy group (for example, a methoxy group, an ethoxy group, a propyloxy group, a pentyloxy group, an hexyloxy group, an octyloxy group, and a dodecyloxy group); a cycloalkoxy group (for example, a cyclopentyloxy group and a cyclohexyloxy group); an aryloxy group (for example, a phenoxy group and a naphthyloxy group); an alkylthio group (for example, a methylthio group, an ethylthio group, a propylthio group, a pentylthio group, hexylthio group, an octylthio group, and a dodecylthio group); a cycloalkylthio group (for example, a cyclopentylthio group and a cyclohexylthio group); an arylthio group (for example, a phenylthio group and a naphthylthio group); an alkoxycarbonyl group (for example, a methyloxycarbonyl group, an ethyloxycarbonyl group, a butyloxycarbonyl group, an octyloxycarbonyl group, and a dodecyloxycarbonyl group); an aryloxycarbonyl group (for example, a phenyloxycarbonyl group and a naphthyloxycarbonyl group); a sulfamoyl group (for example, an aminosulfonyl group, a methylaminosulfonyl group, a dimethylaminosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, and a 2-pyridylaminosulfonyl group); an acyl group (for example, an acetyl group, an ethyl carbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, and a pyridylcarbonyl group); an acyloxy group (for example, an acetyloxy group, an ethylcarbonyloxy group, a butylcarbonyloxy group, an octylcarbonyloxy group, a dodecylcarbonyloxy group, and a phenylcarbonyloxy group); an amido group (for example, a methylcarbonylamino group, an ethylcarbonylamino group, a dimethylcarbonylamino group, a propylcarbonylamino group, a pentylcarbonylamino group, a cyclohexylcarbonylamino group, a 2-ethylhexylcarbonylamino group, an octylcarbonylamino group, a dodecylcarbonylamino group, a phenylcarbonylamino group, and a naphthylcarbonylamino group); a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethymexylaminocarbonyl group, a dodecylaminocarbonyl group, a phenylaminocarbonyl group, a naphthylaminocarbonyl group, and a 2-pyridylaminocarbonyl group); a ureido group (for example, a methylureido group, an ethylureido group, a pentylureido group, a cyclohexylureido group, an octylureido group, a dodecylureido group, a phenylureido group, a naphthylureido group, and a 2-pyridylaminoureido group); a sulfinyl group (for example, a methylsulfinyl group, an ethylsufinyl group, a butylsulfinyl group, a cyclohexylsulfinyl group, a 2-ethylhexylsulfinyl group, a dodecylsulfinyl group, a phenylsulfinyl group, a naphthylsulfinyl group, and a 2-pyridylsulfinyl group); an alkylsulfonyl group (for example, a methylsulfonyl group, an ethylsulfonyl group, a butylsulfinyl group, a cyclohexylsulfonyl group, a 2-ethylhexylsulfonyl group, and a dodecylsulfonyl group); an arylsulfonyl group or a heteroarylsulfonyl group (for example, a phenylsulfonyl group, a naphthylsulfonyl group, and a 2-pyridylsulfonyl group); an amino group (for example, an amino group, an ethylamino group, a dimethylamino group, a butylamino group, a cyclopentylamino group, a dodecylamino group, an anilino group, a naphthylamino group, and a 2-pyridylamino group); a halogen atom (for example, a fluorine atom, a chlorine atom and a bromine atom); a fluorinated hydrocarbon group (for example, a fluoromethyl group, trifluoromethyl group, a pentafluoroethyl group and a pentafluorophenyl group); a cyano group; a nitro group; a hydroxy group; a mercapto group; a silyl group (for example, a trimethylsilyl group, a triisopropylsilyl group, a triphenylsilyl group, and a phenyldiethylsilyl group) and a deuterium atom.

These substituents may be further substituted with the above-mentioned substituents. Further, two or more of these substituents may be combined to form a ring.

Among the structures represented by Formula (3), compounds in which X₁₀₁ is NR₁₀₁, an oxygen atom or a sulfur atom are preferable. More preferably, the condensed ring formed with X₁₀₁ and y₁ to y₈ is a carbazole ring, an azacarbazole ring, a dibenzofuran ring or an azadibenzofuran ring.

In Formula (3), n represents an integer of 1 to 4, preferably 1 to 2.

Further, R of Formula (2) represents a substituent as in R₁₀₁ to R₁₀₆, but a substituent that improves the solubility is preferable.

Examples of such substituents include a straight or branched alkyl group (for example, a methyl group, an ethyl group, a propyl group, an isopropyl group, a tert-butyl group, a pentyl group, a hexyl group, an octyl group, a dodecyl group, a tridecyl group, a tetradecyl group, and a pentadecyl group); an aromatic hydrocarbon ring group (also referred to as an aromatic carbocyclic group or an aryl group, for example, groups derived from a phenyl ring, a biphenyl ring, a naphthalene ring, an azulene ring, an anthracene ring, a phenanthrene ring, a pyrene ring, a chrysene ring, a naphthacene ring, a triphenylene ring, an o-terphenyl ring, a m-terphenyl ring, a p-terphenyl ring, an acenaphthene ring, a coronene ring, an inden ring, a fluorene ring, a fluoranthene ring, a naphthalene ring, a pentacene ring, a perylene ring, a pentaphene ring, a picene ring, a pyrene ring, a pyranthrene ring, an anthraanthrene ring, and a tetraline ring); an aromatic heterocyclic group (for example, groups derived from a furan ring, a dibenzofuran ring, a thiophene ring, a dibenzothiophene ring, an oxazole ring, a pyrrole ring, a pyridine ring, a pyridazine ring, a pyrimidine ring, a pyrazine ring, a triazine ring, a benzimidazole ring, an oxadiazole ring, a triazole ring, an imidazole ring, a pyrazole ring, a thiazole ring, an indole ring, an indazole ring, a benzimidazole ring, a benzothiazole ring, a benzoxazole ring, a quinoxaline ring, a quinazoline ring, a cinnoline ring, a quinoline ring, an isoquinoline ring, a phthalazine ring, a naphthylidene ring, a carbazole ring, carboline ring, a diazacarbazole ring (indicating a ring structure in which one of the carbon atoms constituting the carboline ring is further replaced with a nitrogen atom); a non-aromatic hydrocarbon ring group (for example, a cyclopentyl group and a cyclohexyl group); and a non-aromatic heterocyclic group (for example, a pyrrolidyl group, an imidazolidyl group, a morpholyl group, and an oxazolidyl group).

In Formula (I), it is preferable that any substituent of X₁ to X₅ has an electron-transporting structure and a hole-transporting structure from the viewpoint of the suitability for application to a charge transfer/light-emitting thin film.

In the present invention, the electron-transporting structure is a structure having a function of transporting electrons, and it may be, for example, a structure having any of electron injection property or transport property, and hole barrier property.

Specific preferable structures are aromatic heterocyclic groups (for example, a furan ring, a dibenzofuran ring, a thiophene ring, a dibenzothiophene ring, an oxazole ring, a pyridine ring, a pyridazine ring, a pyrimidine ring, a pyrazine ring, a triazine ring, a benzimidazole ring, an oxadiazole ring, a triazole ring, an imidazole ring, a pyrazole ring, a thiazole ring, an indole ring, an indazole ring, a benzimidazole ring, a benzothiazole ring, a benzoxazole ring, a quinoxaline ring, a quinazoline ring, a cinnoline ring, a quinoline ring, an isoquinoline ring, a phthalazine ring, a naphthylidine ring, carboline ring, and a diazacarbazole ring).

The hole-transporting structure is a structure having a function of transporting holes, and it may be, for example, a structure having any of hole injecting property or transporting property, and electron barrier properly.

As a specific structure, an arylamine structure and an alkylamine structure are preferable.

Exemplary compounds of the π-conjugated compound having the structure of Formula (I) are shown below, but the present invention is not limited thereto.

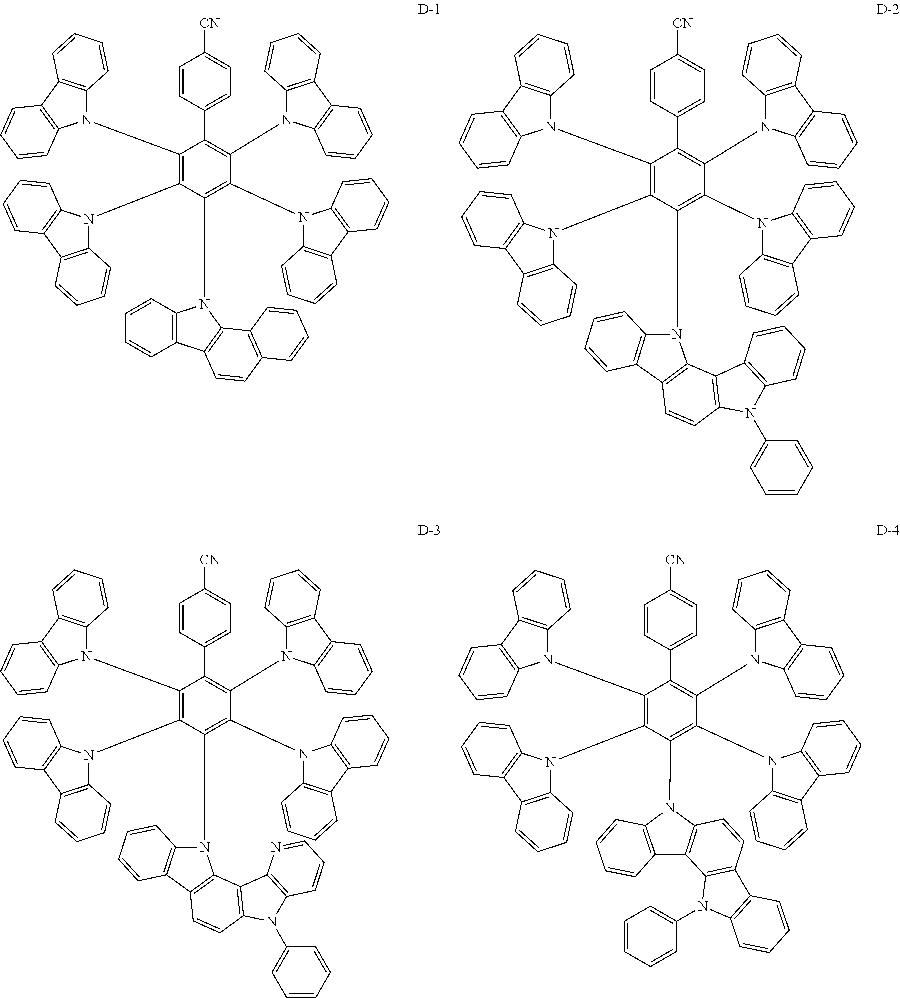

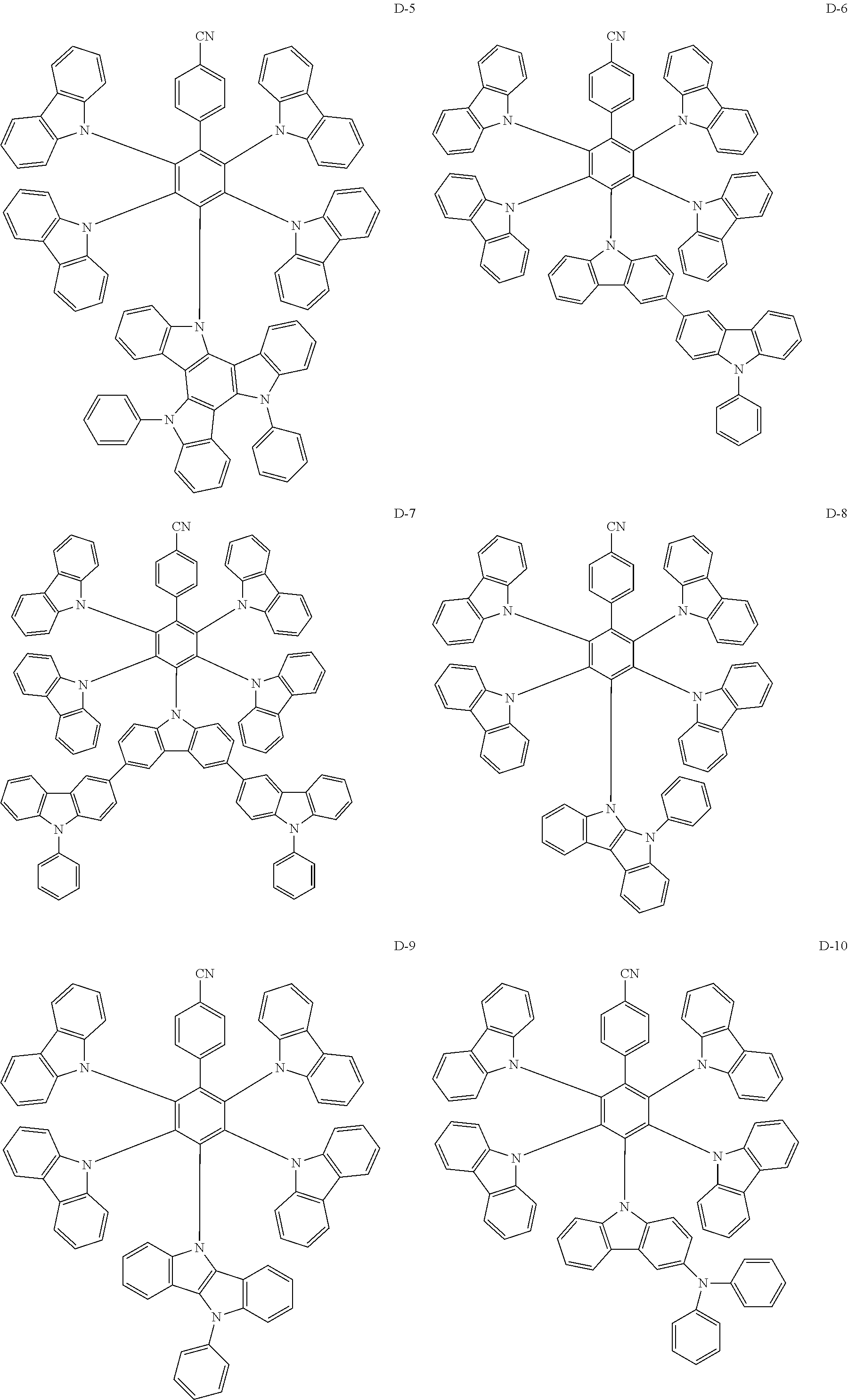

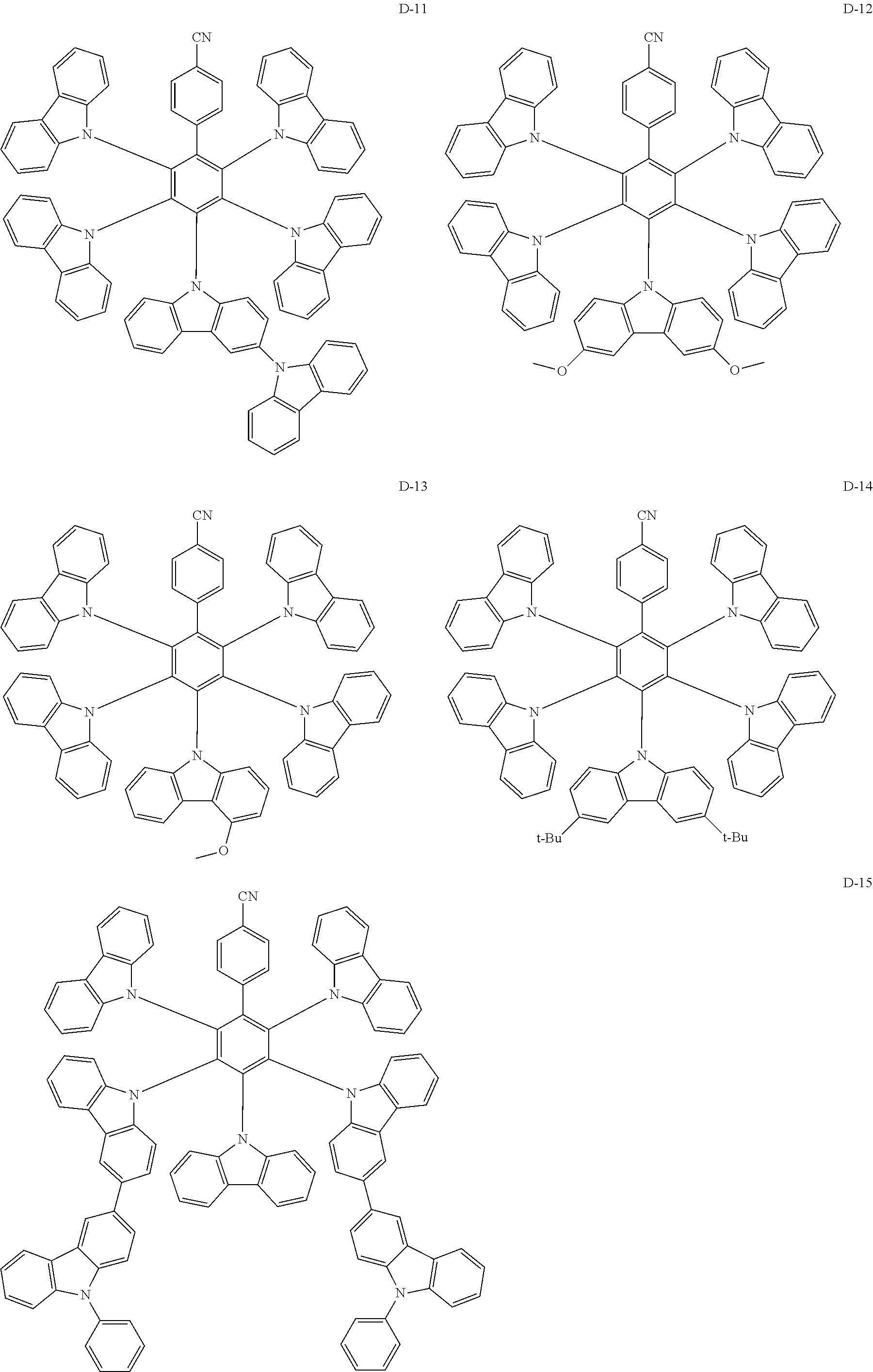

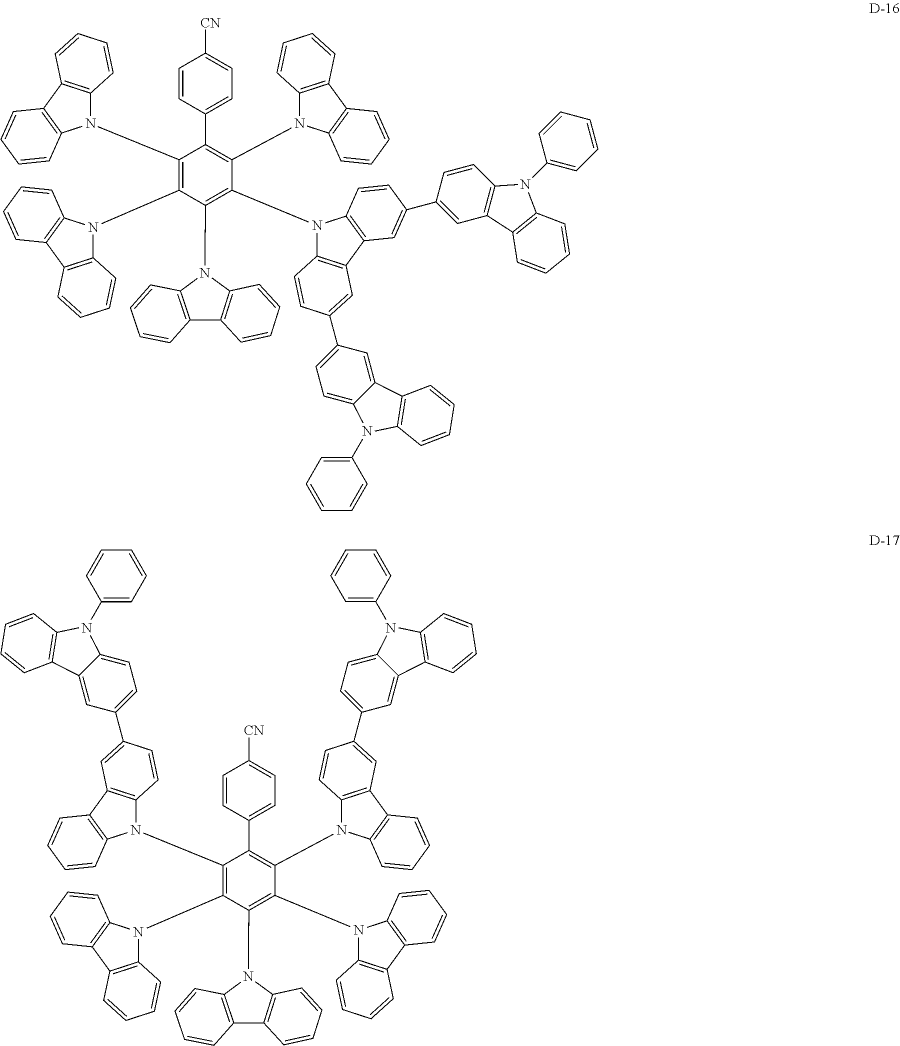

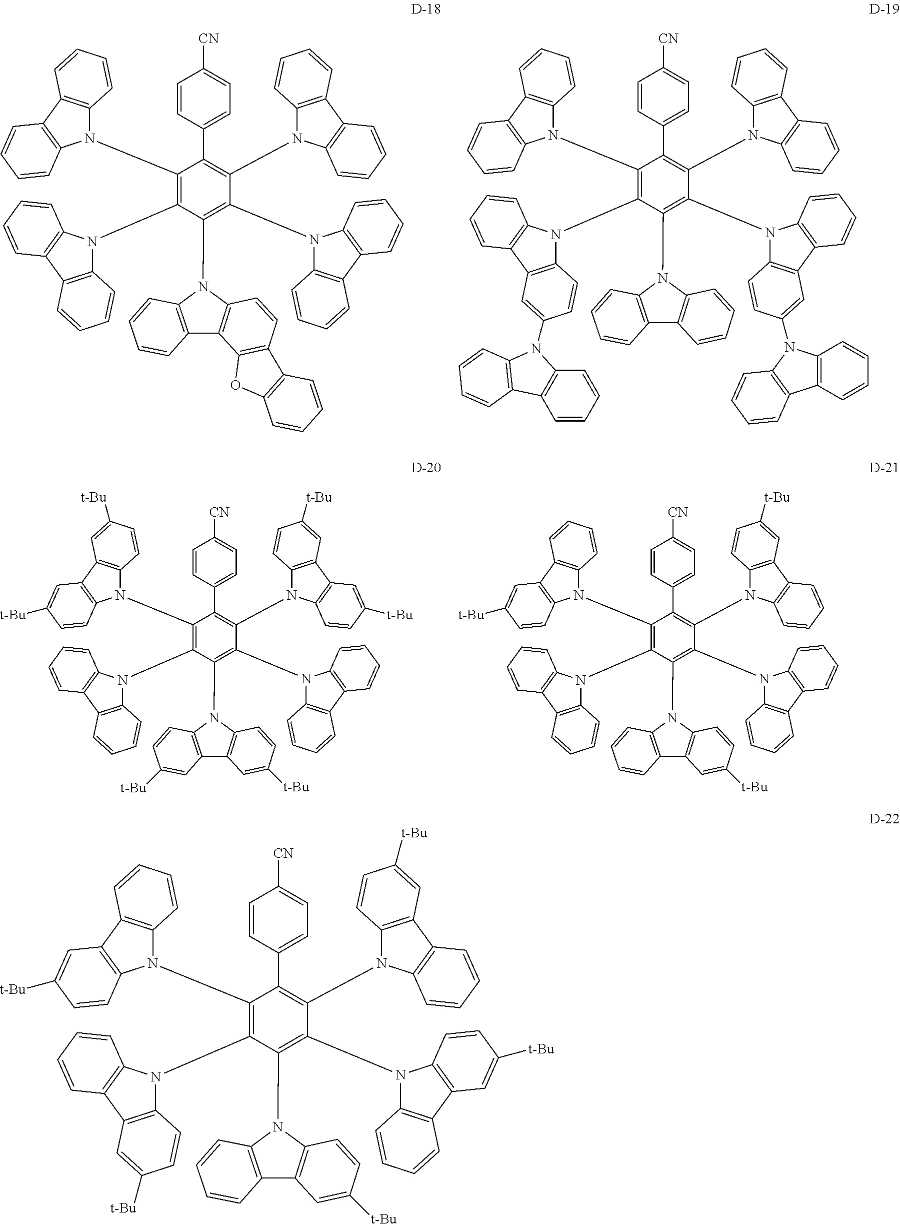

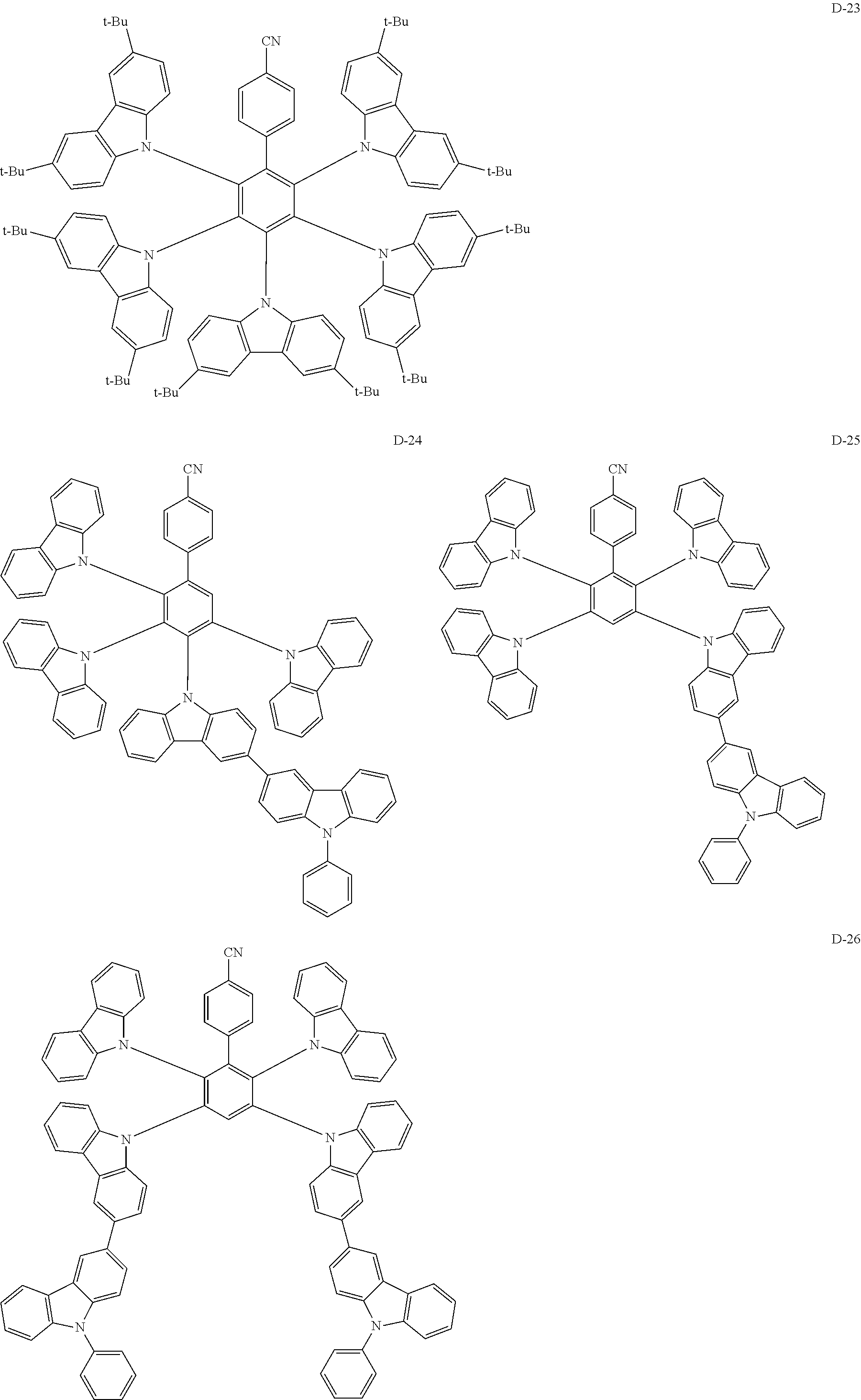

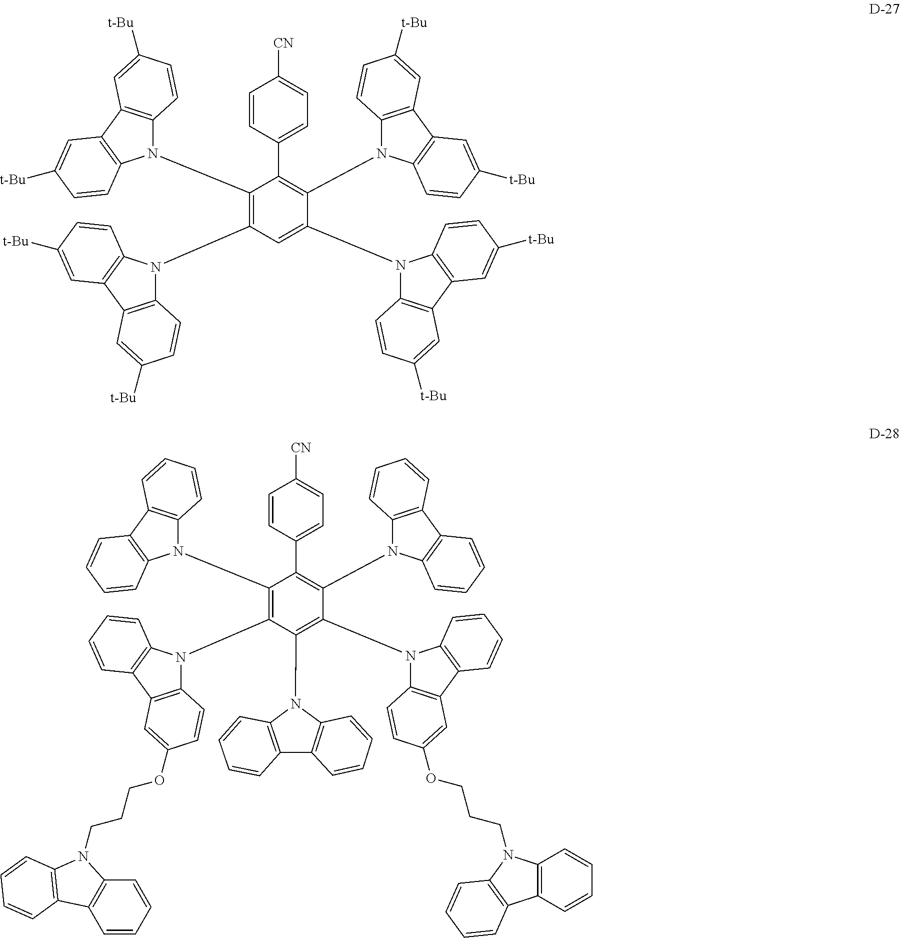 [SMILES: CC(C)(C)c1ccc2c(c1)c1cc(C(C)(C)C)ccc1n2-c1cc(-n2c3ccc(C(C)(C)C)cc3c3cc(C(C)(C)C)ccc32)c(-n2c3ccc(C(C)(C)C)cc3c3cc(C(C)(C)C)ccc32)c(-c2ccc(C#N)cc2)c1-n1c2ccc(C(C)(C)C)cc2c2cc(C(C)(C)C)ccc21 ; N#Cc1ccc(-c2c(-n3c4ccccc4c4ccccc43)c(-n3c4ccccc4c4cc(OCCCn5c6ccccc6c6ccccc65)ccc43)c(-n3c4ccccc4c4ccccc43)c(-n3c4ccccc4c4cc(OCCCn5c6ccccc6c6ccccc65)ccc43)c2-n2c3ccccc3c3ccccc32)cc1]

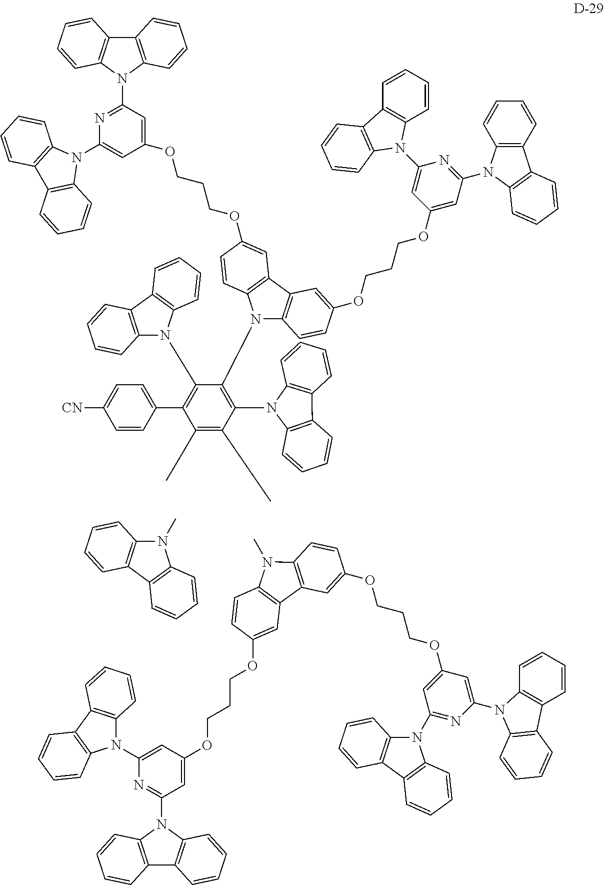

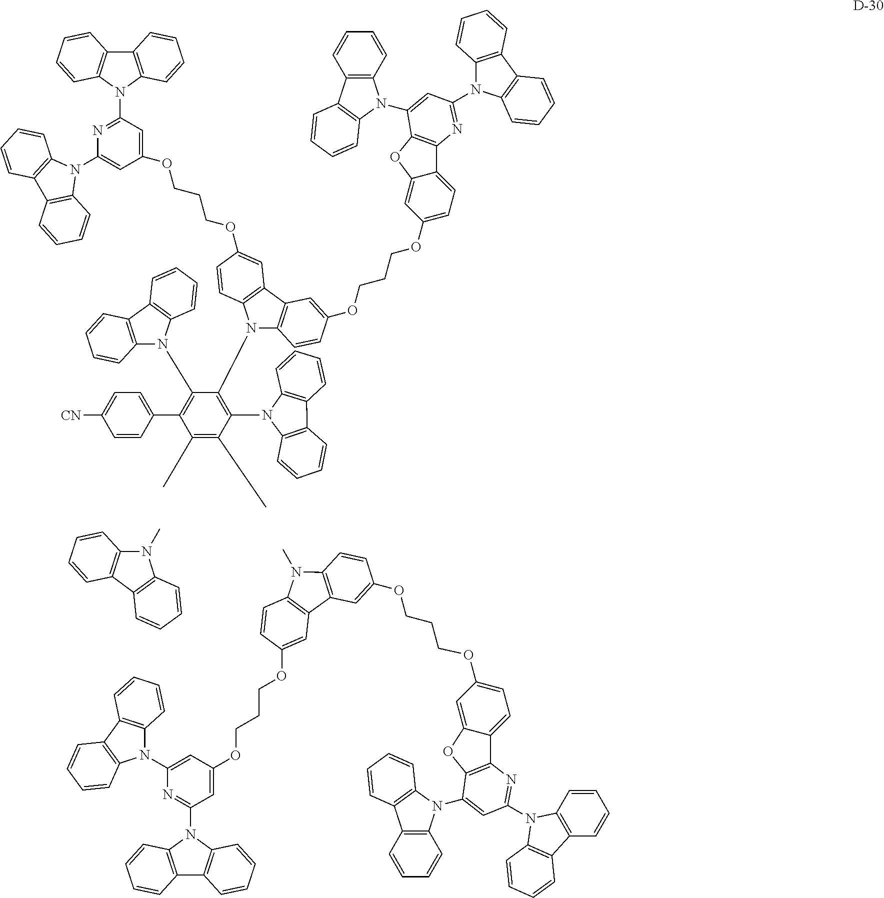

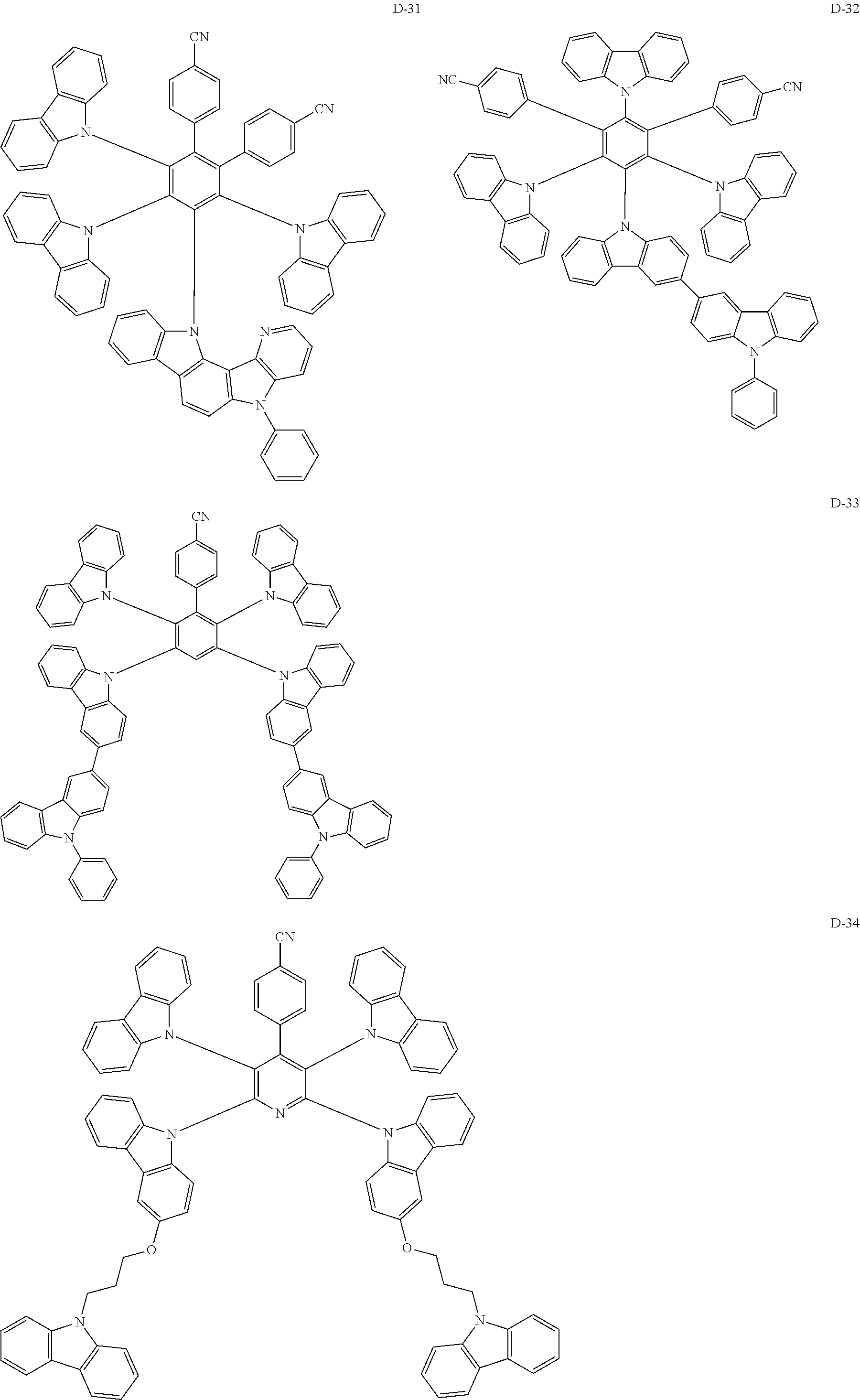

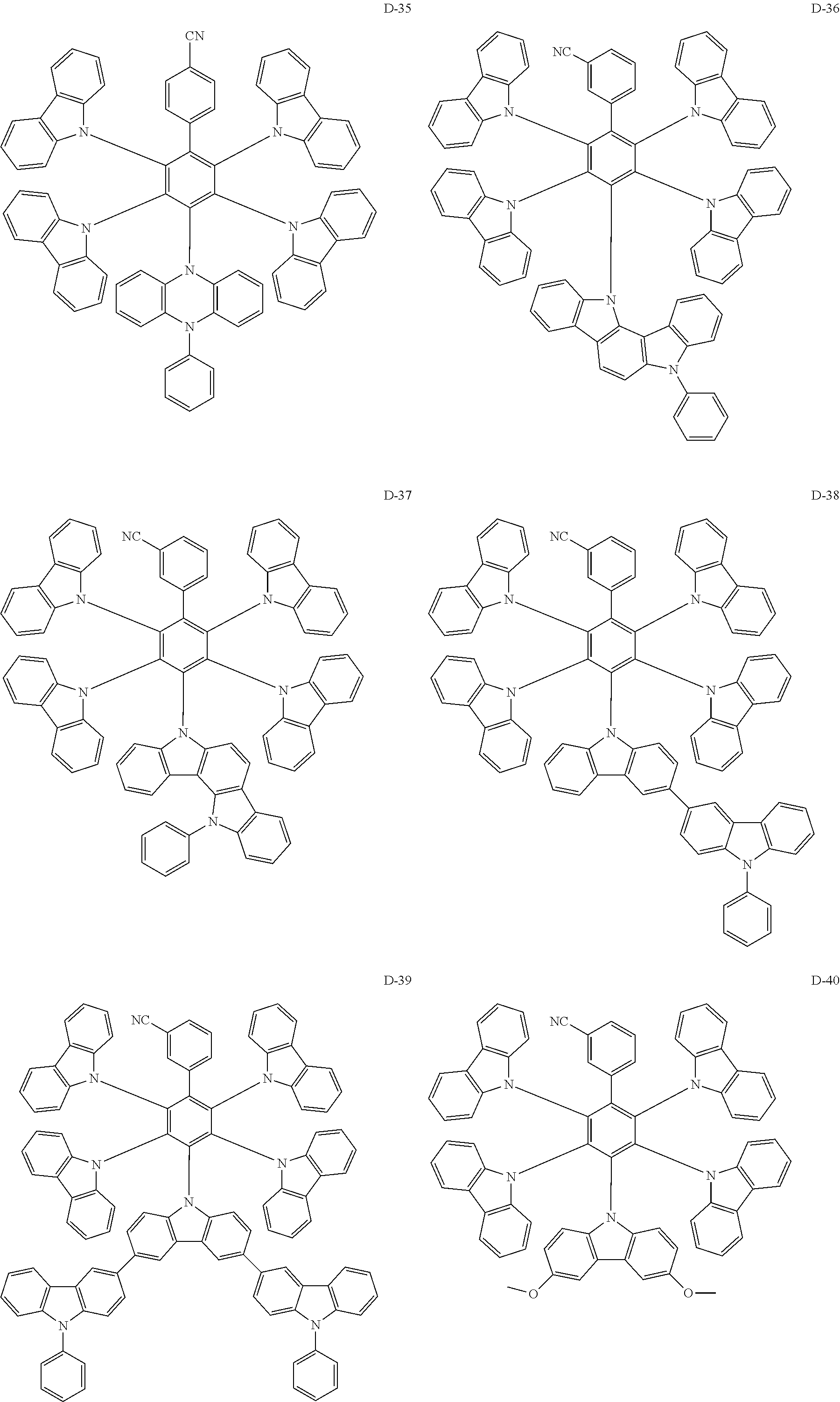

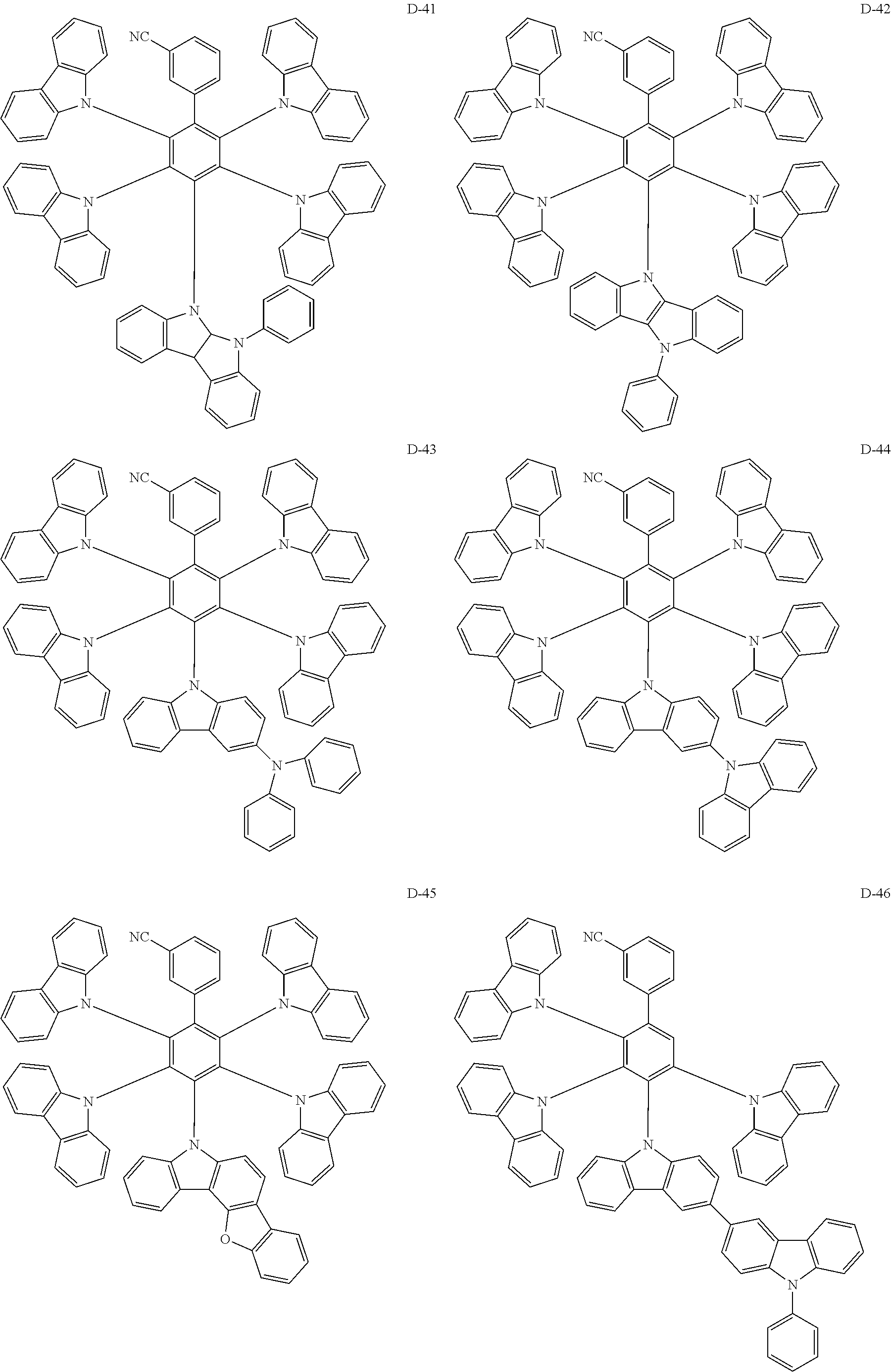

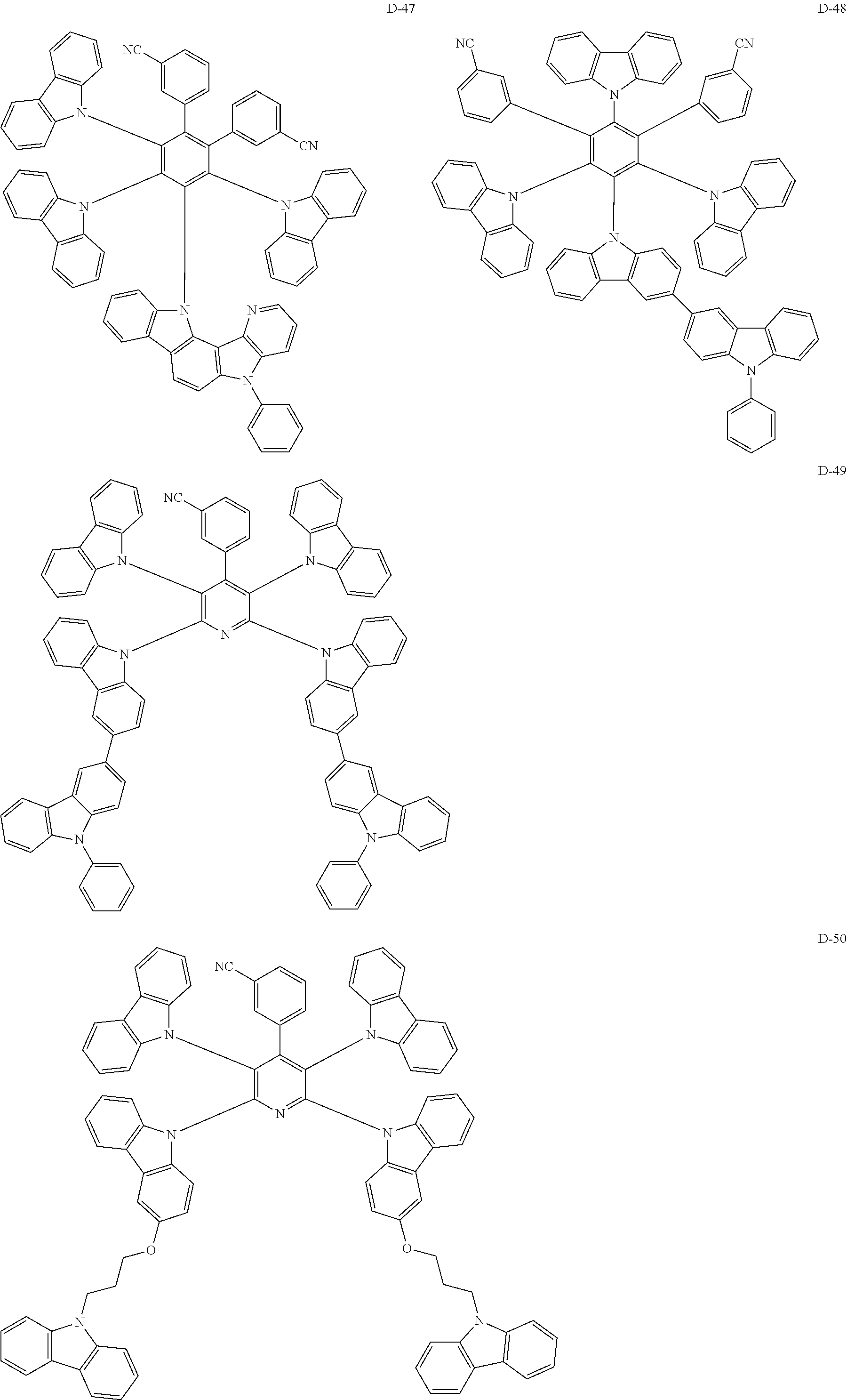

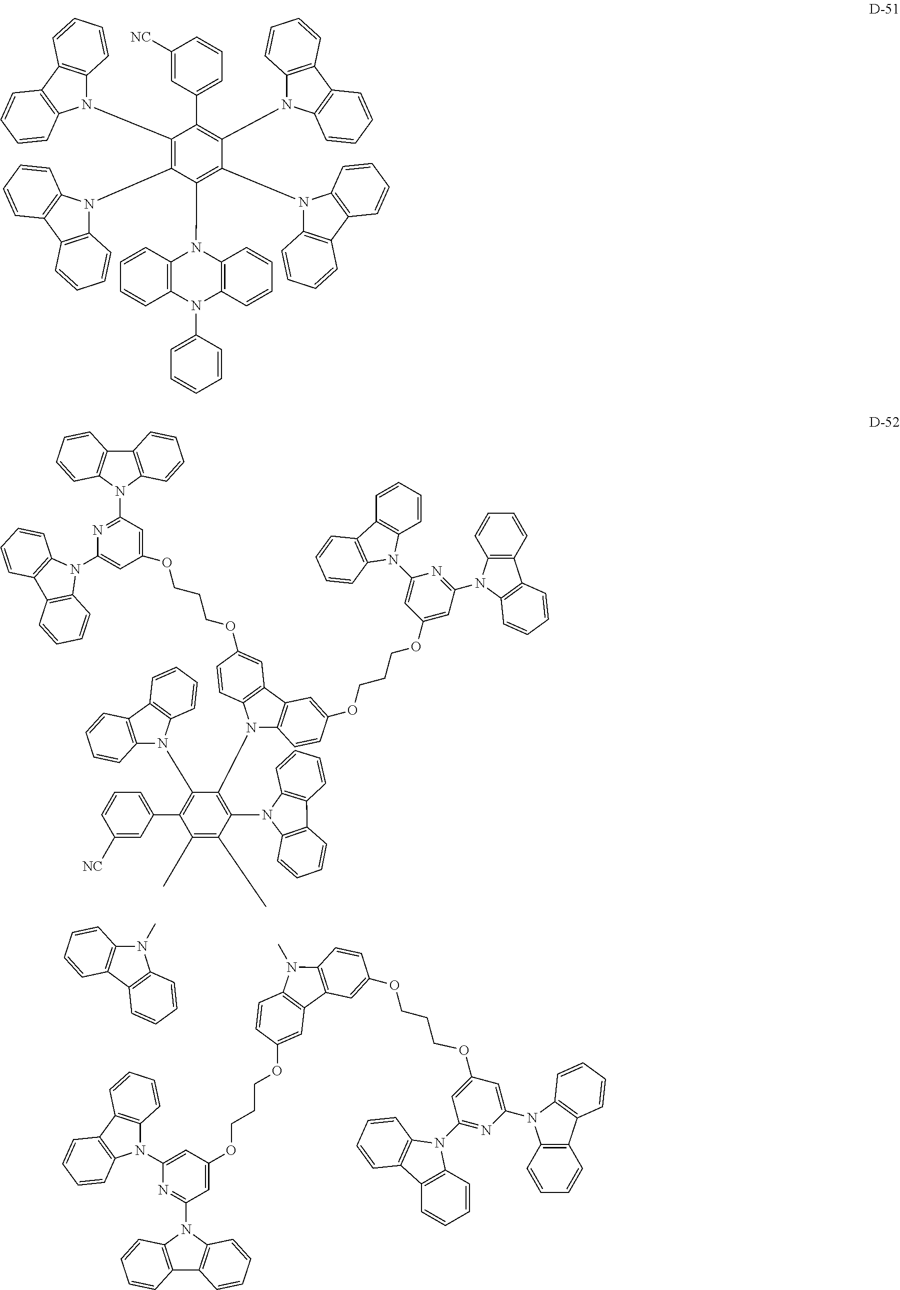

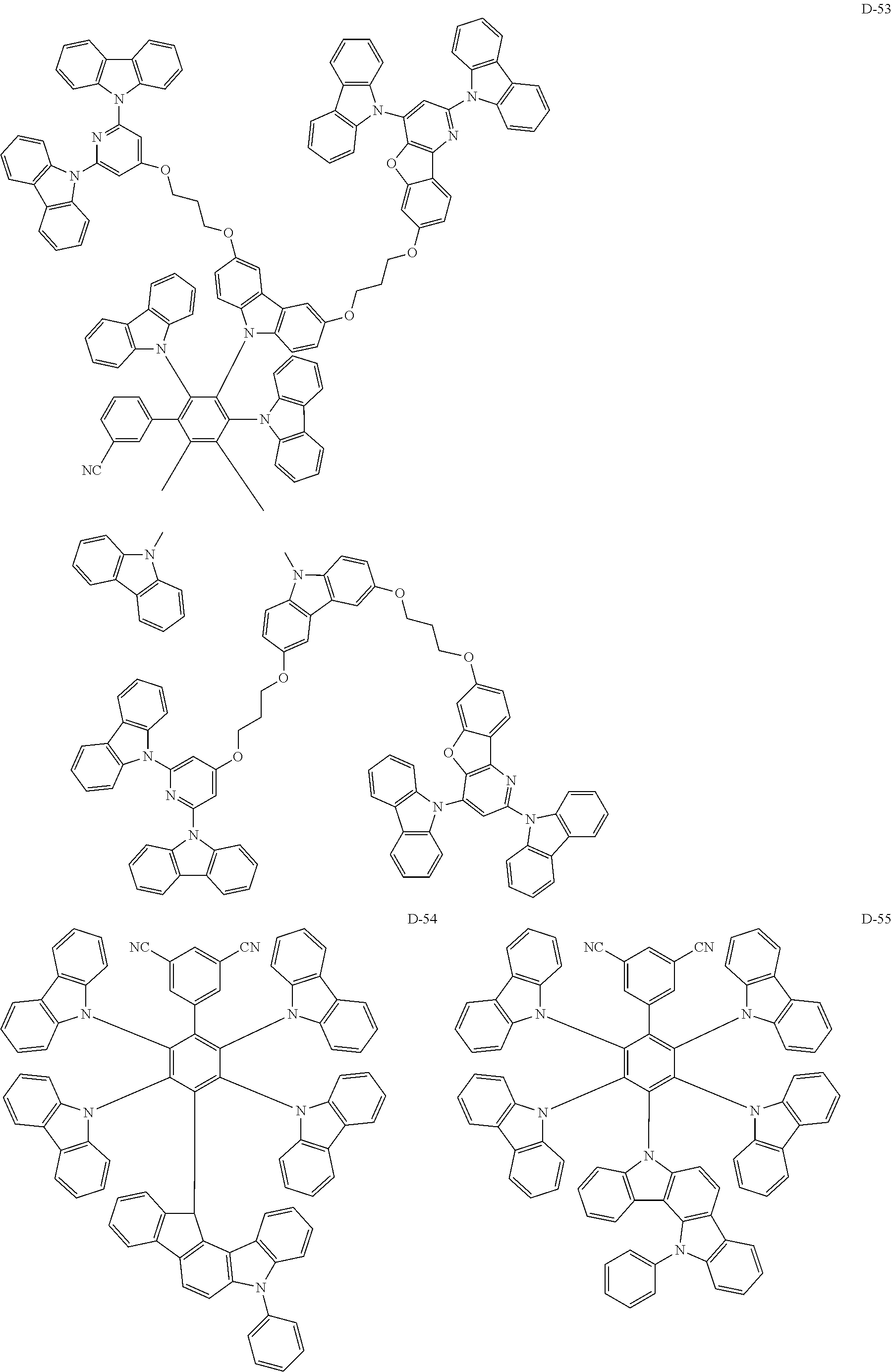

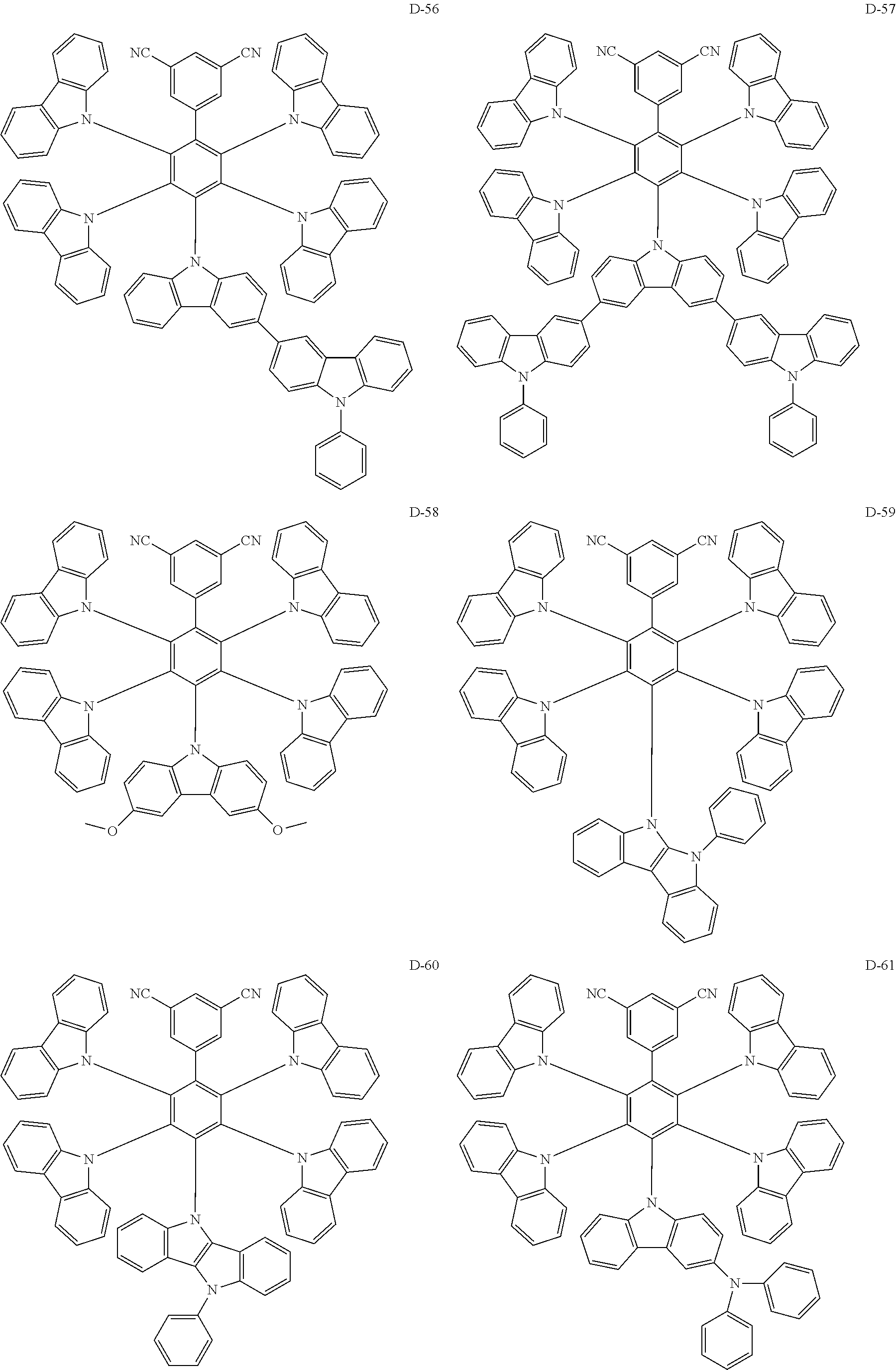

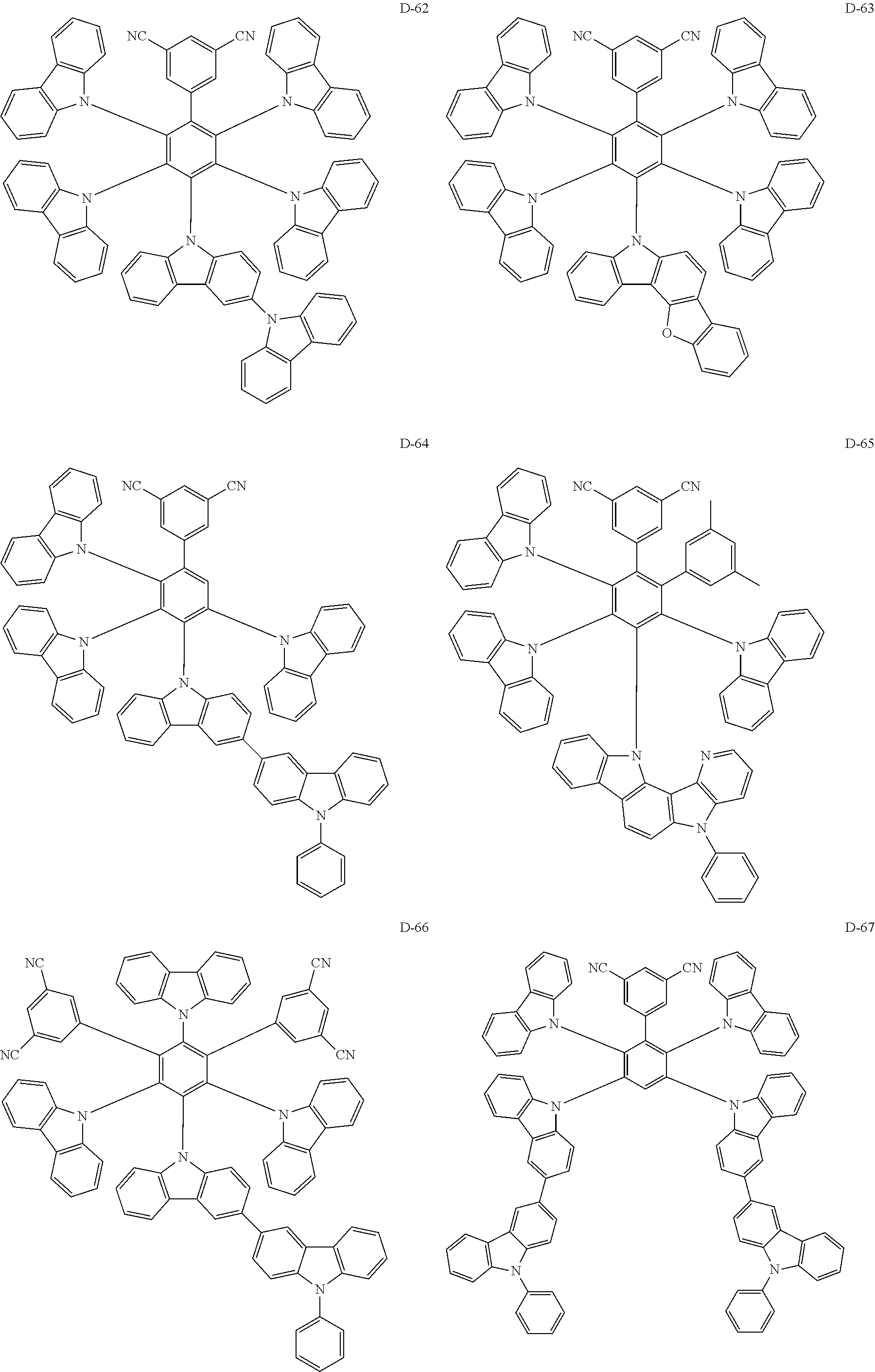

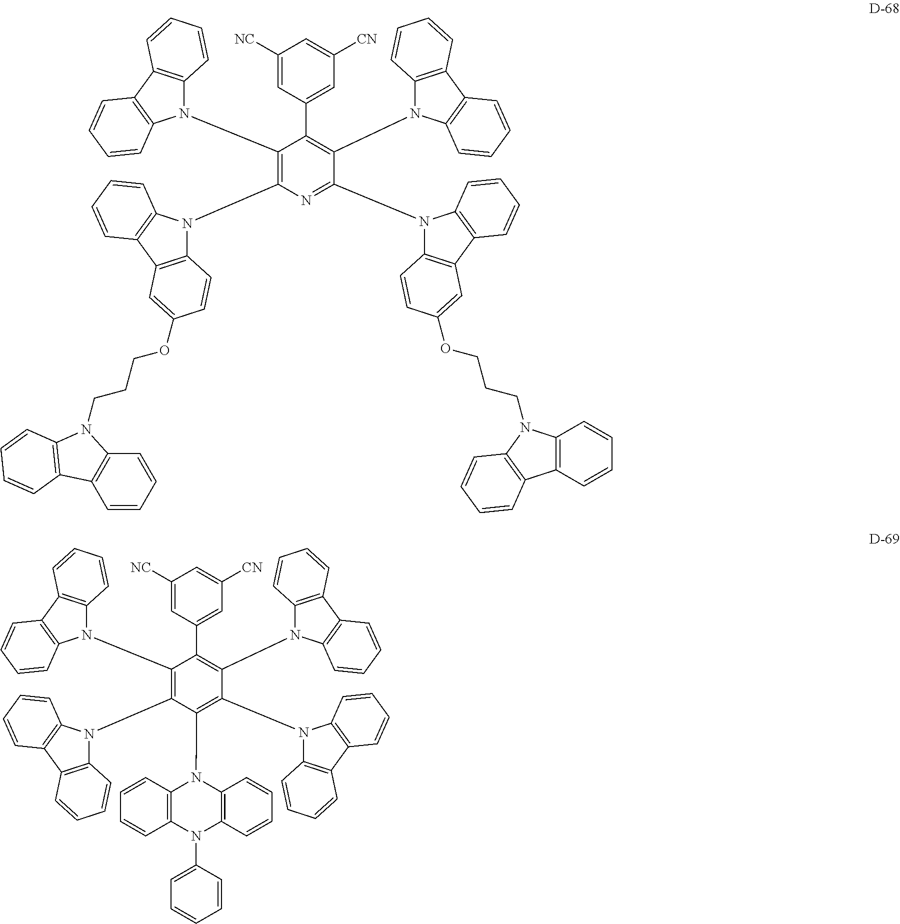 [SMILES: N#Cc1cc(C#N)cc(-c2c(-n3c4ccccc4c4ccccc43)c(-n3c4ccccc4c4cc(OCCCn5c6ccccc6c6ccccc65)ccc43)nc(-n3c4ccccc4c4cc(OCCCn5c6ccccc6c6ccccc65)ccc43)c2-n2c3ccccc3c3ccccc32)c1 ; N#Cc1cc(C#N)cc(-c2c(-n3c4ccccc4c4ccccc43)c(-n3c4ccccc4c4ccccc43)c(N3c4ccccc4N(c4ccccc4)c4ccccc43)c(-n3c4ccccc4c4ccccc43)c2-n2c3ccccc3c3ccccc32)c1]

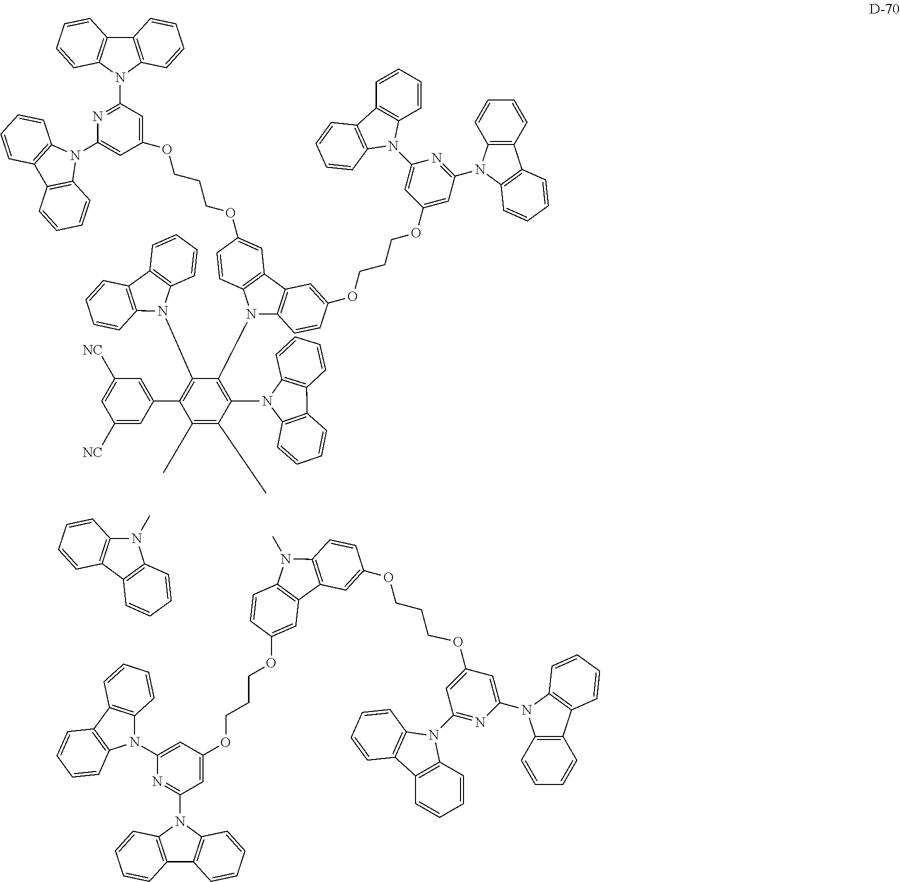 [SMILES: Cc1c(C)c(-n2c3ccccc3c3ccccc32)c(-n2c3ccc(OCCCOc4cc(-n5c6ccccc6c6ccccc65)nc(-n5c6ccccc6c6ccccc65)c4)cc3c3cc(OCCCOc4cc(-n5c6ccccc6c6ccccc65)nc(-n5c6ccccc6c6ccccc65)c4)ccc32)c(-n2c3ccccc3c3ccccc32)c1-c1cc(C#N)cc(C#N)c1 ; Cn1c2ccc(OCCCOc3cc(-n4c5ccccc5c5ccccc54)nc(-n4c5ccccc5c5ccccc54)c3)cc2c2cc(OCCCOc3cc(-n4c5ccccc5c5ccccc54)nc(-n4c5ccccc5c5ccccc54)c3)ccc21 ; Cn1c2ccccc2c2ccccc21]

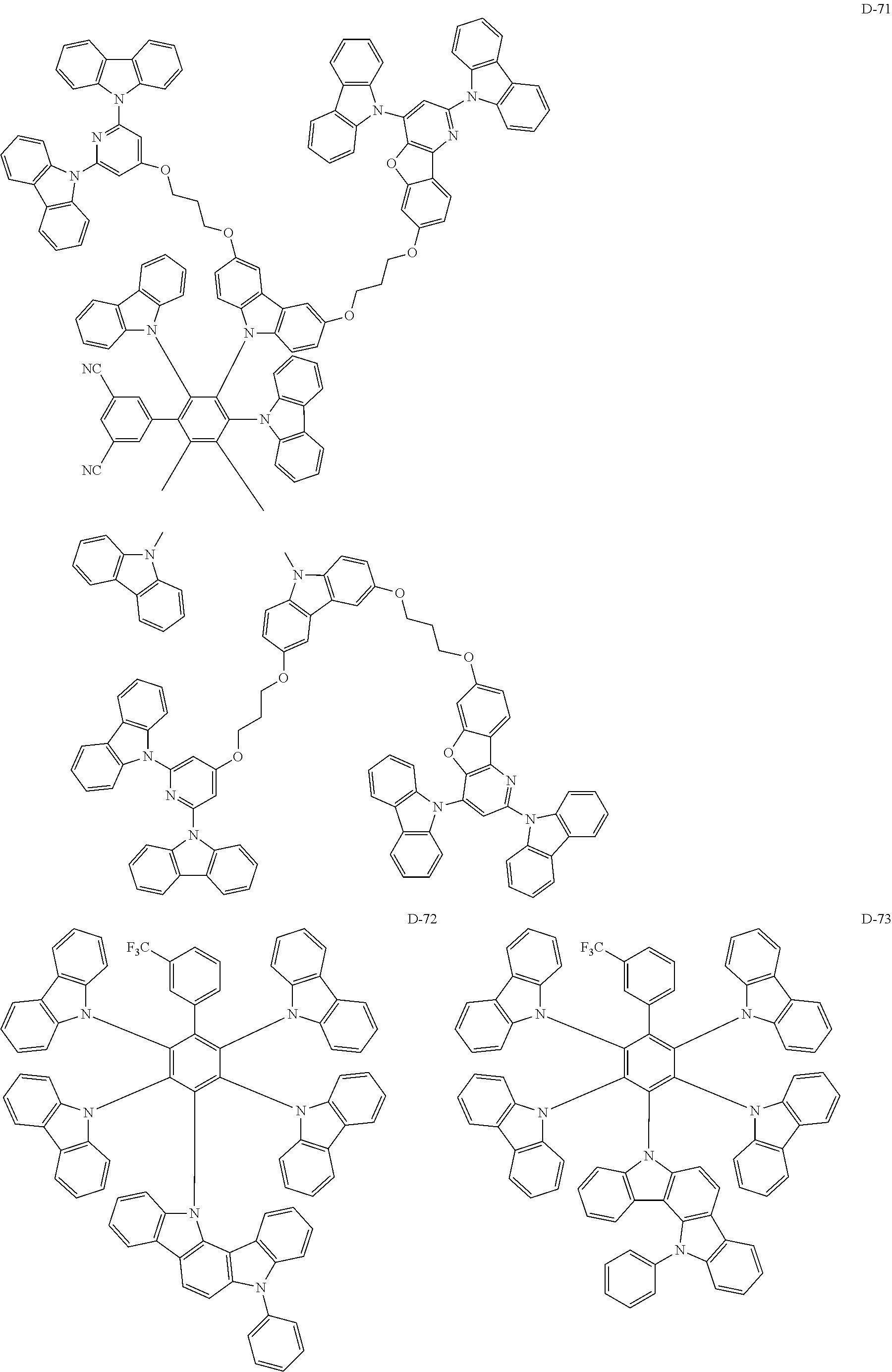

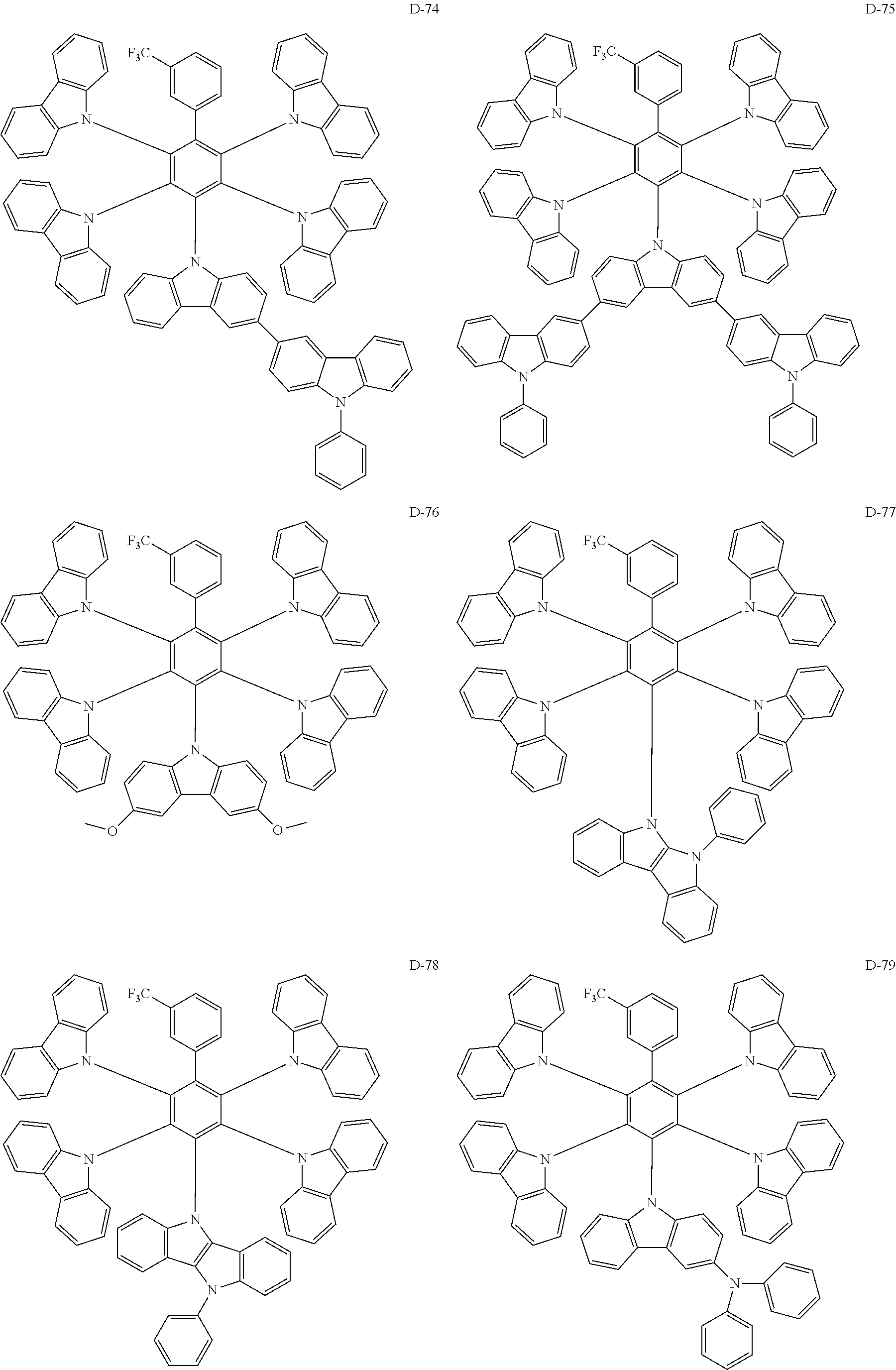

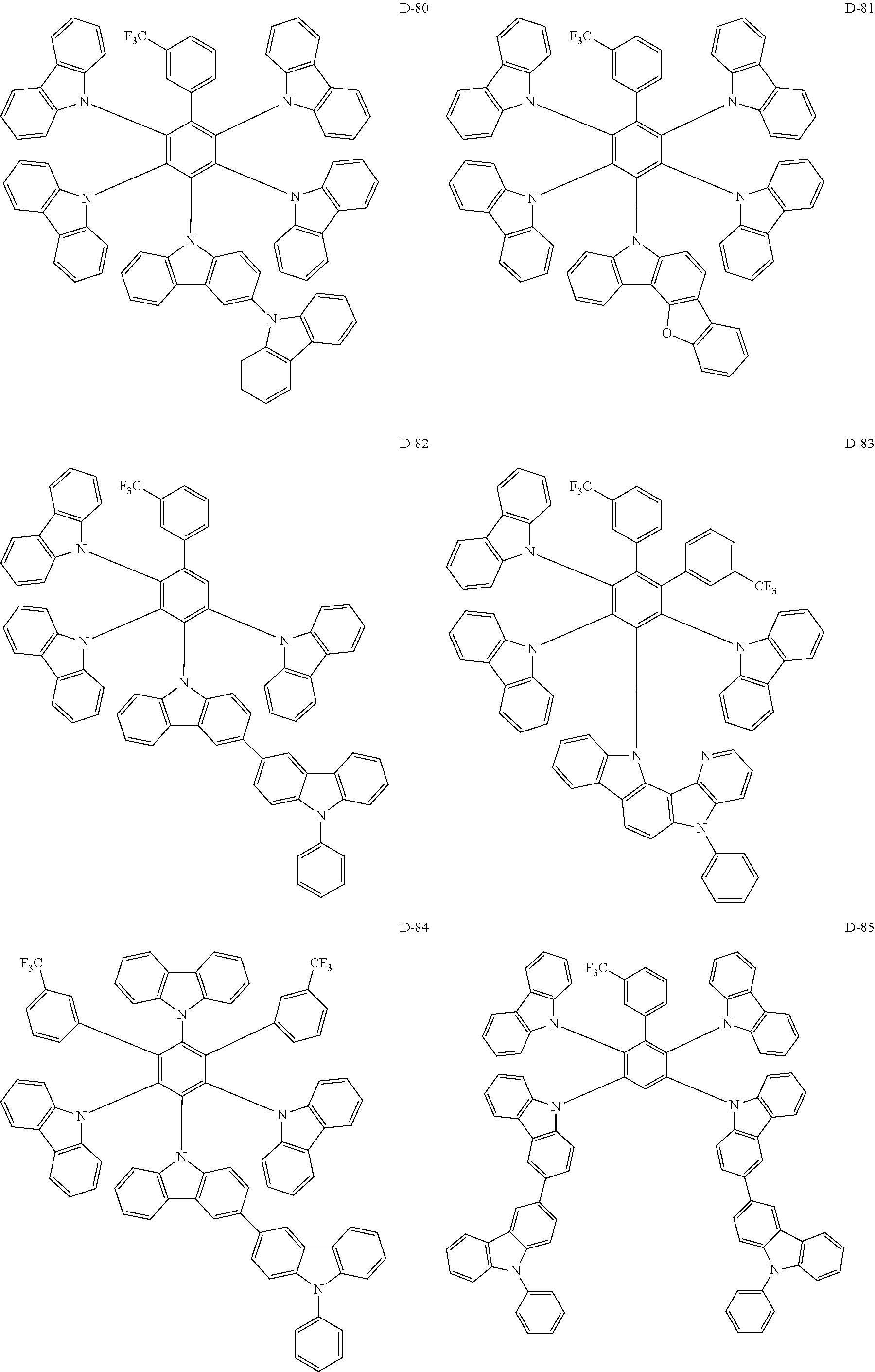

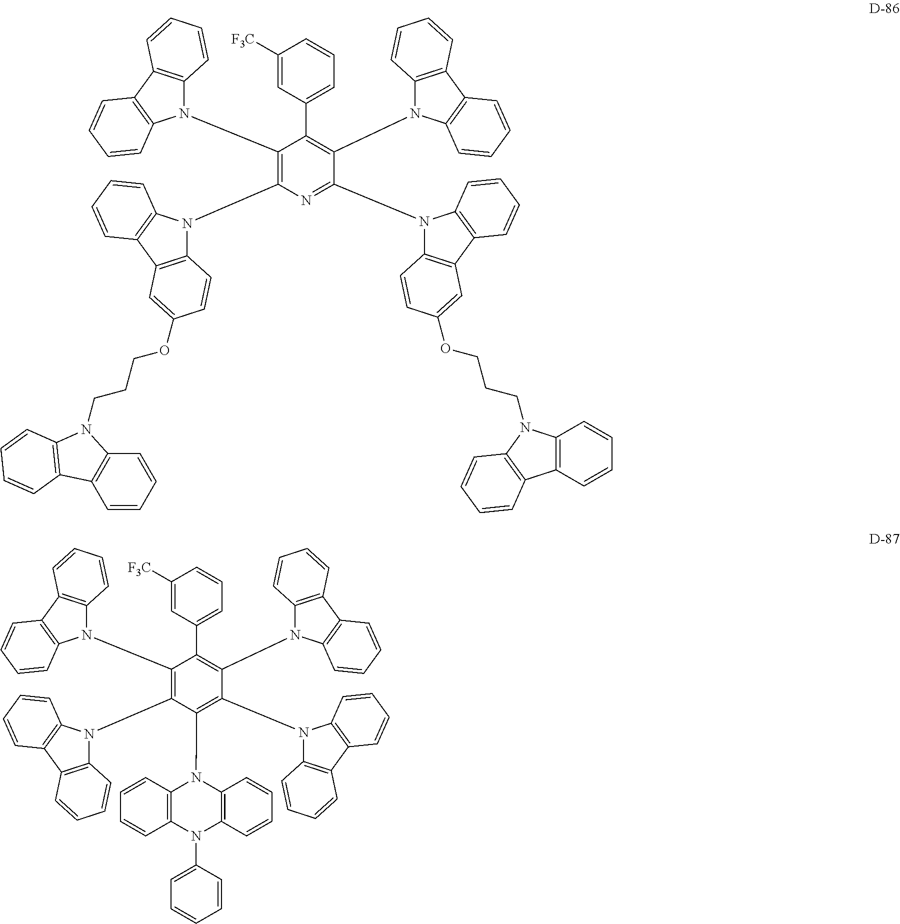 [SMILES: FC(F)(F)c1cccc(-c2c(-n3c4ccccc4c4ccccc43)c(-n3c4ccccc4c4cc(OCCCn5c6ccccc6c6ccccc65)ccc43)nc(-n3c4ccccc4c4cc(OCCCn5c6ccccc6c6ccccc65)ccc43)c2-n2c3ccccc3c3ccccc32)c1 ; FC(F)(F)c1cccc(-c2c(-n3c4ccccc4c4ccccc43)c(-n3c4ccccc4c4ccccc43)c(N3c4ccccc4N(c4ccccc4)c4ccccc43)c(-n3c4ccccc4c4ccccc43)c2-n2c3ccccc3c3ccccc32)c1]

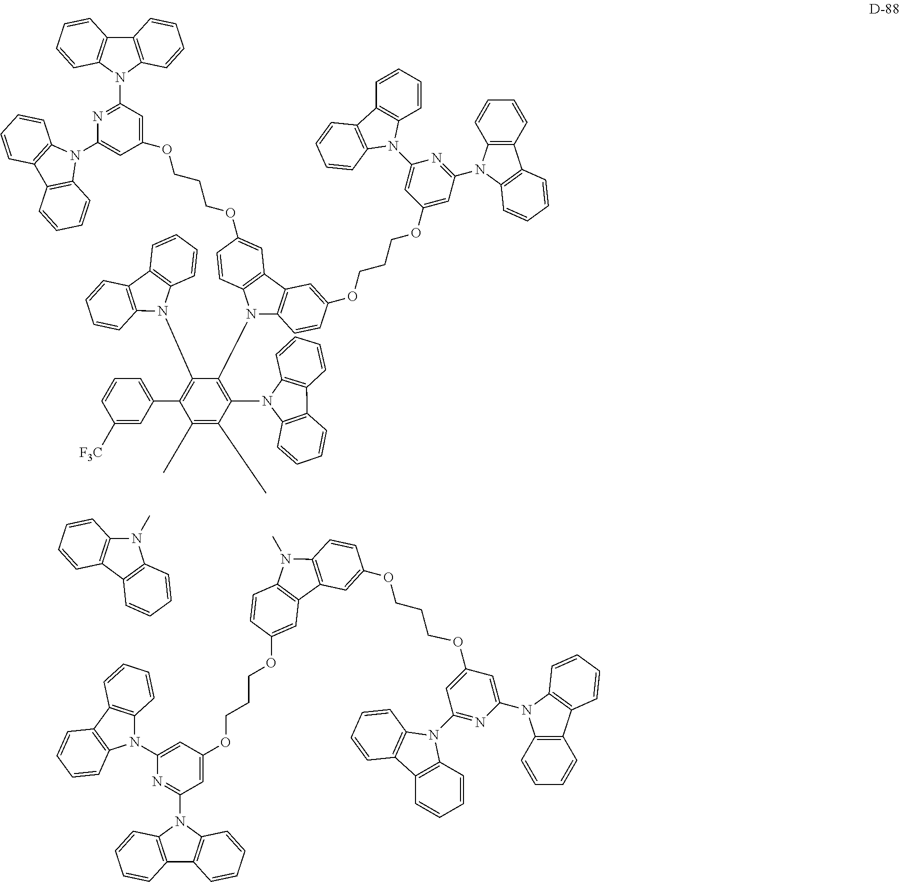 [SMILES: Cc1c(C)c(-n2c3ccccc3c3ccccc32)c(-n2c3ccc(OCCCOc4cc(-n5c6ccccc6c6ccccc65)nc(-n5c6ccccc6c6ccccc65)c4)cc3c3cc(OCCCOc4cc(-n5c6ccccc6c6ccccc65)nc(-n5c6ccccc6c6ccccc65)c4)ccc32)c(-n2c3ccccc3c3ccccc32)c1-c1cccc(C(F)(F)F)c1 ; Cn1c2ccc(OCCCOc3cc(-n4c5ccccc5c5ccccc54)nc(-n4c5ccccc5c5ccccc54)c3)cc2c2cc(OCCCOc3cc(-n4c5ccccc5c5ccccc54)nc(-n4c5ccccc5c5ccccc54)c3)ccc21 ; Cn1c2ccccc2c2ccccc21]

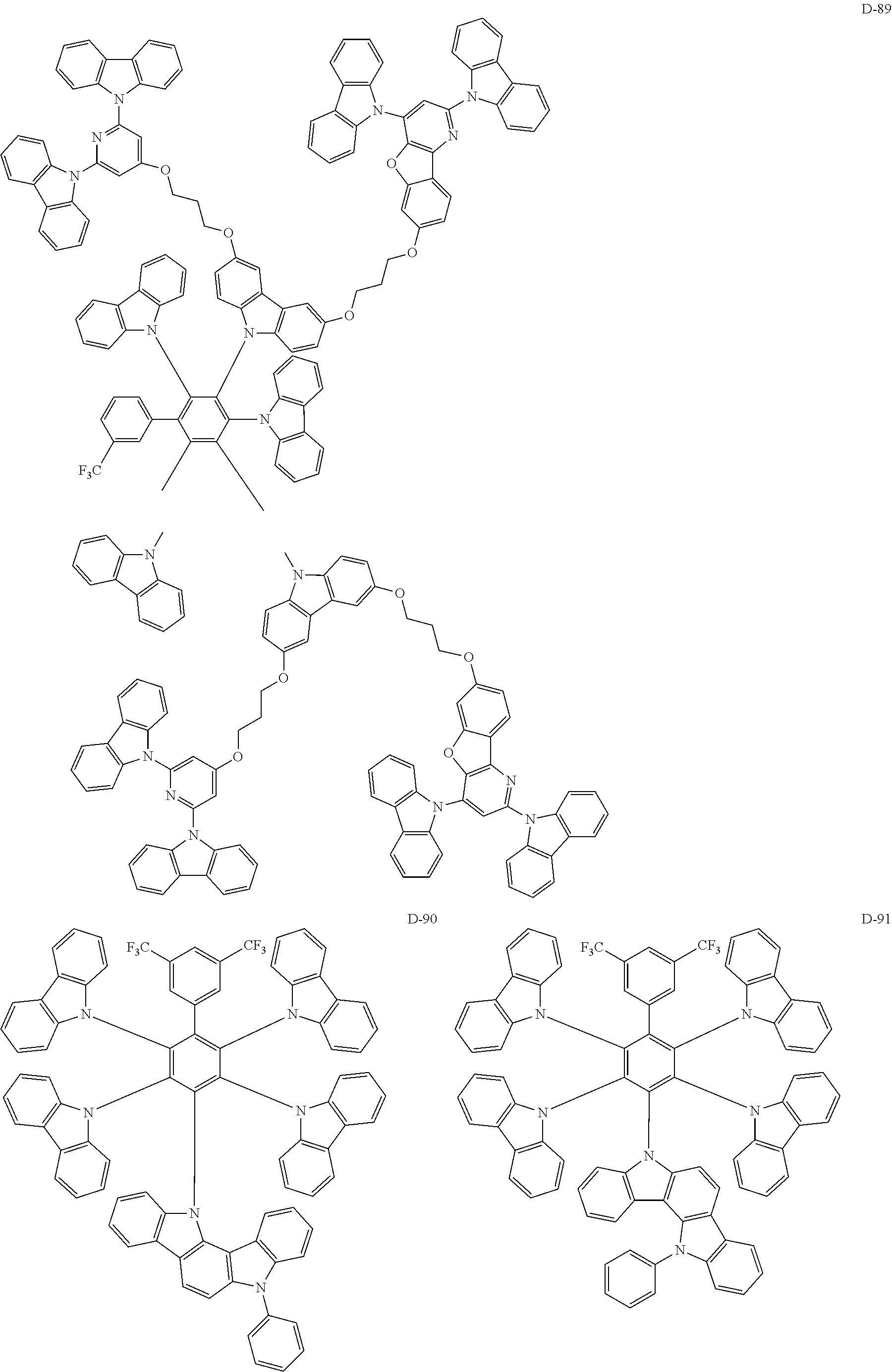

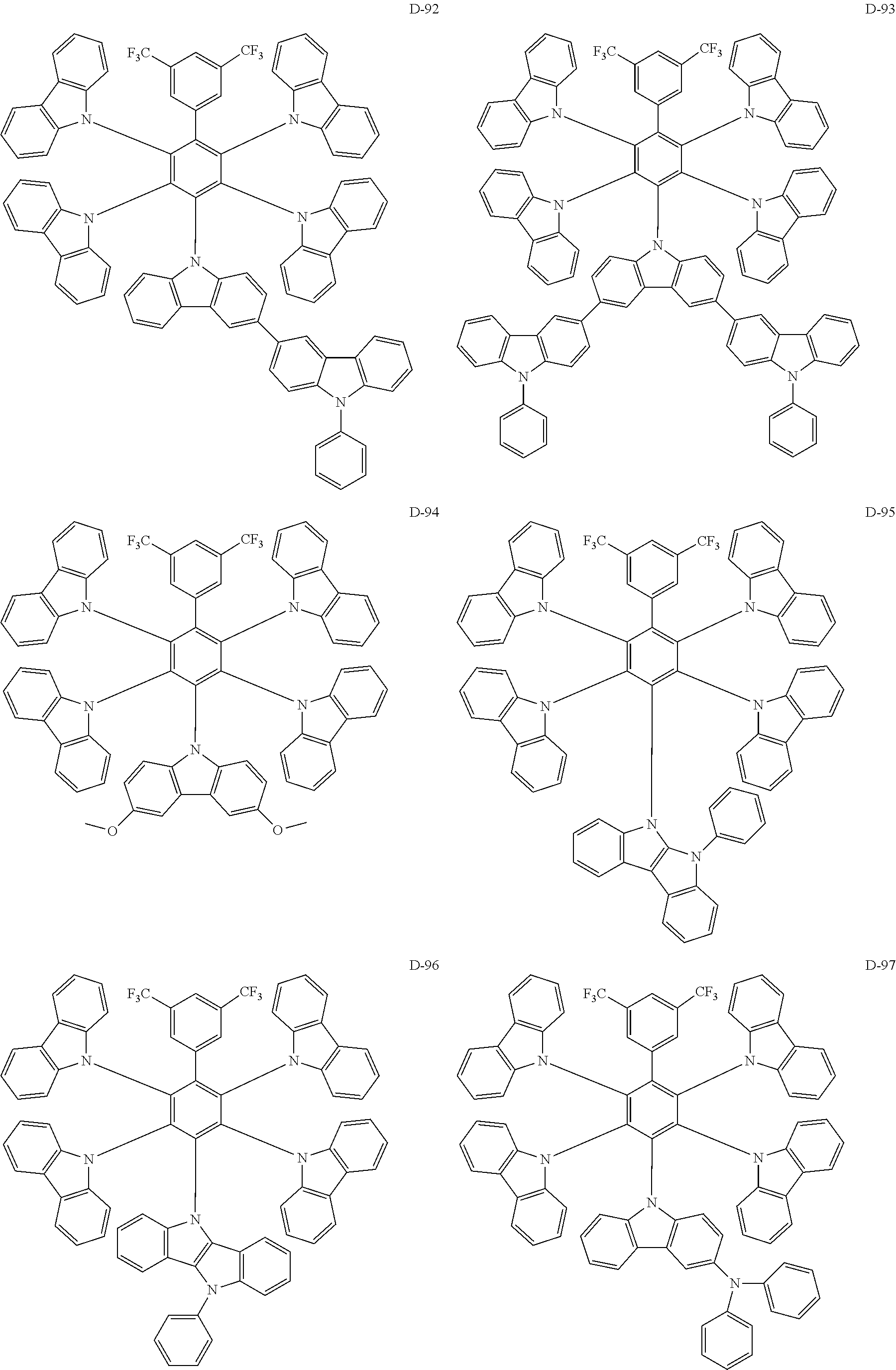

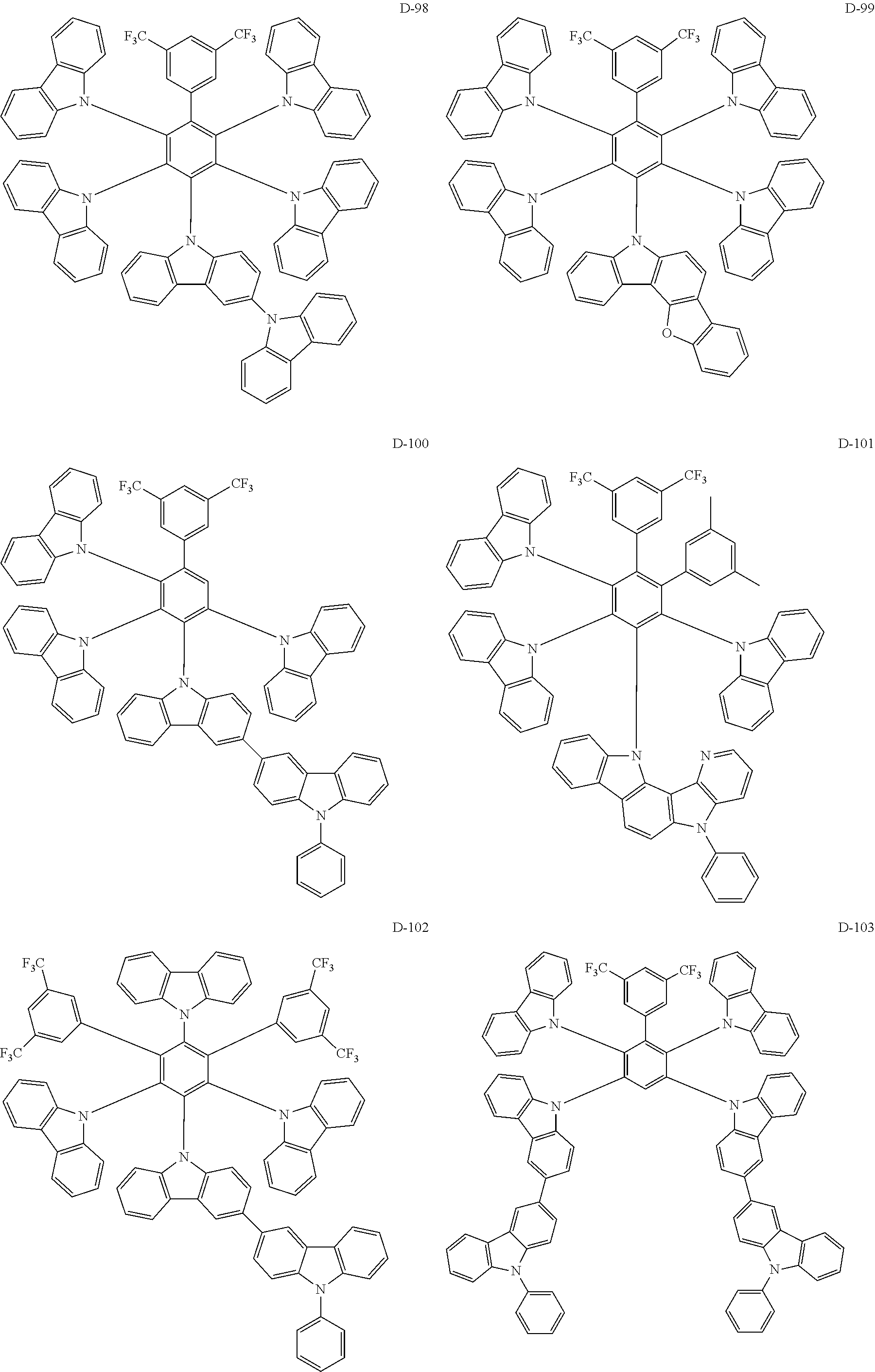

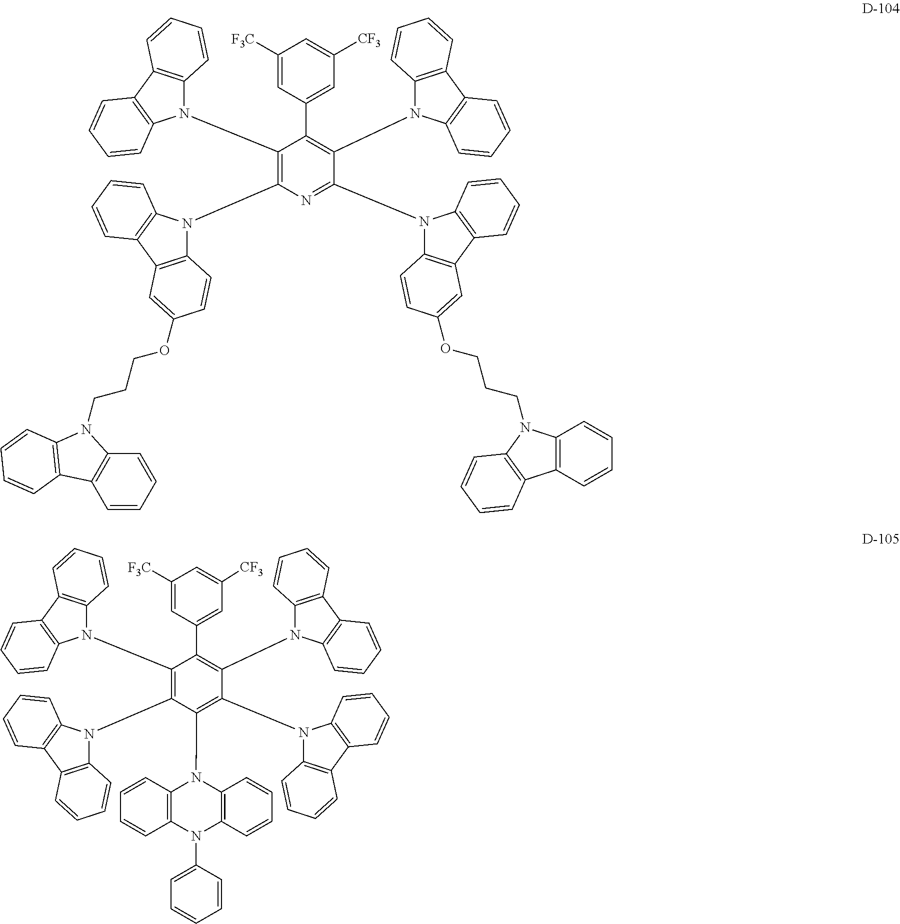 [SMILES: FC(F)(F)c1cc(-c2c(-n3c4ccccc4c4ccccc43)c(-n3c4ccccc4c4cc(OCCCn5c6ccccc6c6ccccc65)ccc43)nc(-n3c4ccccc4c4cc(OCCCn5c6ccccc6c6ccccc65)ccc43)c2-n2c3ccccc3c3ccccc32)cc(C(F)(F)F)c1 ; FC(F)(F)c1cc(-c2c(-n3c4ccccc4c4ccccc43)c(-n3c4ccccc4c4ccccc43)c(N3c4ccccc4N(c4ccccc4)c4ccccc43)c(-n3c4ccccc4c4ccccc43)c2-n2c3ccccc3c3ccccc32)cc(C(F)(F)F)c1]

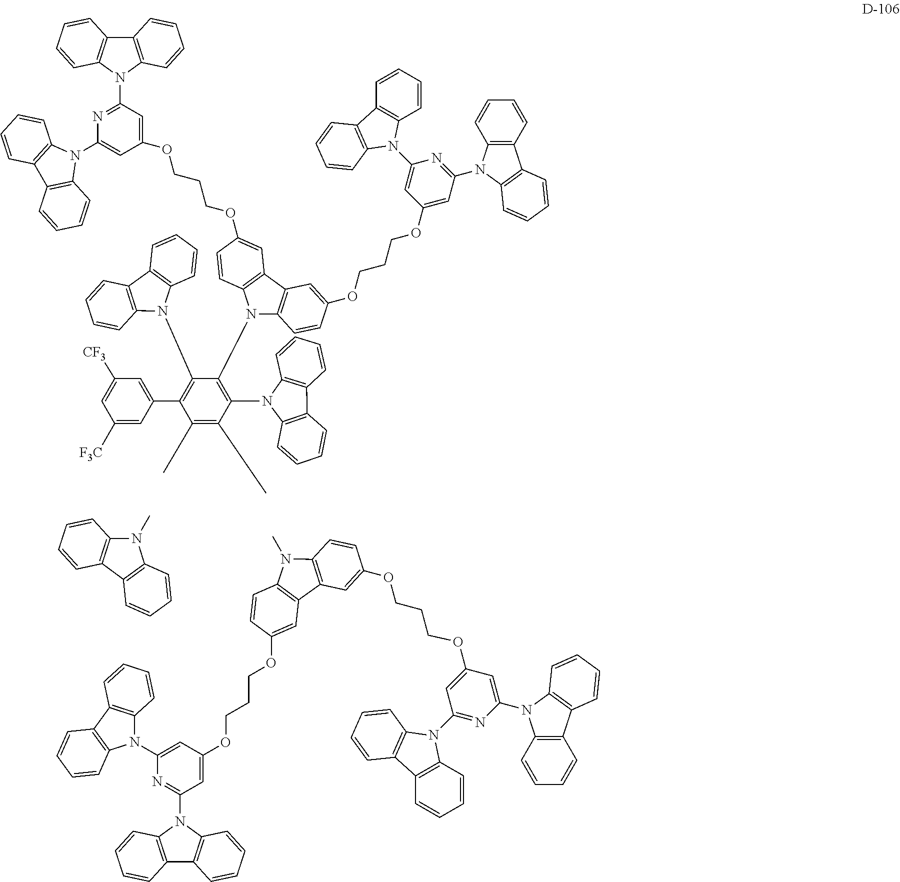 [SMILES: Cc1c(C)c(-n2c3ccccc3c3ccccc32)c(-n2c3ccc(OCCCOc4cc(-n5c6ccccc6c6ccccc65)nc(-n5c6ccccc6c6ccccc65)c4)cc3c3cc(OCCCOc4cc(-n5c6ccccc6c6ccccc65)nc(-n5c6ccccc6c6ccccc65)c4)ccc32)c(-n2c3ccccc3c3ccccc32)c1-c1cc(C(F)(F)F)cc(C(F)(F)F)c1 ; Cn1c2ccc(OCCCOc3cc(-n4c5ccccc5c5ccccc54)nc(-n4c5ccccc5c5ccccc54)c3)cc2c2cc(OCCCOc3cc(-n4c5ccccc5c5ccccc54)nc(-n4c5ccccc5c5ccccc54)c3)ccc21 ; Cn1c2ccccc2c2ccccc21]

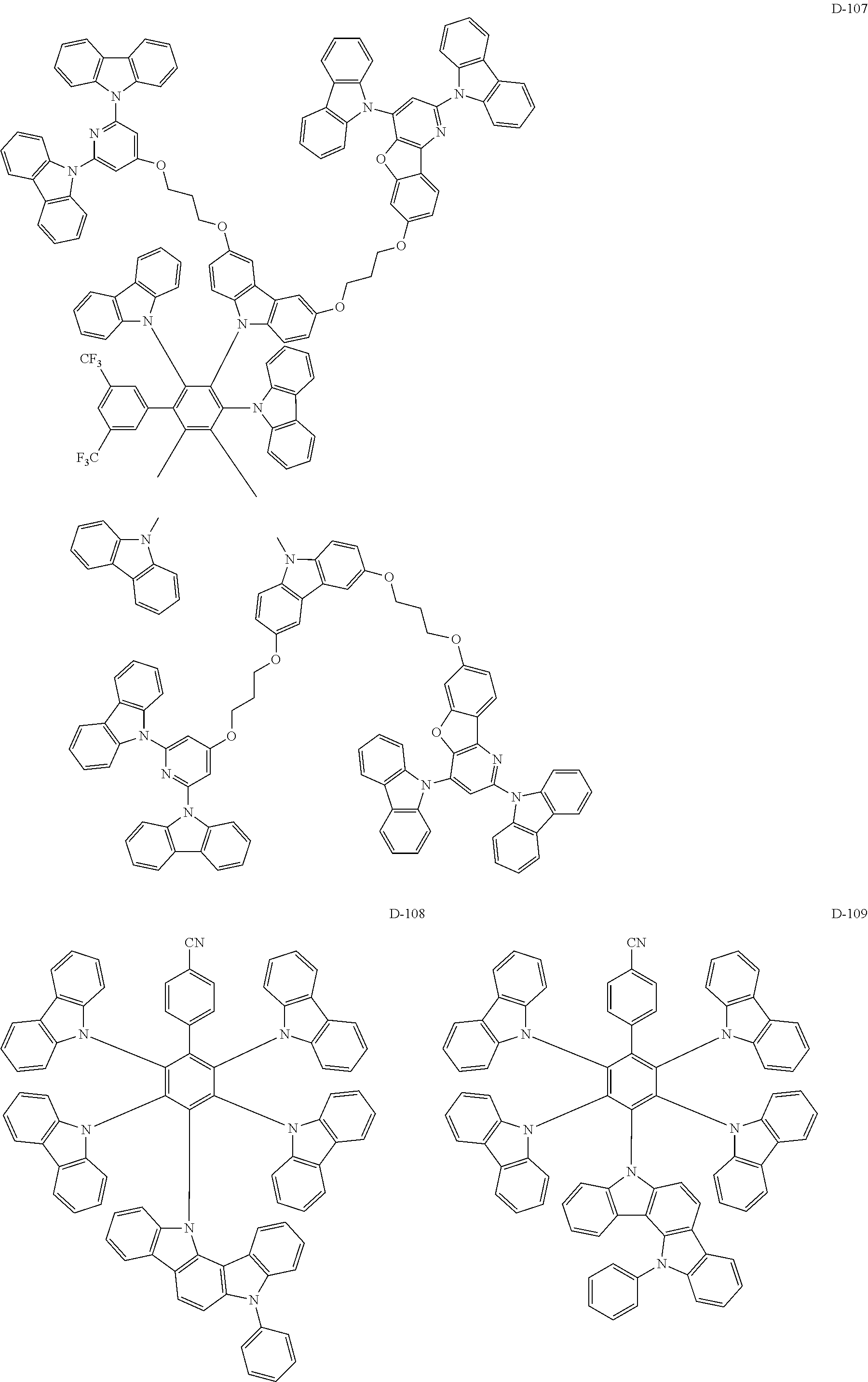

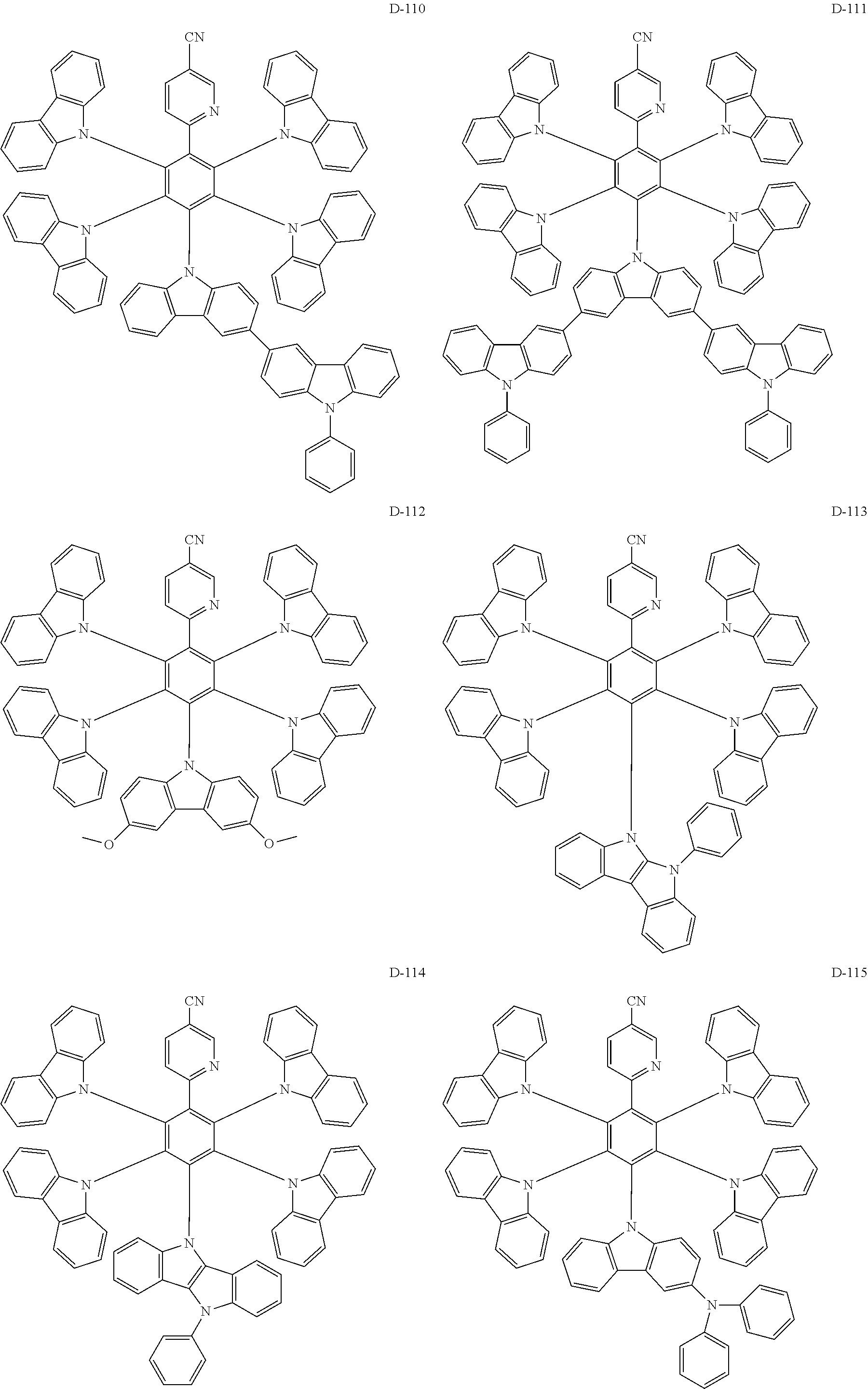

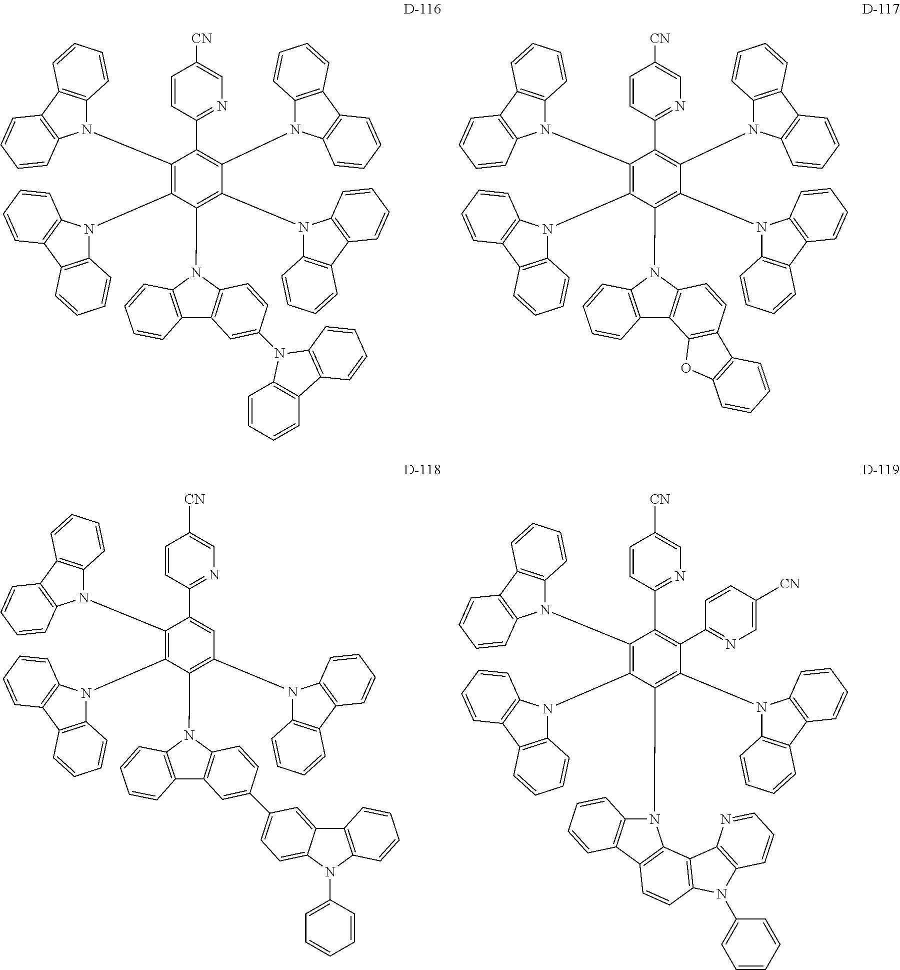

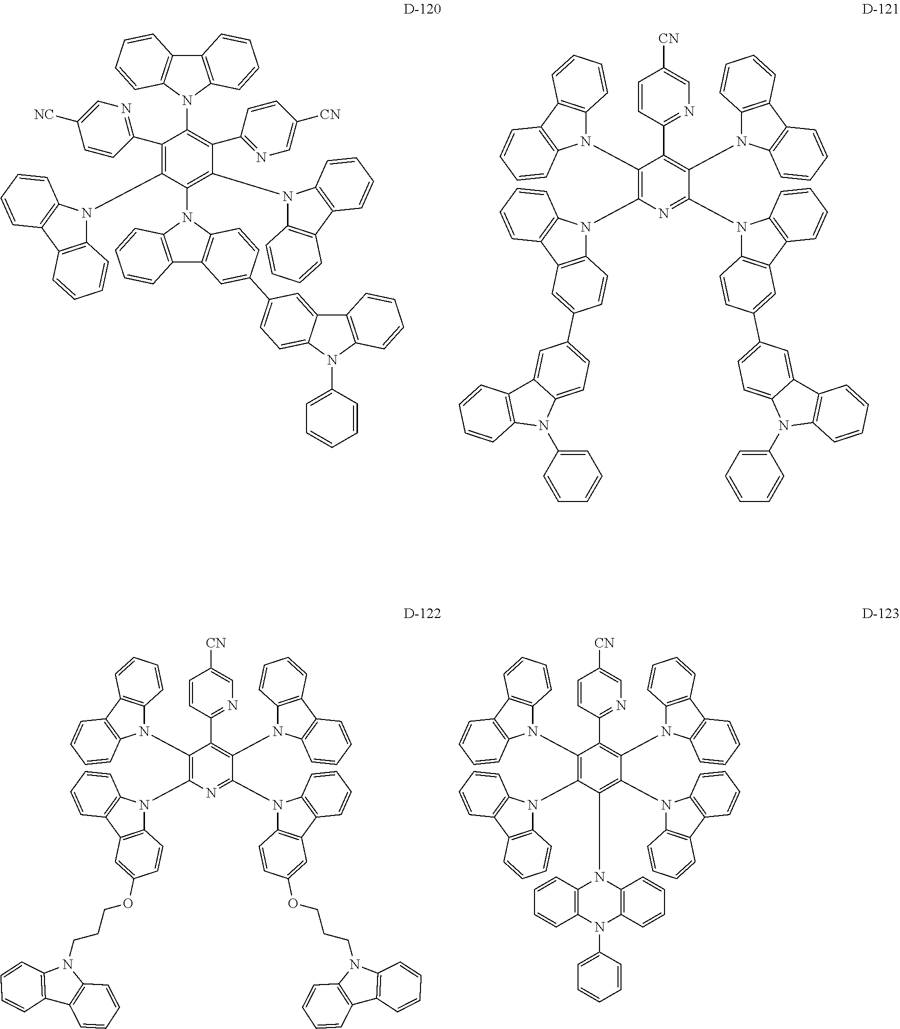

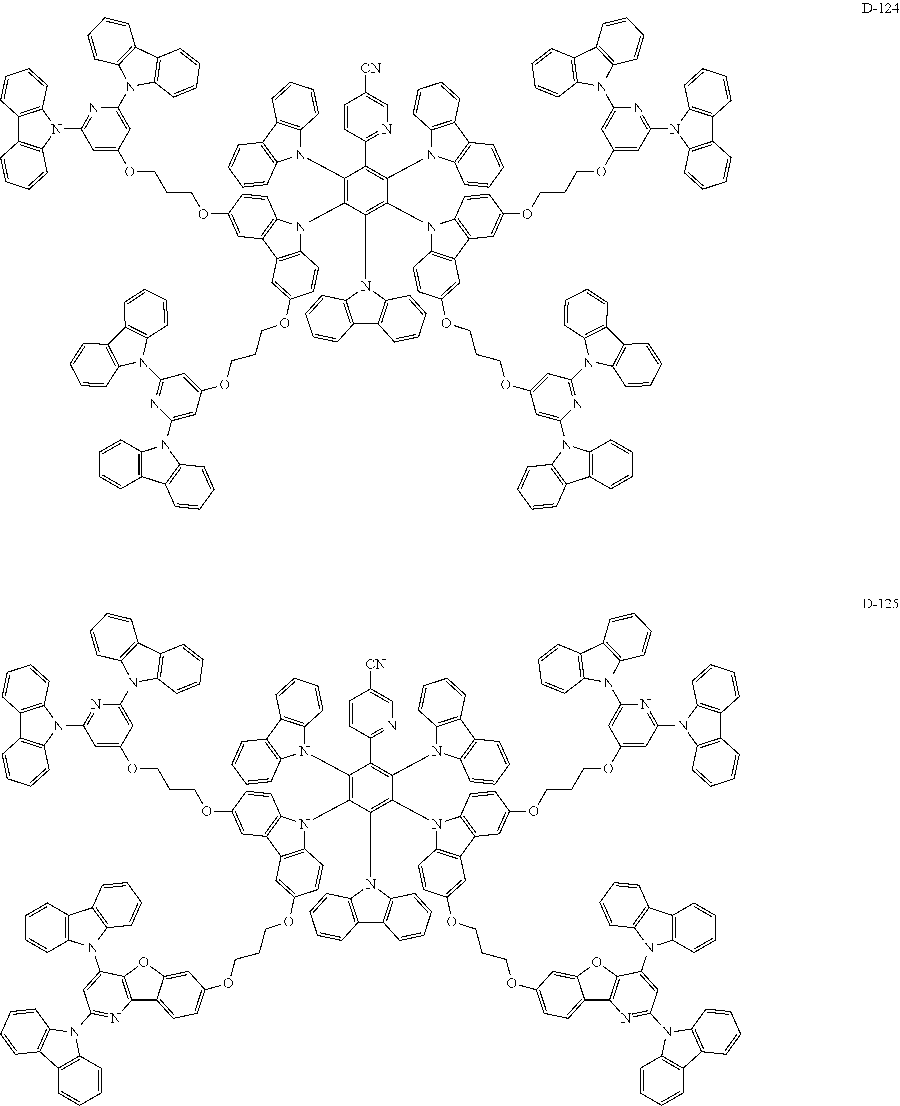

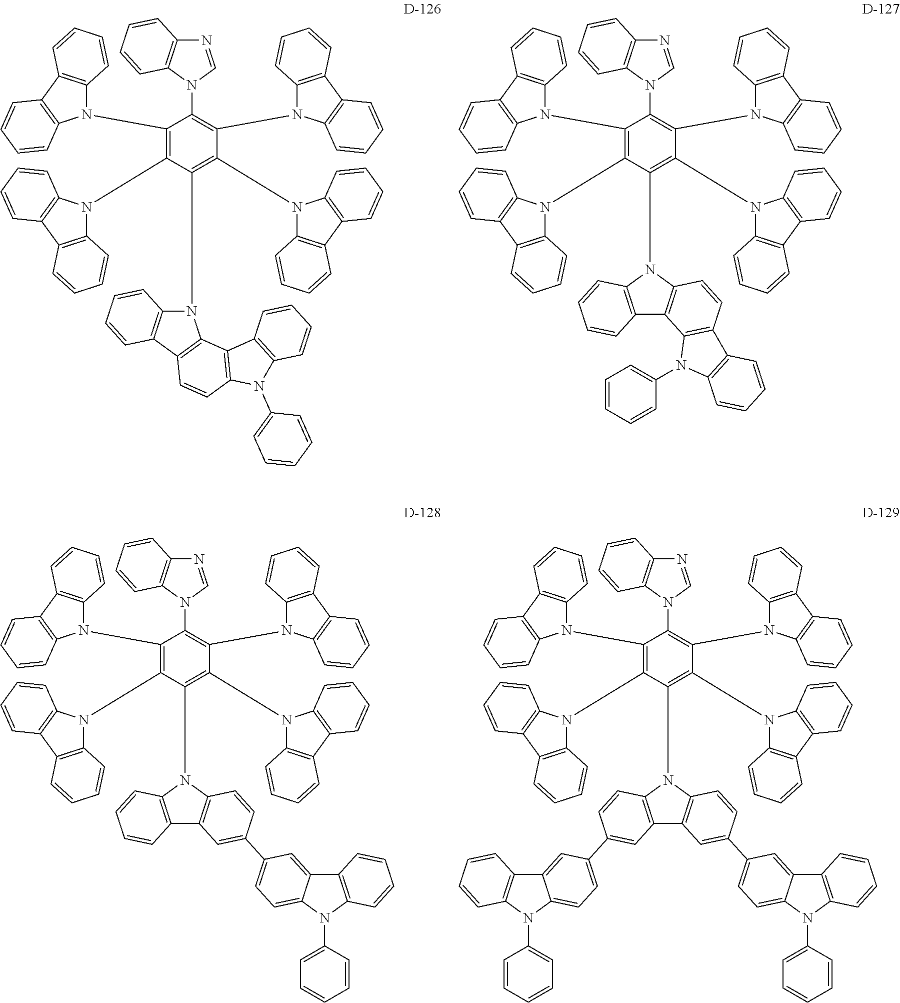

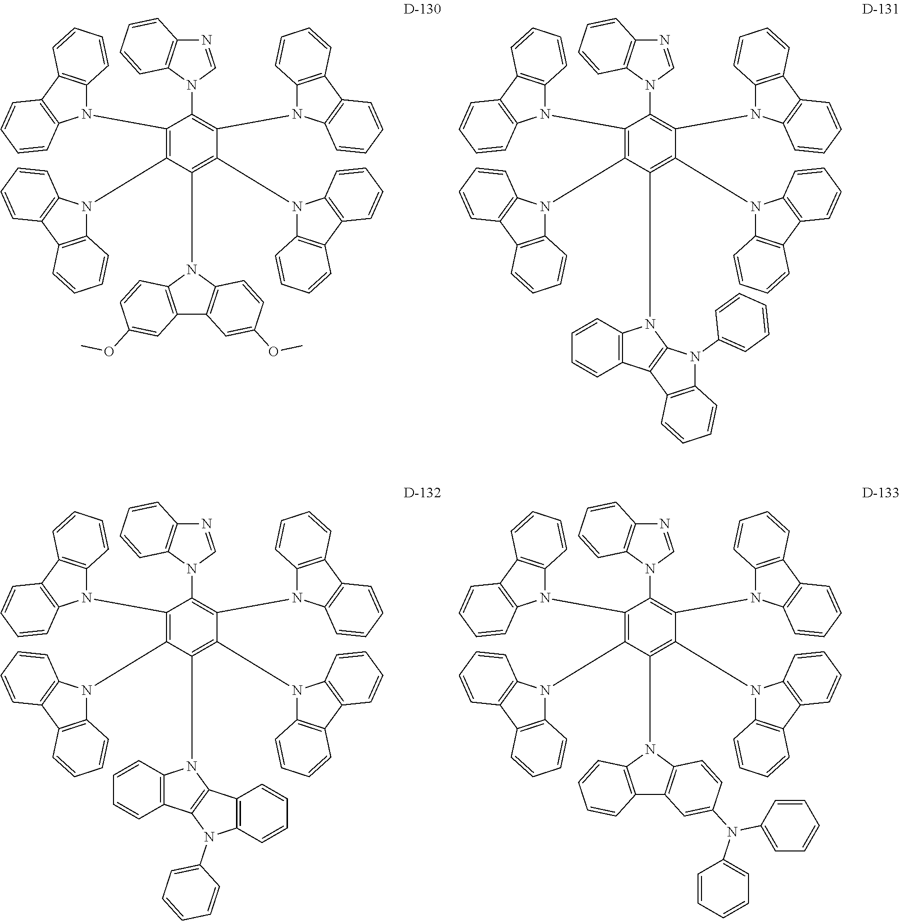

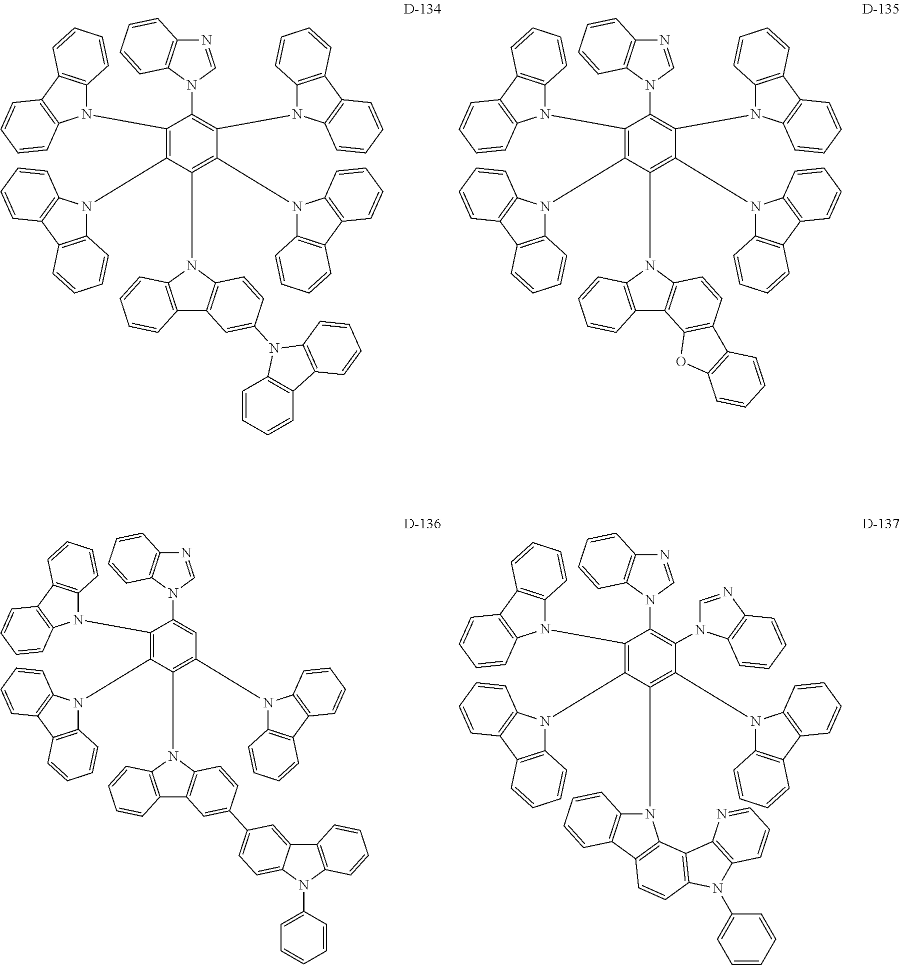

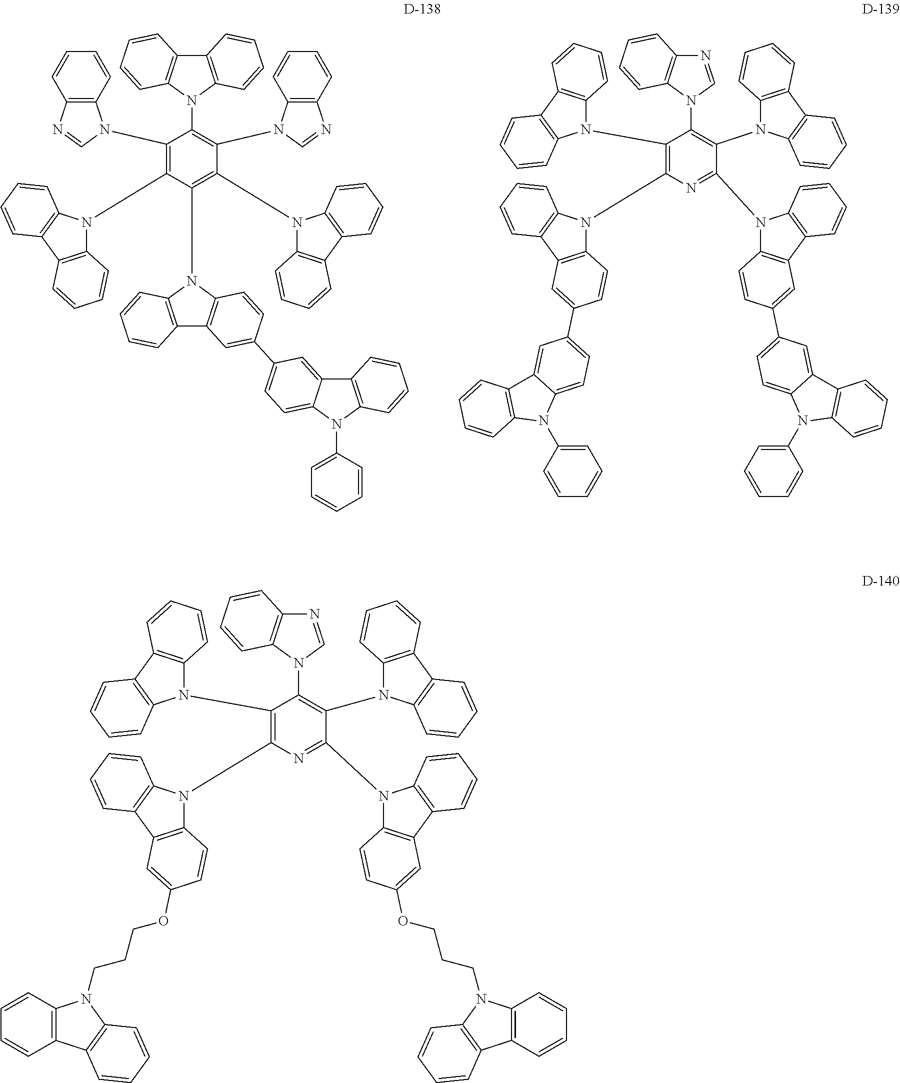

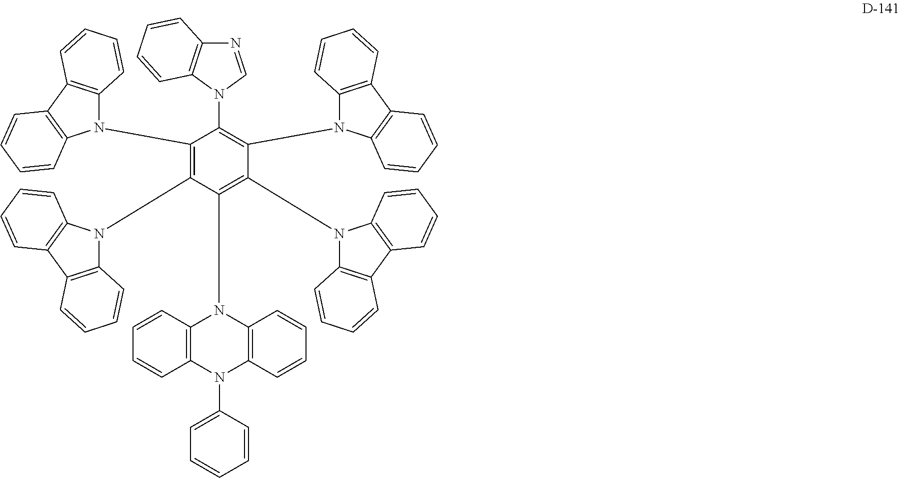 [SMILES: c1ccc(N2c3ccccc3N(c3c(-n4c5ccccc5c5ccccc54)c(-n4c5ccccc5c5ccccc54)c(-n4cnc5ccccc54)c(-n4c5ccccc5c5ccccc54)c3-n3c4ccccc4c4ccccc43)c3ccccc32)cc1]

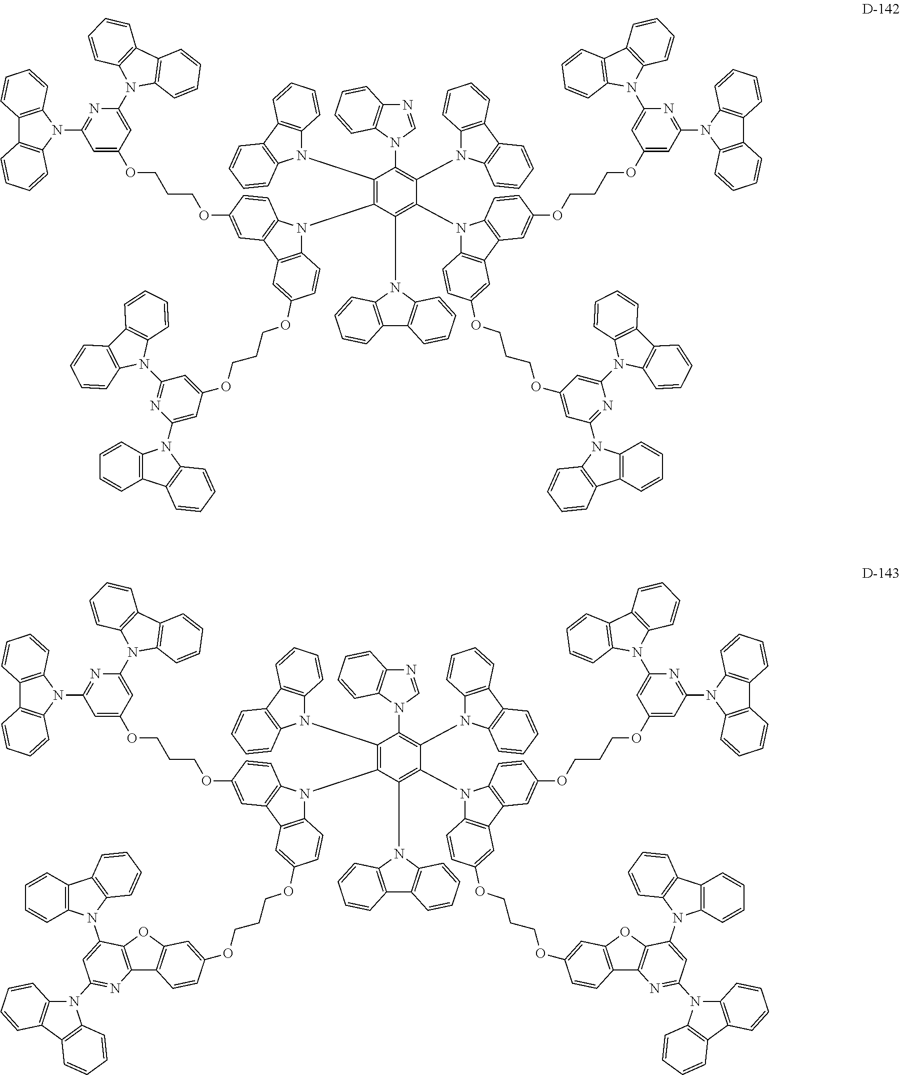

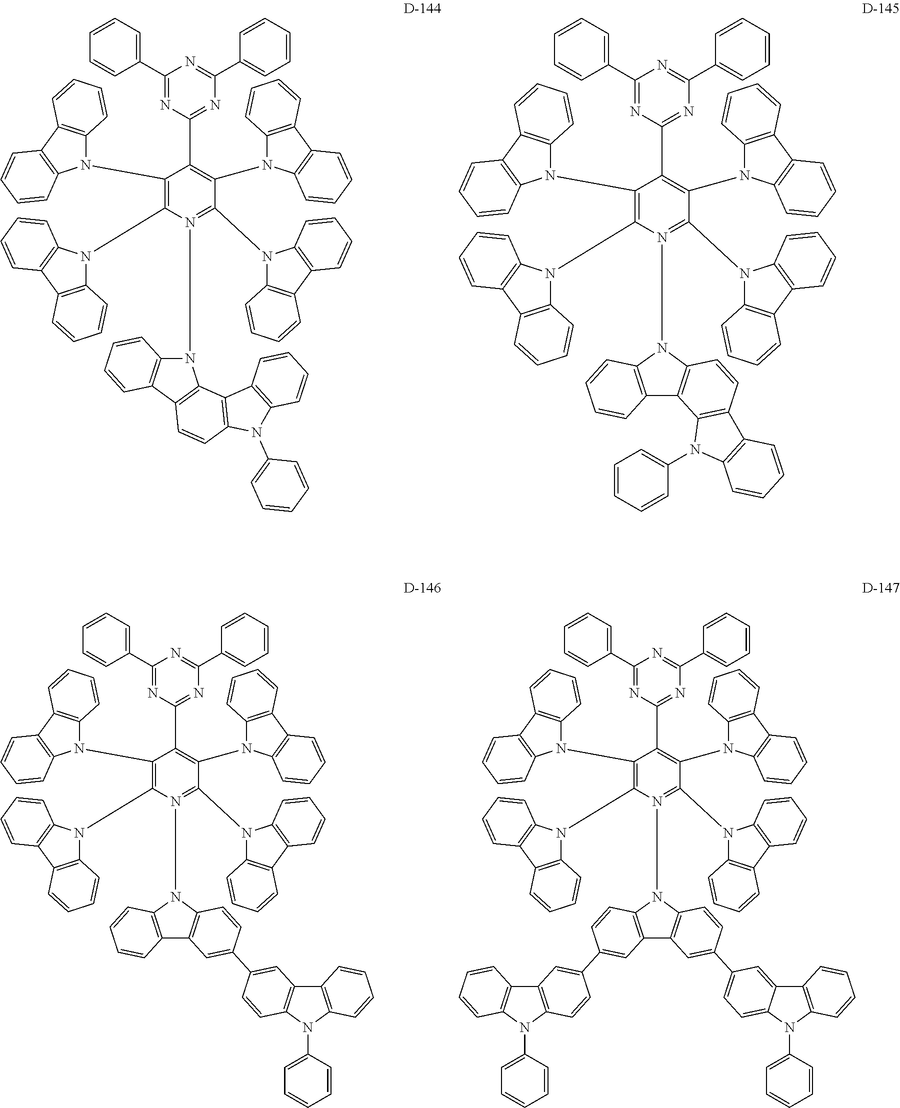

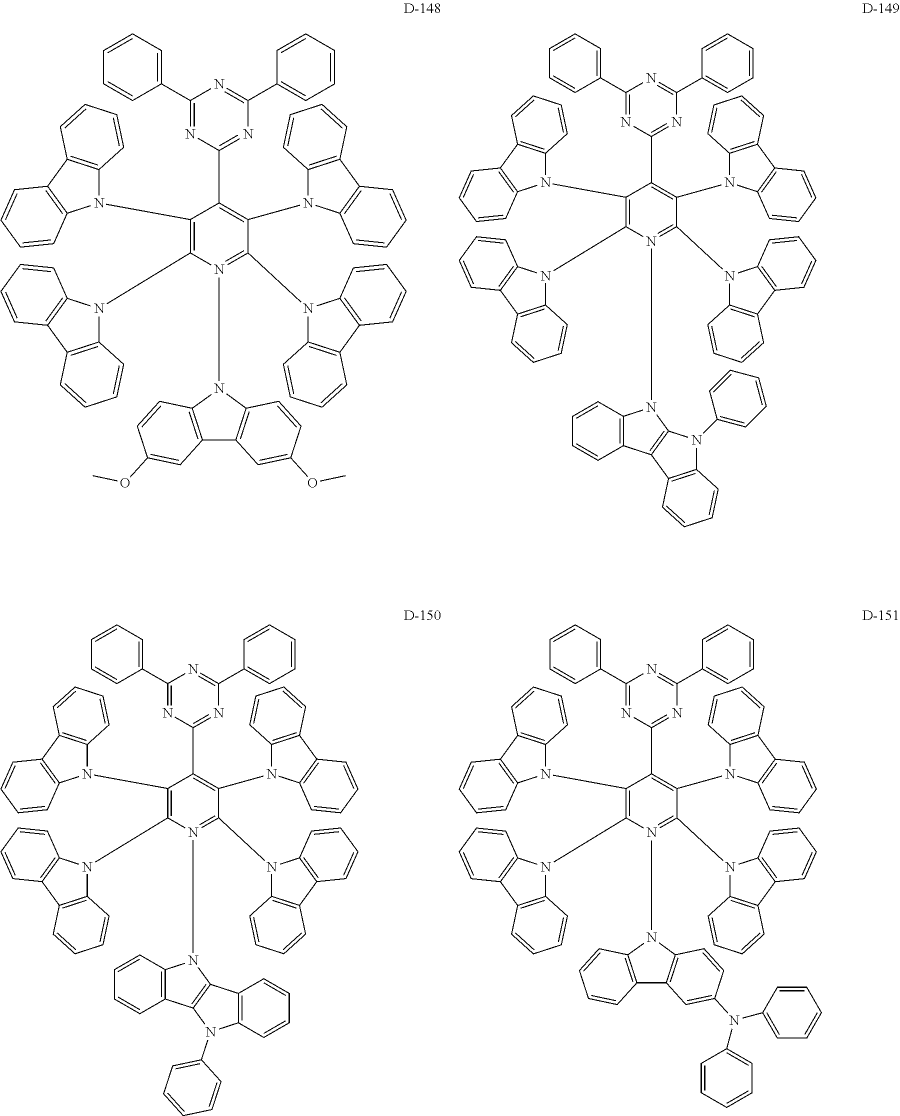

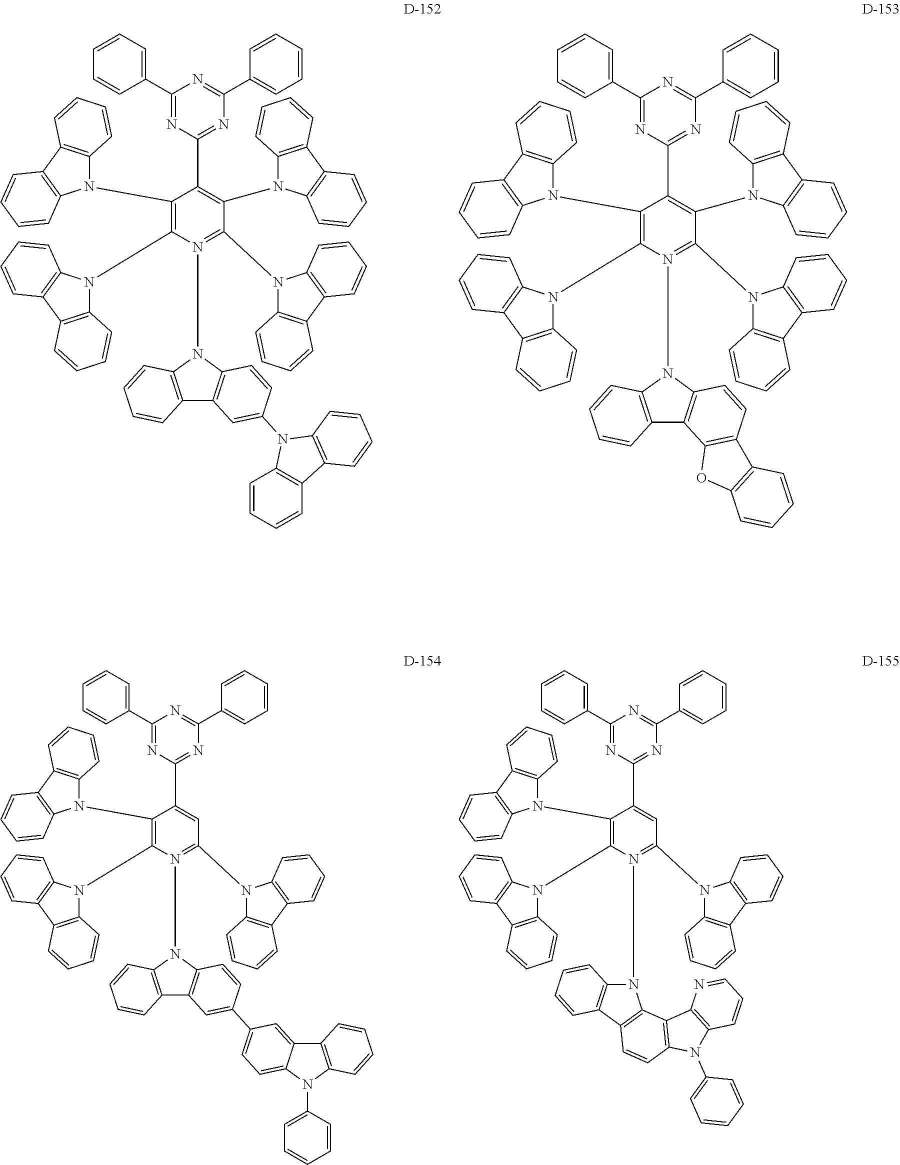

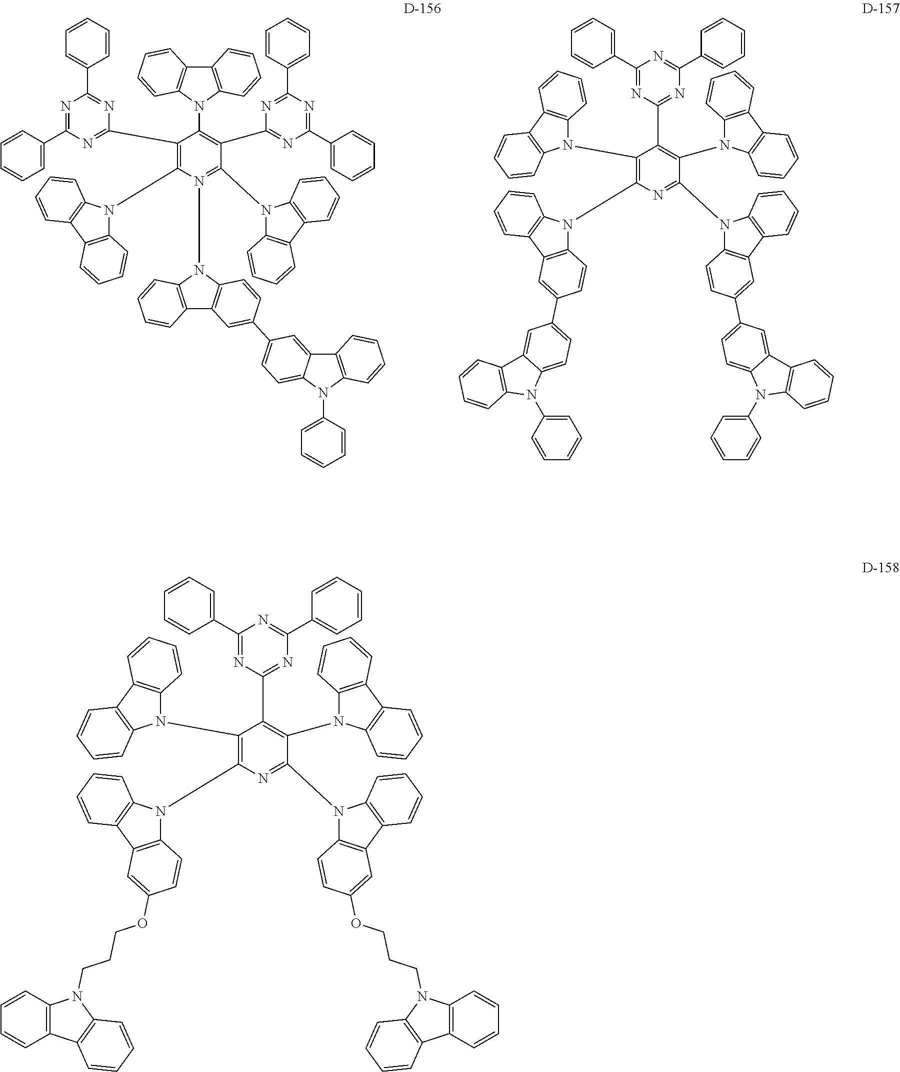

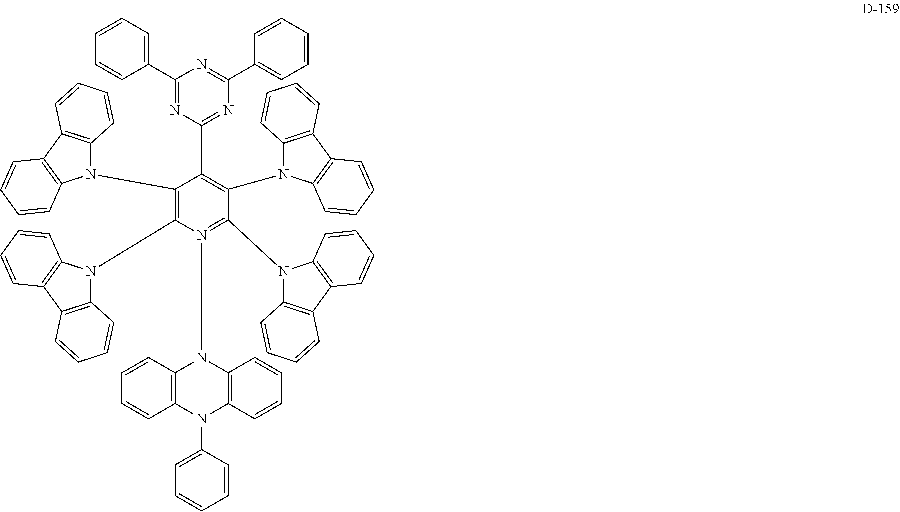

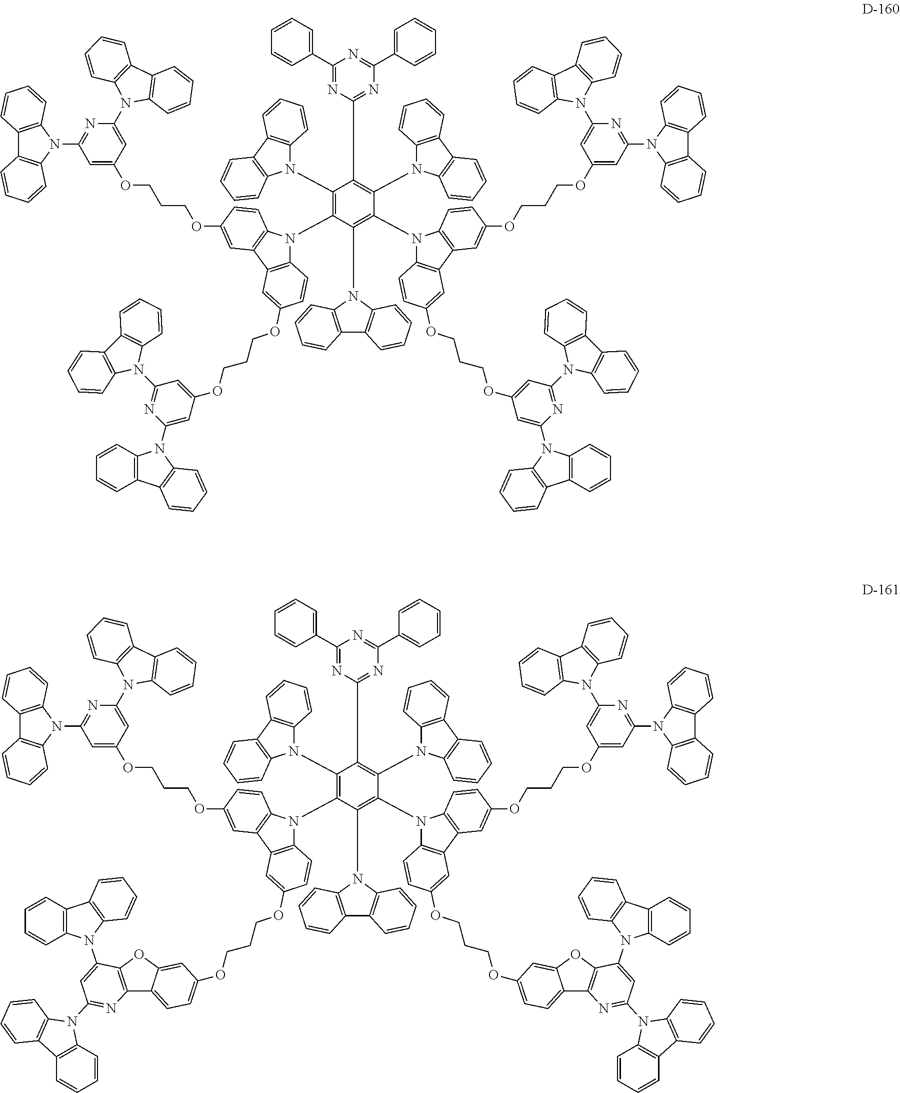

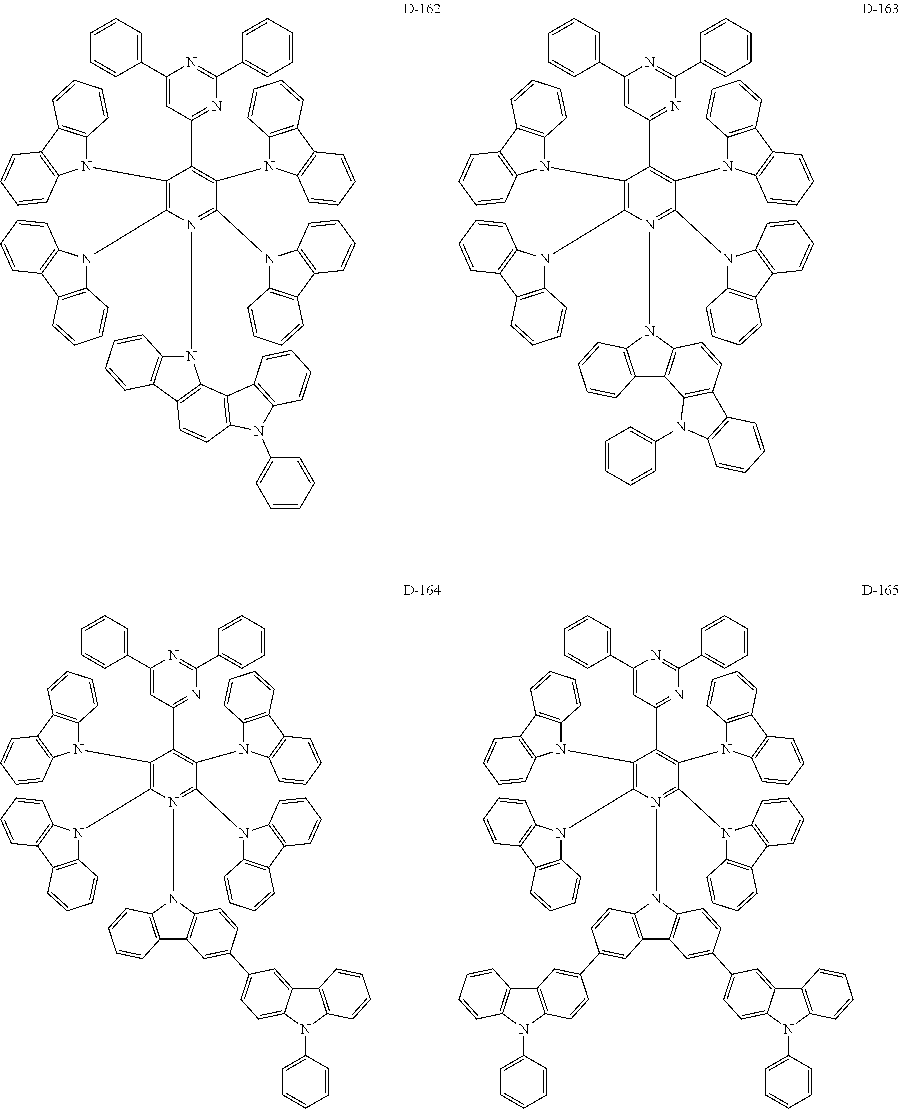

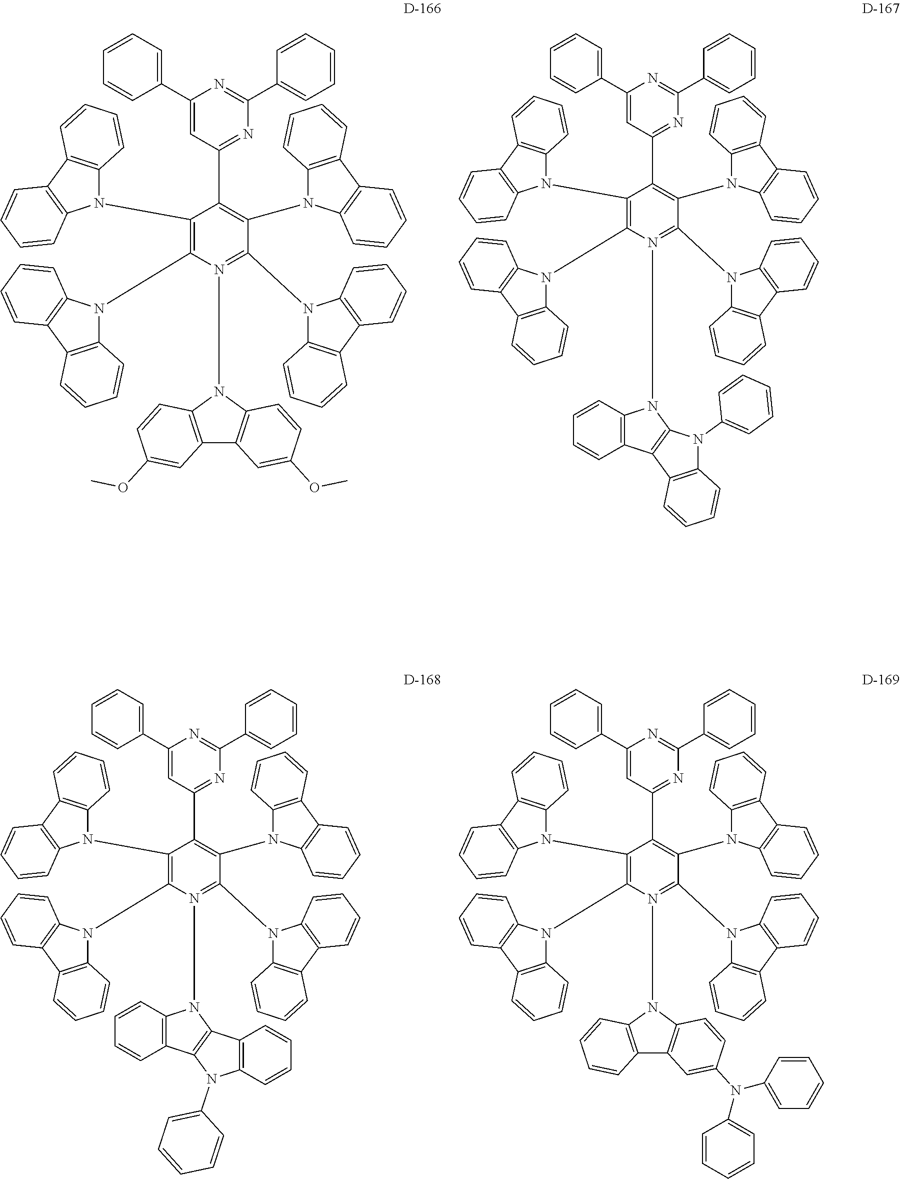

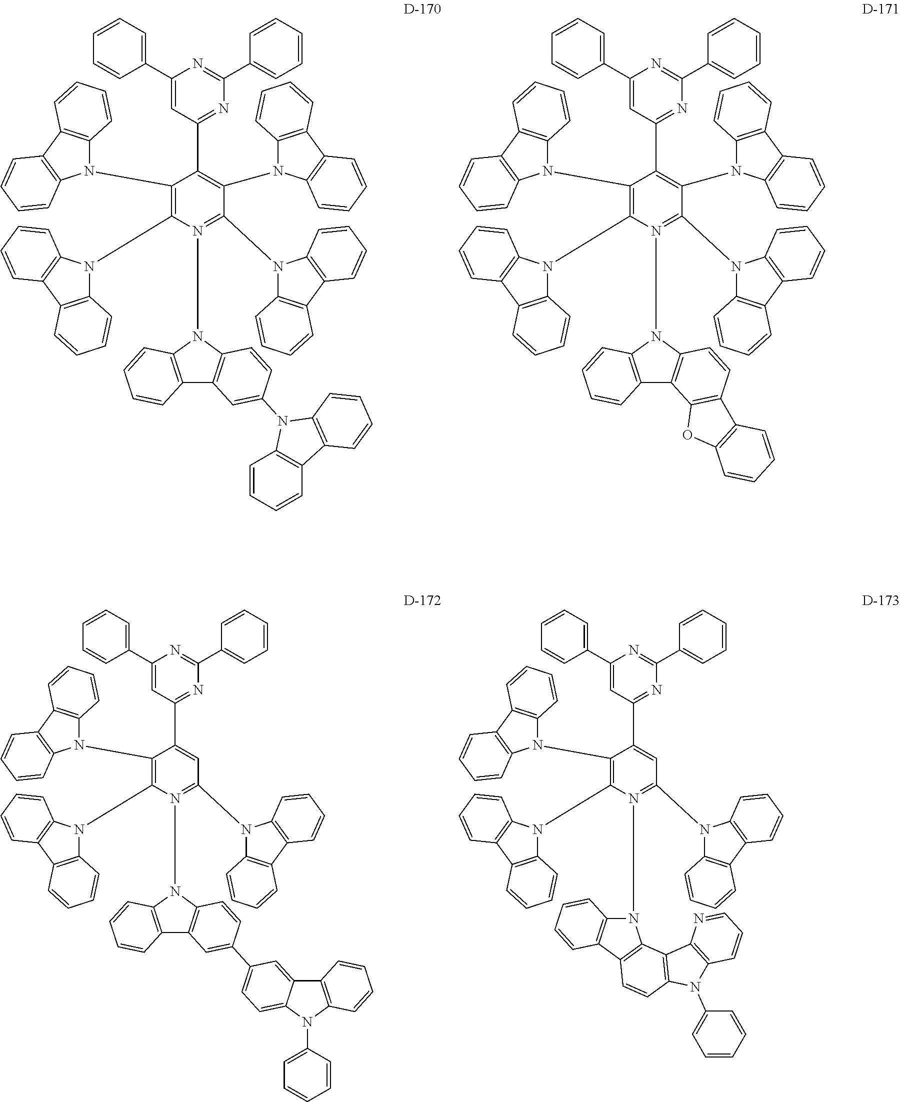

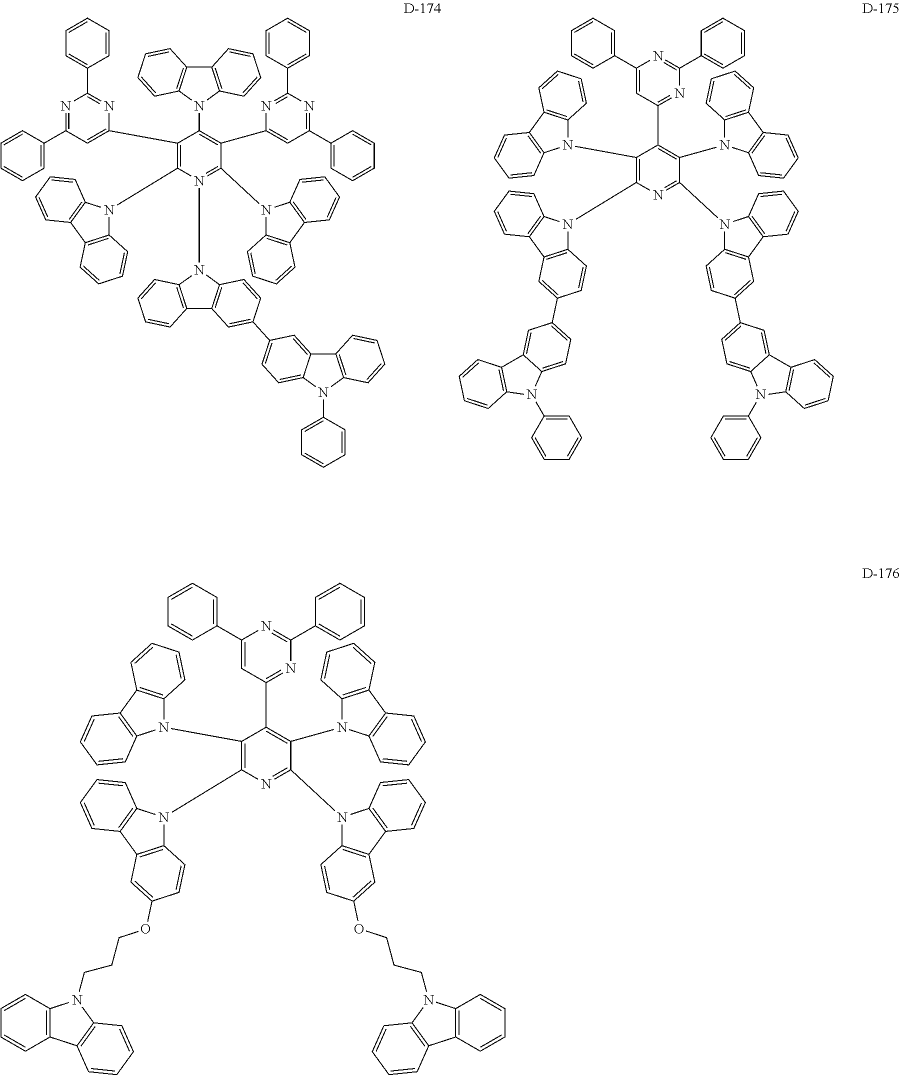

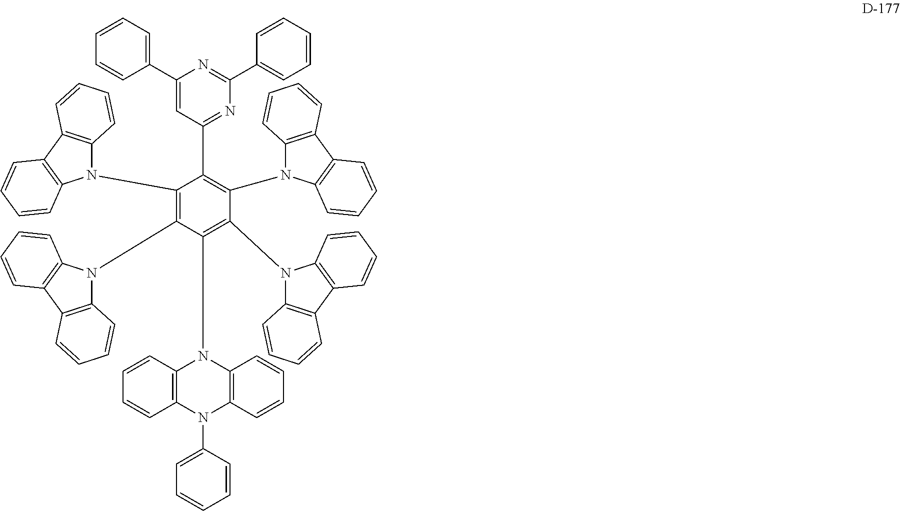 [SMILES: c1ccc(-c2cc(-c3c(-n4c5ccccc5c5ccccc54)c(-n4c5ccccc5c5ccccc54)c(N4c5ccccc5N(c5ccccc5)c5ccccc54)c(-n4c5ccccc5c5ccccc54)c3-n3c4ccccc4c4ccccc43)nc(-c3ccccc3)n2)cc1]

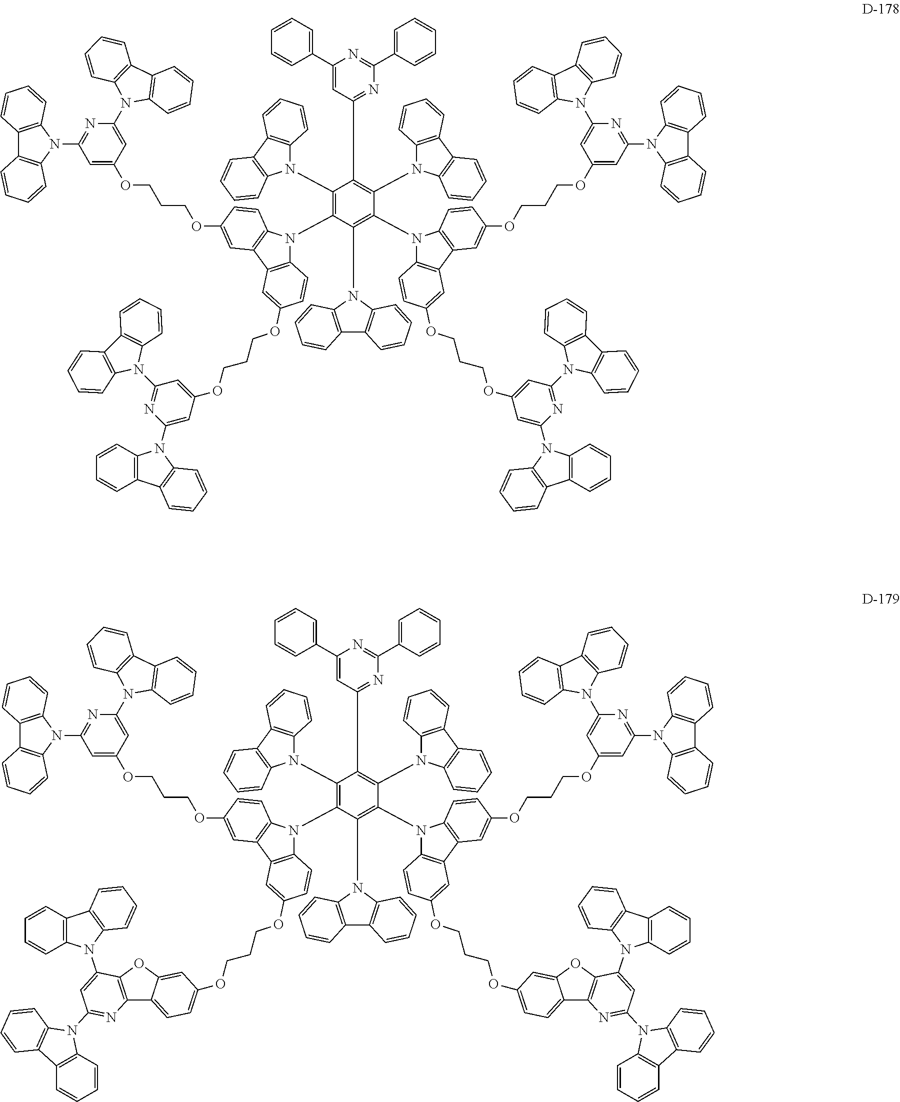

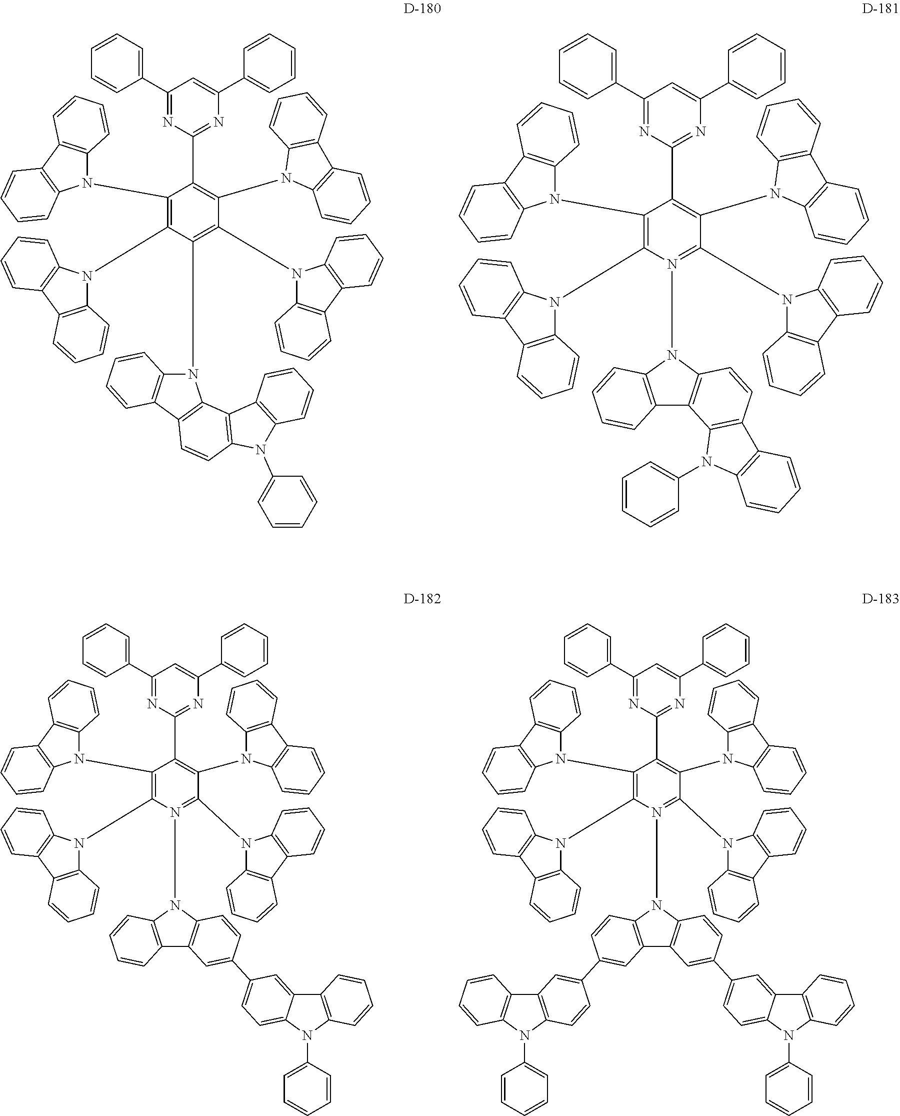

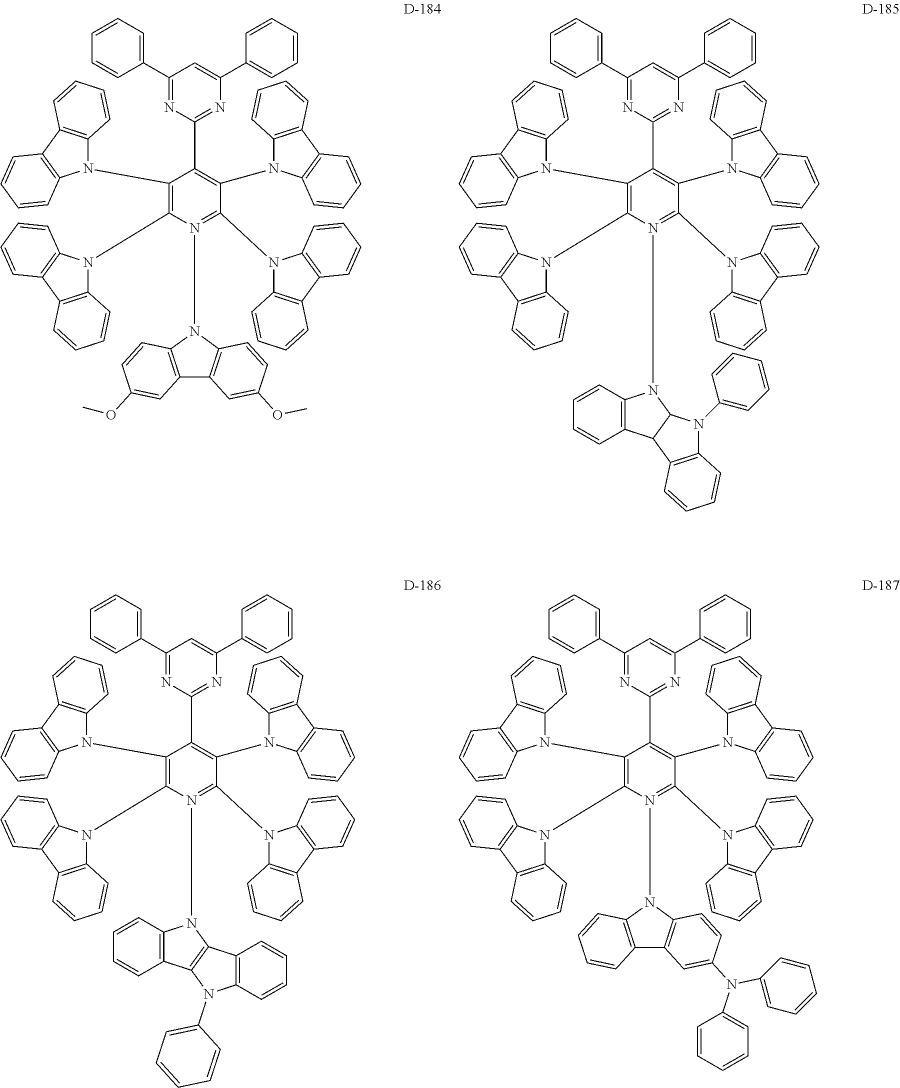

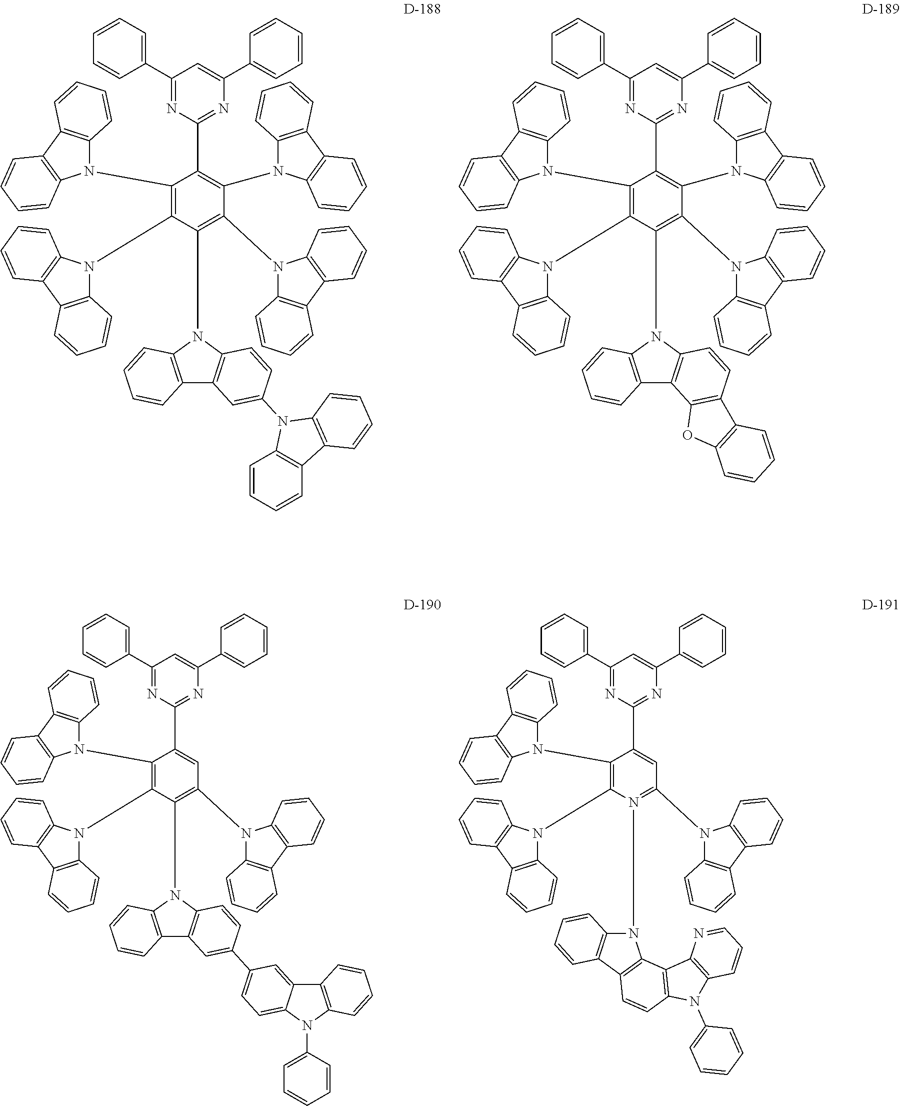

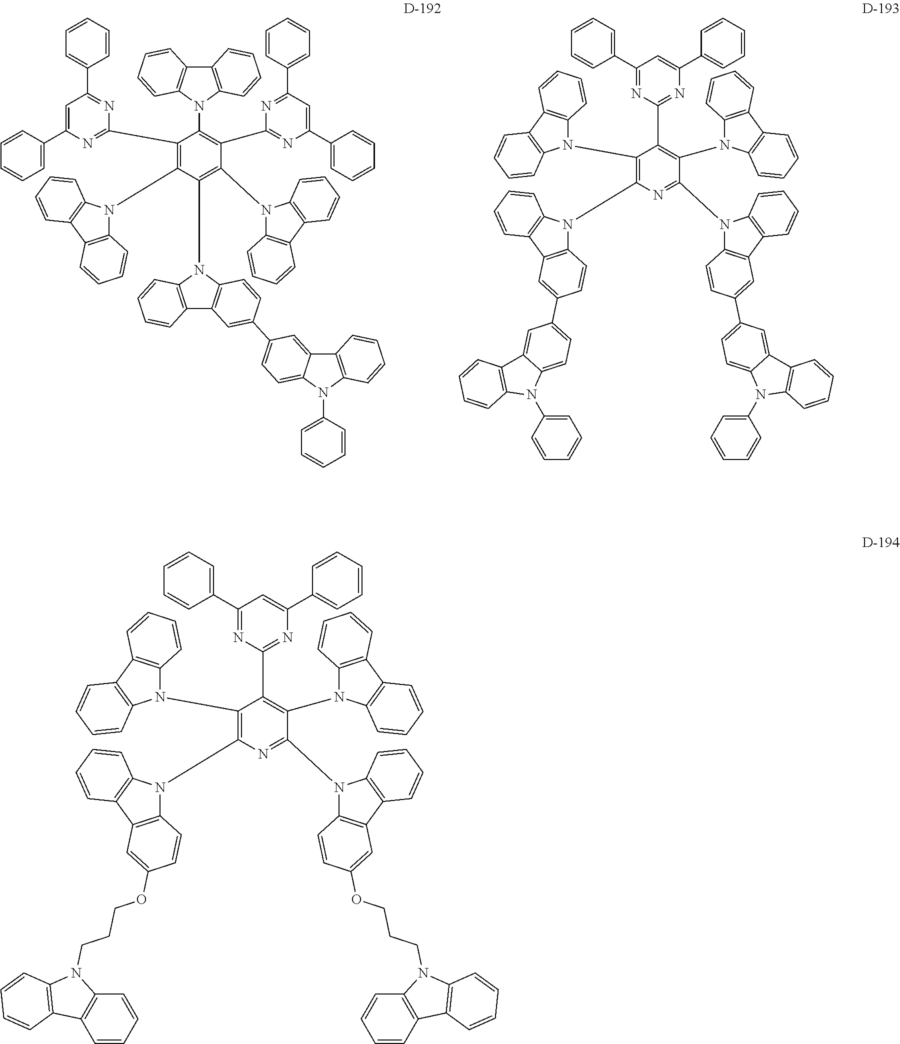

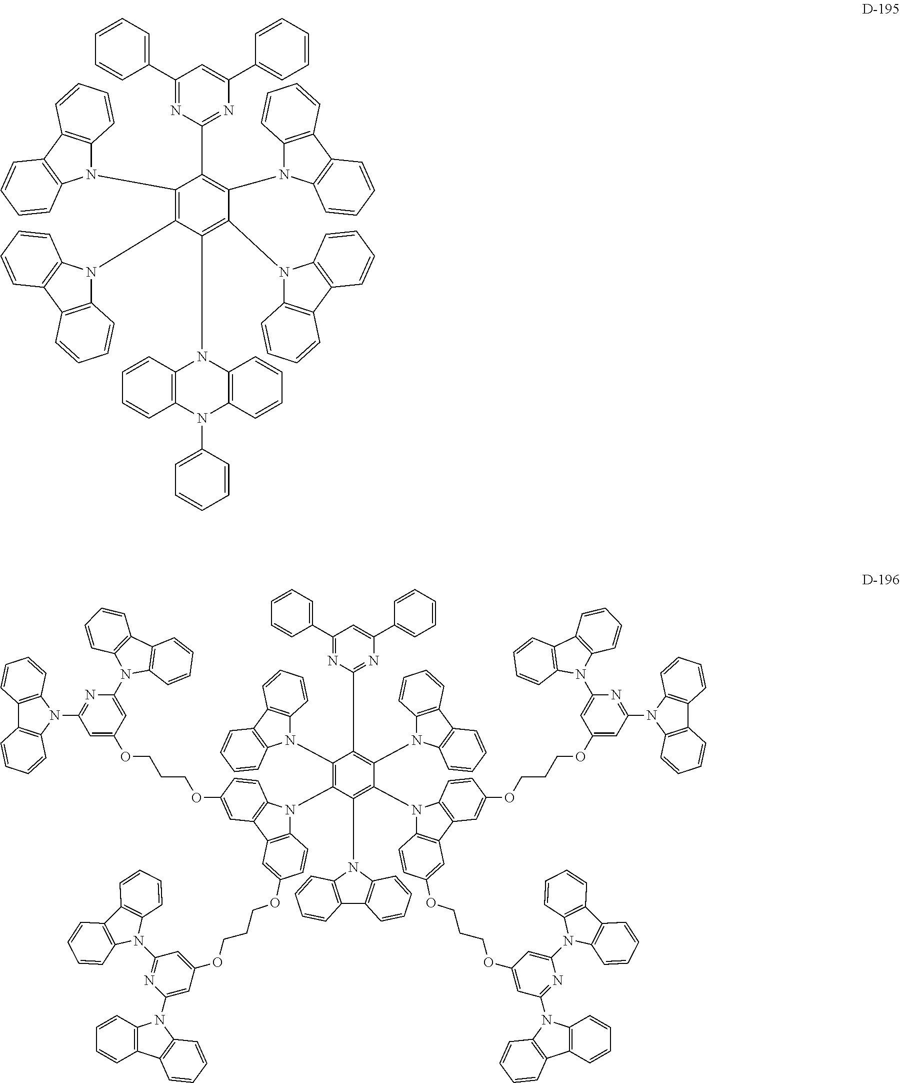

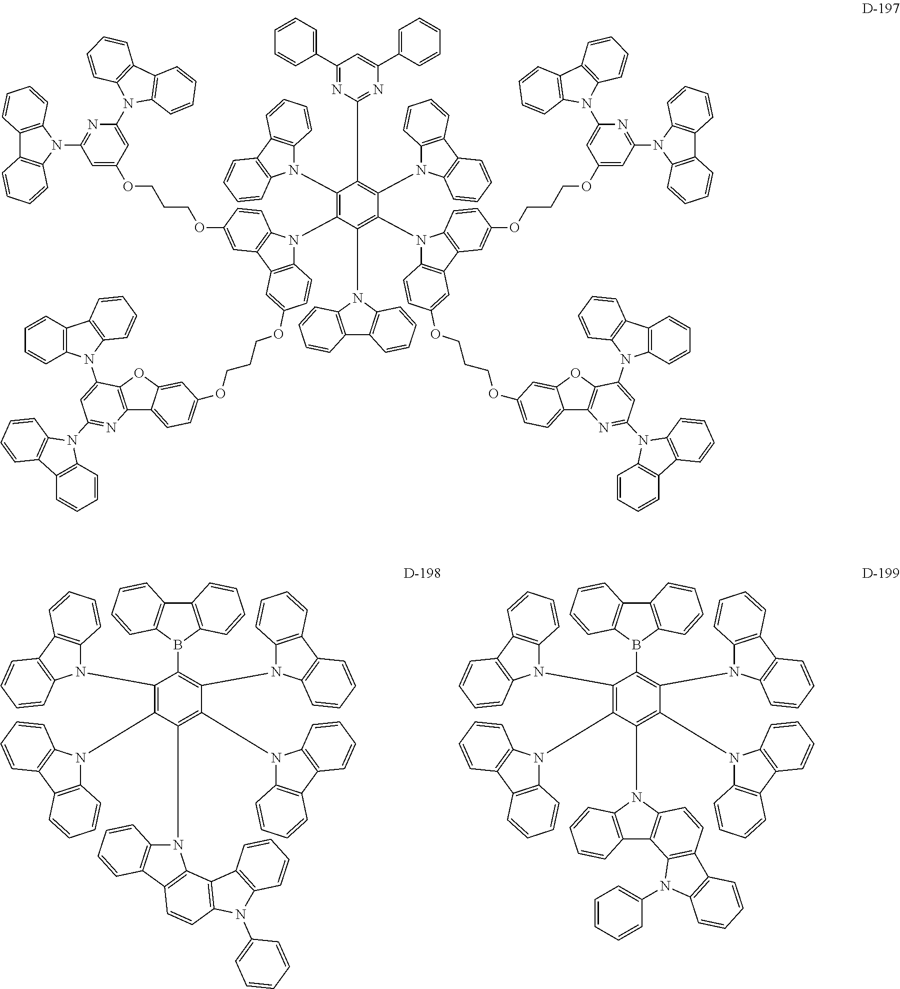

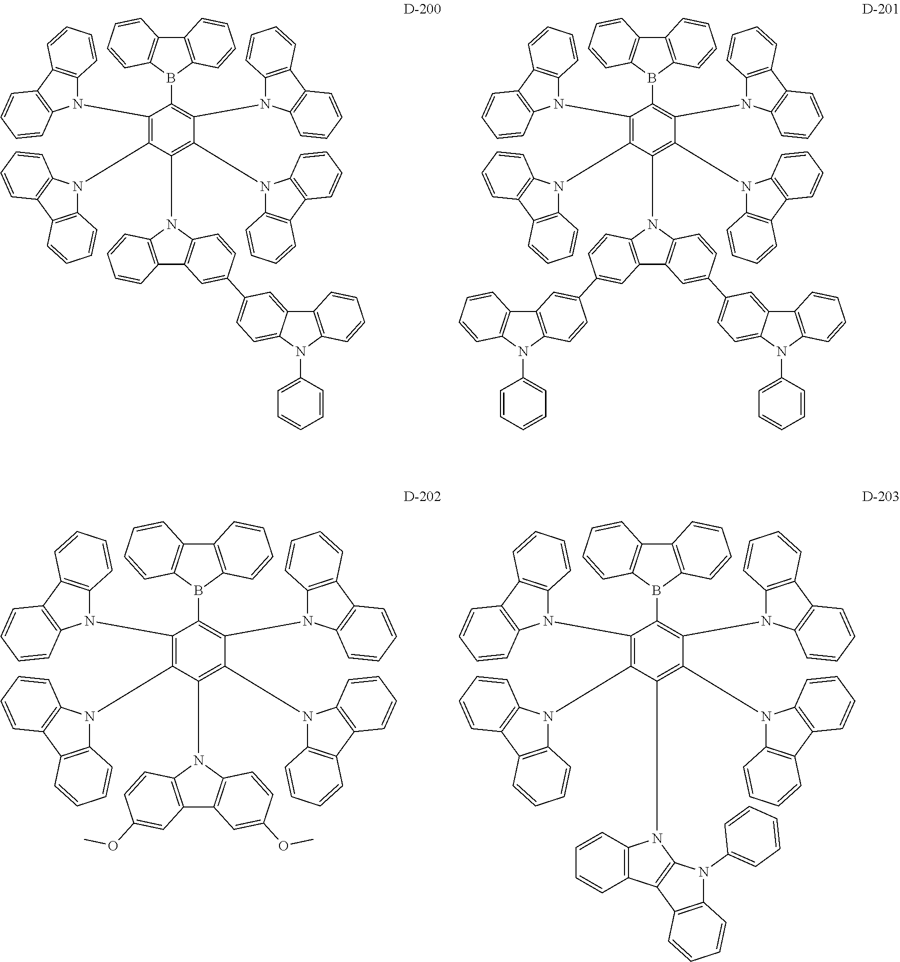

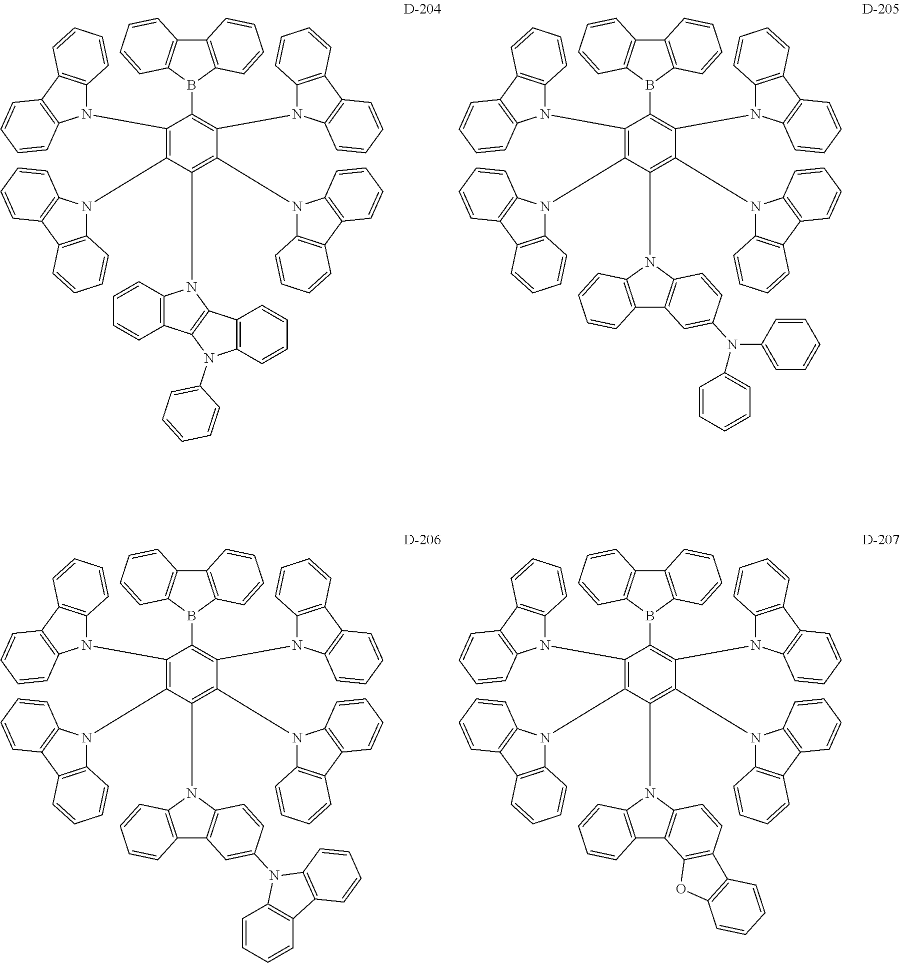

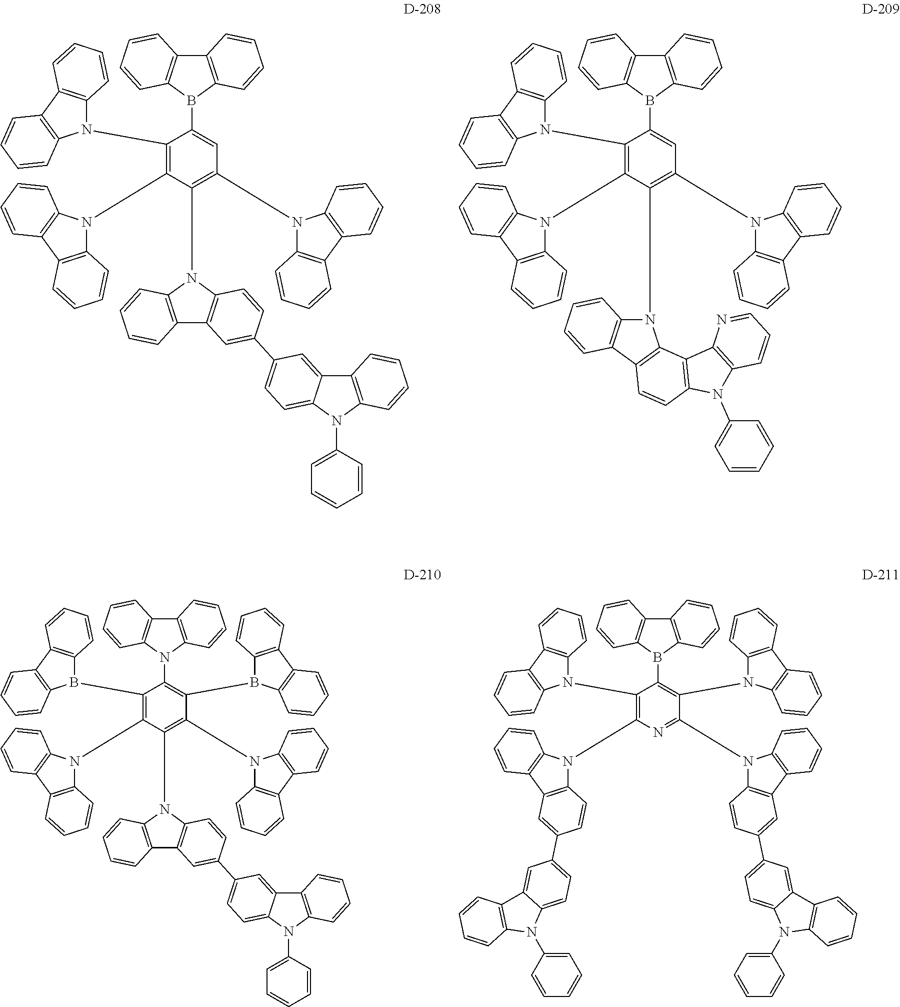

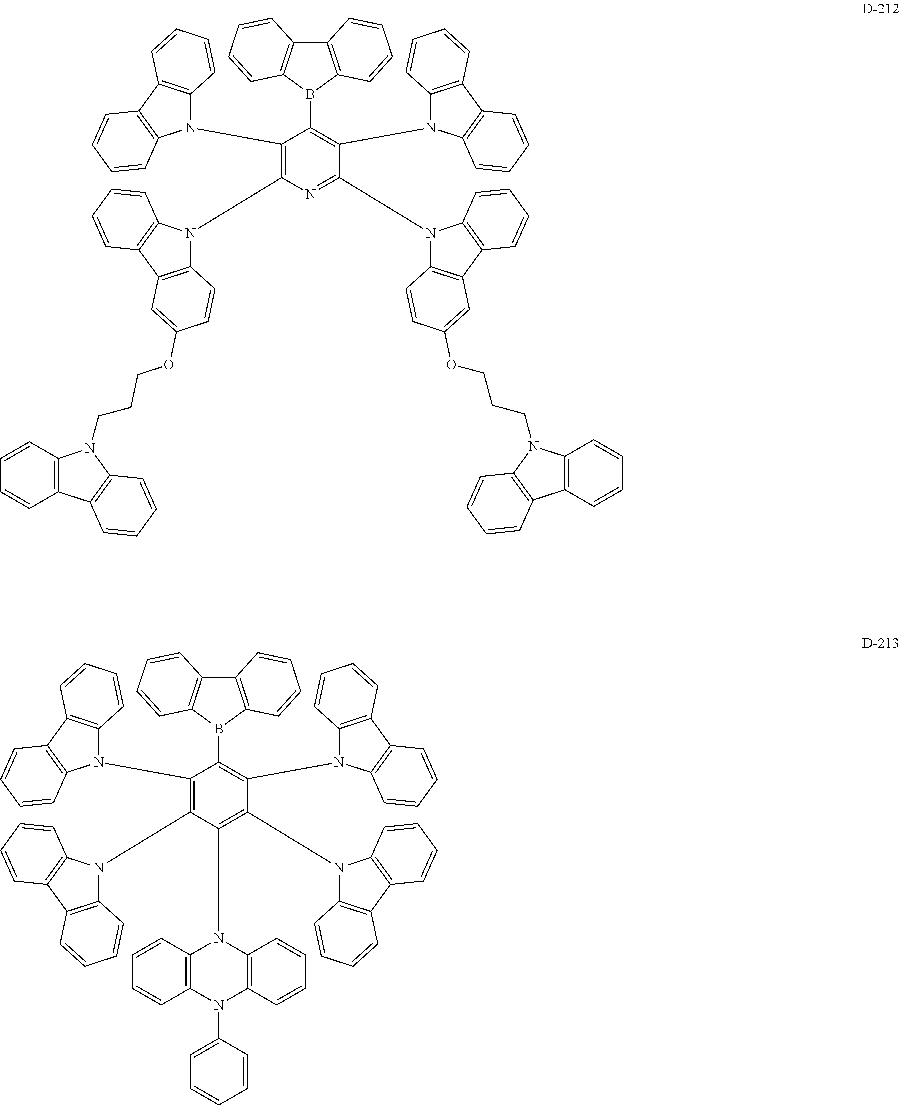 [SMILES: c1ccc2c(c1)B(c1c(-n3c4ccccc4c4ccccc43)c(-n3c4ccccc4c4cc(OCCCn5c6ccccc6c6ccccc65)ccc43)nc(-n3c4ccccc4c4cc(OCCCn5c6ccccc6c6ccccc65)ccc43)c1-n1c3ccccc3c3ccccc31)c1ccccc1-2 ; c1ccc(N2c3ccccc3N(c3c(-n4c5ccccc5c5ccccc54)c(-n4c5ccccc5c5ccccc54)c(B4c5ccccc5-c5ccccc54)c(-n4c5ccccc5c5ccccc54)c3-n3c4ccccc4c4ccccc43)c3ccccc32)cc1]

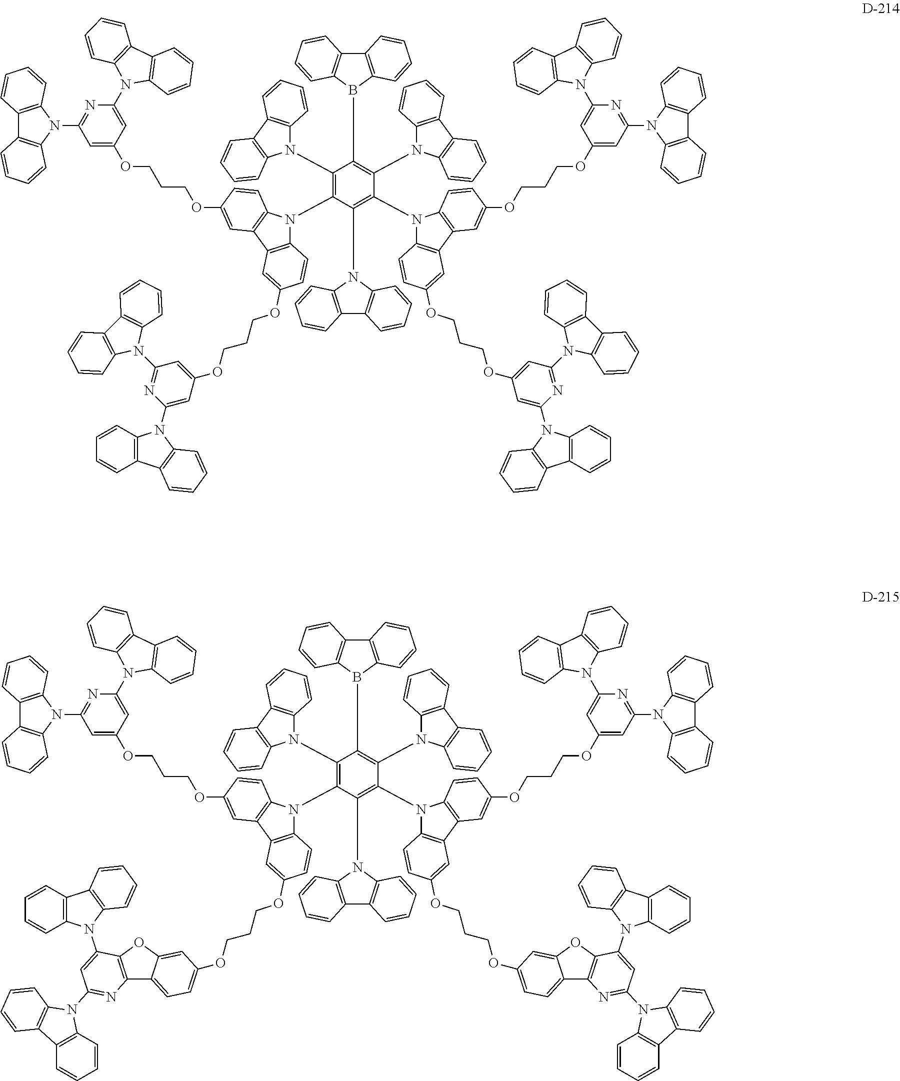

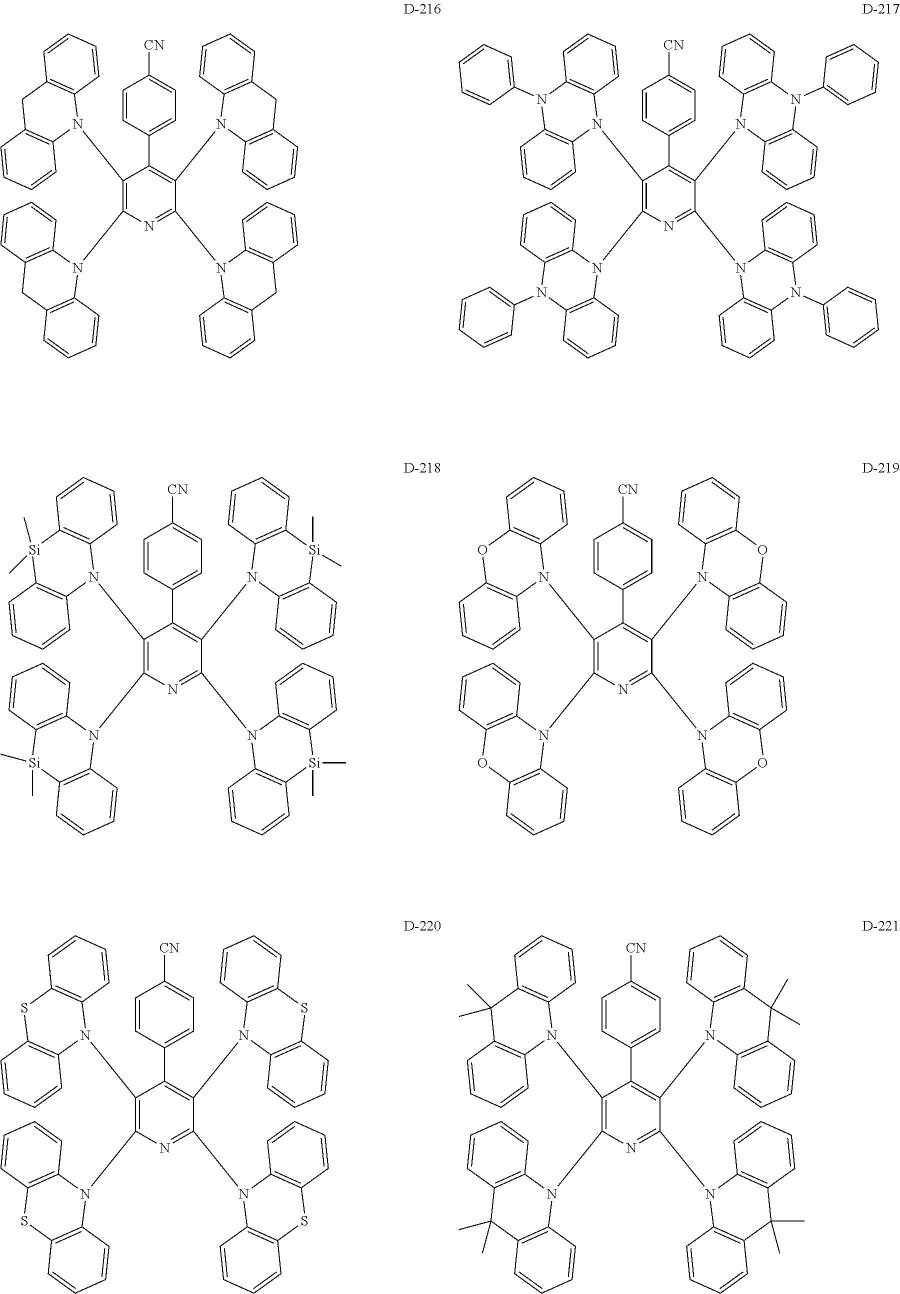

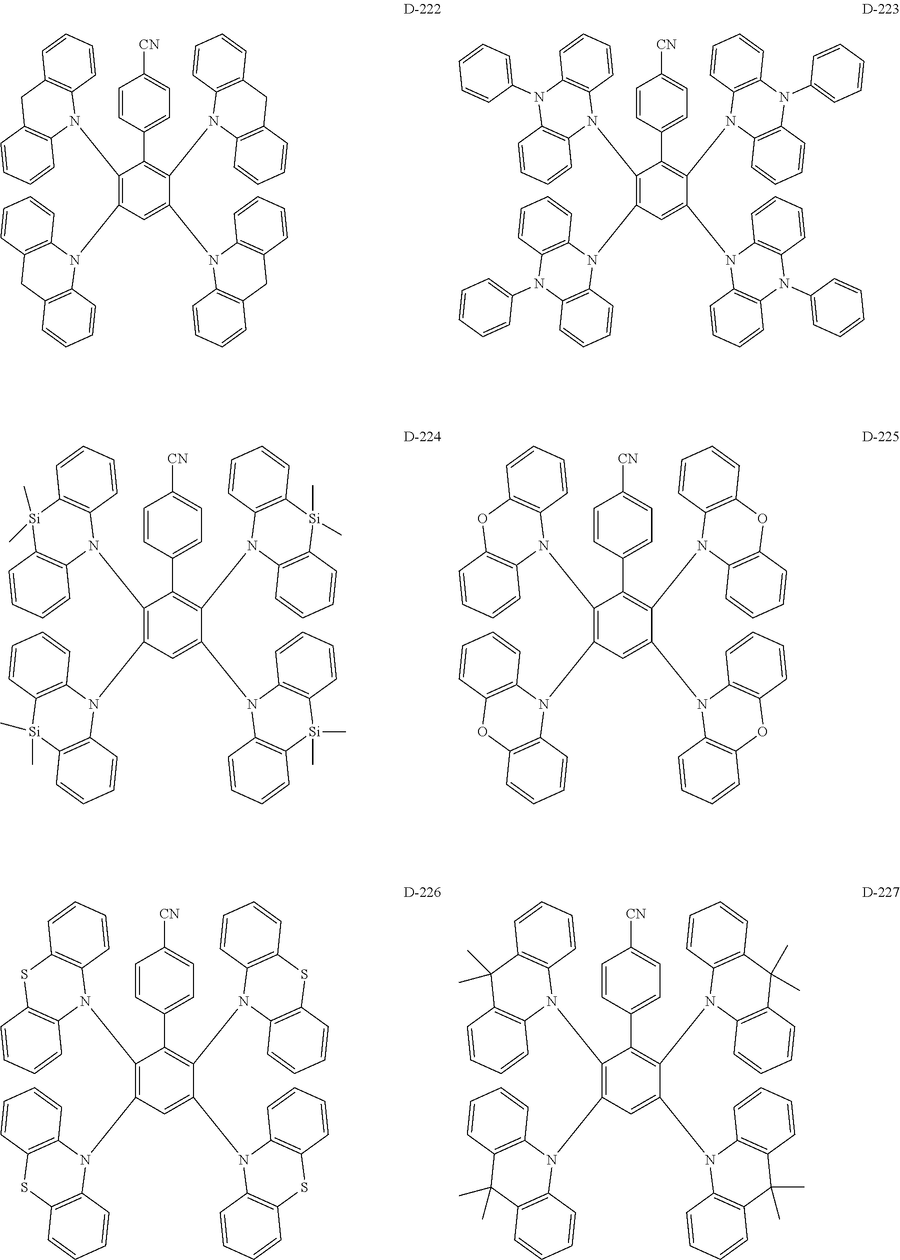

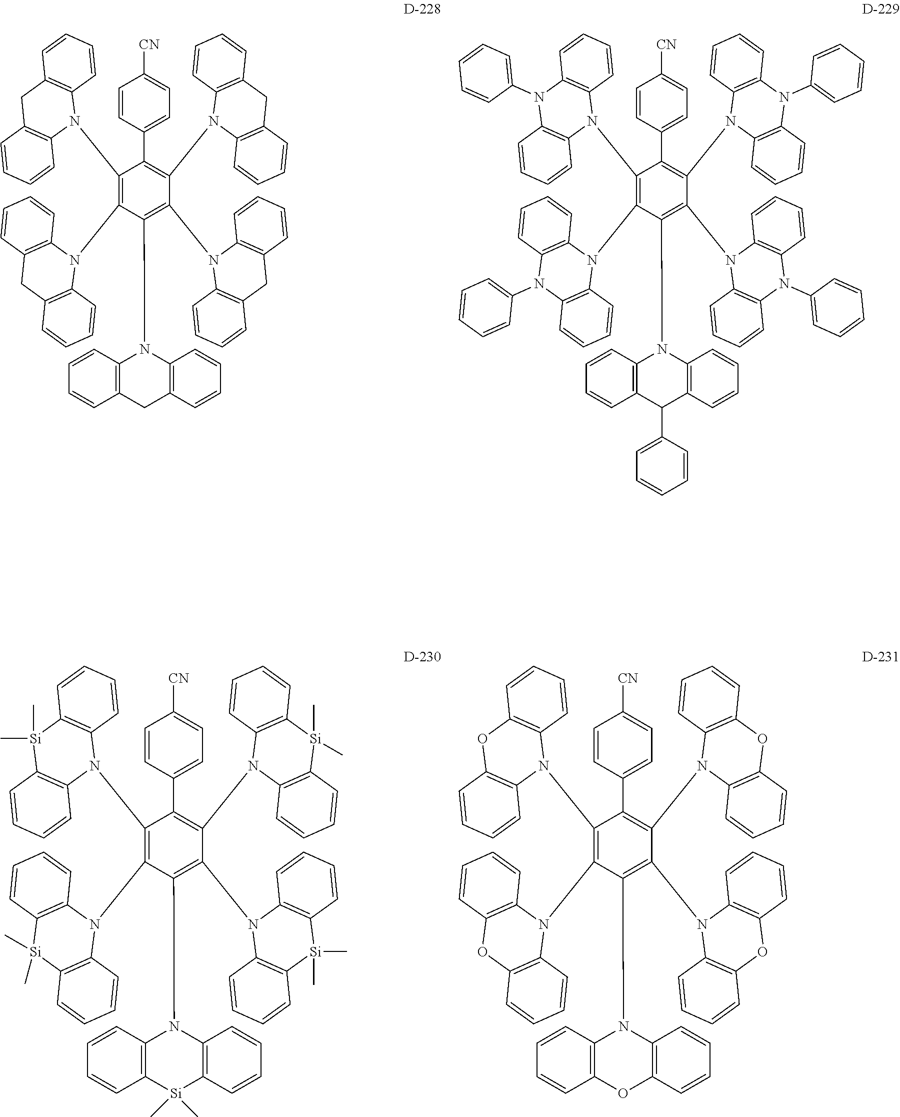

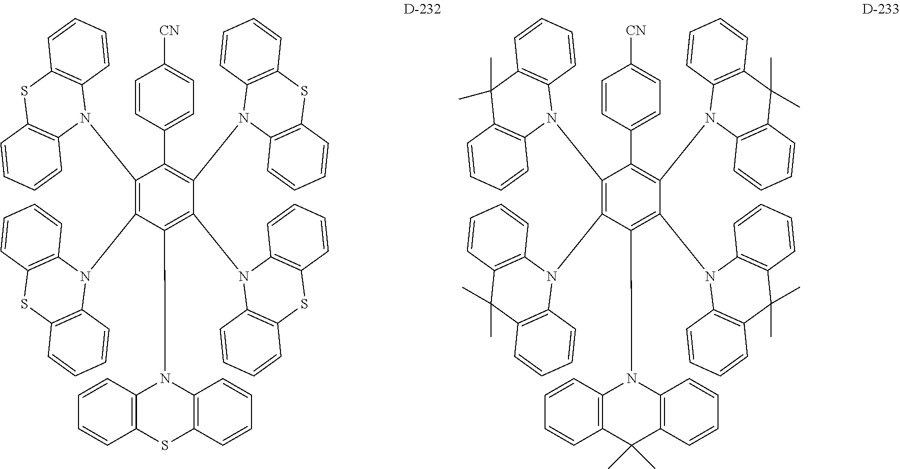

<Electron Density Distribution>

From the viewpoint of decreasing the ΔEst, it is preferable that the π-conjugated compound of the present invention has a HOMO and a LUMO that are substantially separated with each other in the molecule.

That is, the π-conjugated compound of the present invention preferably has a ΔEst, which is an absolute value of the energy difference between the lowest excited singlet level and the lowest excited triplet level, of 0.50 eV or less. This is because the originally forbidden intersystem crossing from lowest excited triplet energy level to the lowest excited singlet energy level can occur.

The distribution state of the HOMO and the LUMO may be obtained from the electron density distribution in the optimized structure by a molecular orbital calculation.

The structure optimization and the calculation of the electron density distribution of the π-conjugated compound of the present invention with a molecular orbital calculation may be done by employing a software of a molecular orbital calculation using B3LYP as a functional and 6-31G(d) as a base function for a calculation method. There is no limitation to the software, the same results may be obtained with any software.

In the present invention, as a molecular orbital calculation software, it was used Gaussian 09 made by the US Gaussian Inc. (Revision C.01, by M. J. Frisch et al., Gaussian Inc, 2010).

Here, the condition of “a HOMO and a LUMO being substantially separated” indicates the state in which the center portion of the HOMO orbital distribution and the center portion of the LUMO orbital distribution calculated with the above-described molecular calculation method are separated. More preferably, the HOMO orbital distribution and the LUMO orbital distribution are substantially not superimposed.

The separation state of the electron density distribution of the HOMO and the LUMO may be determined by making calculation of excited states with a Time-dependent DFT method starting from the optimized structure calculation using B3LYP as a functional and 6-31G(d) as a base function as described above. The excited state energy levels of S₁ and T₁ are obtained, and ΔEst is calculated from the scheme of: ΔEst=|E(S₁)−E(T₁)|. The smaller the calculated ΔEst, it indicates that the HOMO and the LUMO are more separated. In the present invention, an absolute value of ΔEst obtained by the above-described calculation method is preferably 0.5 eV or less, more preferably it is 0.2 eV or less.

<Lowest Excited Singlet Energy Level S₁>

In the present invention, the lowest excited singlet energy S₁ of the π-conjugated compound of the present invention may be determined by a common technique. Specifically, a target compound is deposited onto a quartz substrate to prepare a sample, and an absorption spectrum of the sample is measured at ambient temperature (300 K) (vertical axis: absorbance, horizontal axis: wavelength). A tangential line is drawn at the rising point of the absorption spectrum on the longer wavelength side, and the lowest excited singlet energy is calculated by a specific conversion expression on the basis of the wavelength at the point of intersection of the tangential line with the horizontal axis.

When the π-conjugated compound used in the present invention has a high aggregation property as a molecule itself, it is likely to cause molecular aggregation, and thus a thin film prepared from the compound may cause a measurement error due to molecular aggregation. In the present invention, the lowest excited singlet energy level S₁ is determined from, as an approximation, the peak wavelength of emission of a solution of the π-conjugated compound at room temperature (about 25° C.) in consideration of a relatively small Stokes shift of the π-conjugated compound and a very small structural change of the compound between the excited state and the ground state.

This determination process may use a solvent which does not affect the molecular aggregation state of the π-conjugated compound; for example, a non-polar solvent having a small solvent effect, such as cyclohexane or toluene.

<Lowest Excited Triplet Energy Level T₁>

In the present invention, the lowest excited triplet energy level (T₁) of the π-conjugated compound is determined on the basis of the photoluminescent (PL) properties of a solution or thin film of the compound.

For example, a thin film is prepared from a dilute dispersion of the π-conjugated compound, and the transient PL properties of the thin film are determined with a streak camera for separation of a fluorescent component and a phosphorescent component to determine the absolute value of the energy difference ΔEst therebetween. The lowest excited triplet energy level may be obtained from the lowest excited singlet energy level.

For measurement and evaluation, the absolute PL quantum yield was determined with an absolute PL Quantum yield measuring apparatus C9920-02 (manufactured by Hamamatsu Photonics K.K.). The emission lifetime was determined with a streak camera C4334 (manufactured by Hamamatsu Photonics K.K.) under excitation of the sample with a laser beam.

[Method for Producing π-Corrugated Compound]

In the method for producing the π-conjugated compound of the present invention, the substituents X₁ to X₅ are introduced to the benzene ring by a nucleophilic substitution reaction, respectively.

Specifically, 2,3,4,5,6-pentafluorobenzonitrile is dissolved in a solvent (THF, DMF, or NMP) and the benzonitrile derivative can be produced by reacting a carbazole compound or an azacarbazole compound which may have a substituent in the presence of a strong base (potassium carbonate, cesium carbonate, sodium hydride, or potassium hydride).

Before describing the organic EL element of the present invention, a light-emitting mode and a light-emitting material of the organic EL related to the technical idea will be described.

<Light Emission Mode of Organic EL>

As a light-emitting node of an organic EL, there are two types. One is “a phosphorescent emission type” which emits light when an excited triplet state returns to a ground state, and another one is “a fluorescent emission type” which emits light when an excited singlet state returns to a ground state.

When excitation is done by an electric field such as in the case of an organic EL element, a triplet exciton is produced with a probability of 75%, and a singlet exciton is produced with a probability of 25%. Consequently, it is possible that a phosphorescent emission has higher emission efficiency than fluorescent emission. The phosphorescent emission is an excellent mode to realize low electric consumption.

On the other hand, with respect to the fluorescent emission, it was found a method of using a TTA mechanism in which singlet excitons are generated from two triplet excitons (it is called as Triplet-Triplet Annihilation (TTA), or Triplet-Triplet Fusion (TTF)) to improve the emission efficiency.

In recent years, the group of Adachi found the following phenomenon. By achieving a small energy gap between the excited singlet state and the excited triplet state, it is allowed to occur a reverse intersystem crossing from the triplet state of lower energy level to the singlet state. This may be done by the Joule heat produced during the emission and/or the environmental temperature in which the light emission element is placed. As a result, it may be achieved a fluorescent emission in a yield of nearly 100% (it is called as a thermally activated delayed fluorescence: TADF). And it was found a compound enabling to occur this phenomenon (see Non-patent Document 1, for example).

<Phosphorescent Emission Material>

As described above, although the phosphorescent emission has theoretically an advantage of 3 times of the fluorescent emission, an energy deactivation (=phosphorescent emission) from the excited triplet state to the singlet ground state is a forbidden transition. In the same manner, the intersystem crossing from the excited singlet state to the excited triplet state is also a forbidden transition. Consequently, its rate constant is usually small. That is, since the transition takes place hardly, the lifetime of the exciton becomes long such as an order of millisecond or second. As a result, it is difficult to obtain a required emission.

However, when an emission occurs from a complex including a heavy atom of iridium or platinum, the rate constant of the above-described forbidden transition becomes larger by 3 orders due to the heavy metal effect of the center metal. It is possible to obtain a phosphorescence quantum efficiency of 100% when selection of the ligand is properly done.

However, in order to obtain an ideal emission, it is required to use a rare metal such as iridium or palladium, or a noble metal such as platinum. If a large amount of these metals are used, the reserves and the price of these metal will become problem.

<Fluorescent Emission Material>

A common fluorescent emission material is not required to be a heavy metal complex as in the case of a phosphorescent emission material. It may be applied a so-called organic compound composed of a combination of common elements such as carbon, oxygen, nitrogen and hydrogen. Further, non-metallic elements such as phosphor, sulfur, and silicon may be used. And a complex of typical element such as aluminum or zinc may be used. The variation of the materials is almost without limitation.

However, the conventional fluorescent emission material will use only 25% of the excitons to light emission. Therefore, it cannot be expected high emission efficiency as achieved by phosphorescent emission.

<Delayed Fluorescent Material>

[Excited triplet-triplet annihilation (TTA) delayed fluorescent material] A light emission mode employing a delayed fluorescence appeared to solve the problem of the fluorescent material. The TTA mode originated from the collision of the compounds at a triplet state may be described in the following Scheme. That is, in the past, a part of the triplet exciton is only convened to heat. This energy of the exciton is changed to a singlet exciton via an intersystem crossing to result in contributing to the light emission. In a practical organic EL element, it was proved that external quantum efficiency was double of the conventional fluorescent element.

Scheme: T*+T*→S*+S

(In the scheme, T* represents a triplet exciton, S* represents a singlet exciton, and S represents a ground state molecule.)

However, as can be seen from the above-described Scheme, only one singlet excitor is generated from two triplet excitors. Consequently, theoretically, 100% internal quantum efficiency cannot be obtained based on this mode.

<Thermally Activated Delayed Fluorescent (TADF) Compound>

A TADF mode, which is another type of high efficient fluorescent emission, is a mode enabling to resolve the problem.

A fluorescent material has an advantage of being molecular-designed without limitation as described above. Among the molecular-designed compounds, there are specific compounds having an energy level difference between an excited triplet state and an excited singlet state being in very close vicinity.

In spite of that fact that these compounds don't contain a heavy metal atom in the molecule, there occurs a reverse intersystem crossing reaction from the excited triplet state to the excited singlet state due to the small ΔEst value. This reaction will not usually occur. Further, since the rate constant of the deactivation from the excited singlet state to the ground state (=fluorescent emission) is extremely high, the triplet state will likely return to the ground state via the singlet state while emitting fluorescence, instead of thermally deactivating (non-radiative deactivation) to the ground state. As a result, in TADF mechanism, ideally, it is possible to realize fluorescent emission of 100%.

<Molecular Designing Idea Concerning ΔEst>

A molecular designing idea to reduce the ΔEst will be described.

In order to reduce the value of ΔEst, theoretically the most effective way is to minimize the spatial overlaps of the highest occupied molecular orbital (HOMO) and the lowest unoccupied molecular orbital (LUMO).

Generally, in the electronic orbitals of the molecule, it is known that HOMO has a distribution to an electron donating position and LUMO has a distribution to an electron withdrawing position. By introducing an electron donating structure and an electron withdrawing structure in the molecule, it is possible to keep apart the positions in which HOMO and LUMO exist.

For example, “Organic Photo-electronics in the commercialization stage” in Applied Physics vol. 82, no. 6, 2013 discloses the following. By introducing an electron withdrawing structure such as a cyano group, a sulfonyl group or a triazine group, and an electron donating structure such as a carbazole group or a diphenyl amino group, LUMO and HOMO are respectively made localized.

In addition, it is also effective to minimize the molecular structure change between the ground state and the excited triplet state of the molecule. As a means to minimize the structure change, it can cite a compound having an inflexible structure. Here, inflexibility indicates the state in which freely movable portions in the molecule are not abundant caused by preventing a free rotation of the bond between the rings in the molecule, or by introducing a condensed ring having a large π-conjugate plane, for example. In particular, by making the portion participating in the light emission to be rigid, it is possible to minimize, the molecular structure change in the excited state.

<Common Problem Possessed by TADF Compound>

A TADF compound possesses a variety of problems arisen from the aspects of the light emission mechanism and the molecular structure. A part of common problems possessed by a TADF compound will be described in the following.

In a TADF compound, it is required to keep apart the portions in which HOMO and LUMO exist as much as possible in order to minimize ΔEst. For this reason, the electronic state of the molecule becomes almost near the intramolecular CT state of a donor/accepter type (intramolecular charge transfer state).

When a plurality of these molecules exist, these molecules will be stabilized by making in proximity the donor portion in one molecule and the acceptor portion in other molecule. This stabilized condition is formed not only with 2 molecules, but it may be formed with 3 or 5 molecules. Consequently, there are produced a variety of stabilized conditions having a broad distribution. The shape of absorption spectrum or the emission spectrum will be broad. Further, even if a multiple molecular aggregation of 2 or more molecules does not formed, there may be formed a variety of existing conditions having different interaction directions or angles of two molecules. As a result, basically, the shape of absorption spectrum or the emission spectrum will be broad.

When the emission spectrum becomes broad, it will generate two major problems.

One is a problem of decreasing the color purity of the emission color. This is not so important when it is applied to an illumination use. However, when it is used for an electronic device, the color reproduction region becomes small. And the color reproduction of pure colors will become decreased. As a result, it is difficult to apply to a commercial product.

Another problem is the shortened wavelength of the rising wavelength in the short wavelength side of the emission spectrum (it is called as “fluorescent zero-zero band”). That is, the S₁ level becomes high (becoming higher energy level of the excited singlet energy).

When the fluorescent zero-zero band becomes shortened, the phosphorescent zero-zero band derived from T₁ (being lower than S₁) will become shortened (becoming higher T₁). Therefore, the host compound is required to have high S₁ and high T₁ in order to prevent the reverse energy transfer from the dopant.

This is a major problem. A host compound basically made of an organic compound will take plural and unstable chemical species conditions such as a cationic radical state, an anionic radical state and an excited state in an organic EL element. These chemical species may be made existed in relatively stable condition by expanding a π-conjugate system in the molecule.

However, in order to achieve high S₁ and high T₁, it is necessary to reduce or cut off the π-conjugated system in the molecule, which makes it difficult to achieve balance with stability. As a result, the life of the light-emitting element will be shorten.

Further, in a TADF compound without containing a heavy metal, the transition from the excited triplet state to the ground state is forbidden transition. The existing time at the excited triplet state (exciton lifetime) is extremely long such as in an order of several hundred microsecond to millisecond. Therefore, even if the T₁ energy level of the host compound is higher than that of the light-emitting material, it will be increased the probability of taking place a reverse energy transfer from the excited triplet state of the light-emitting material to the host compound due to the long lifetime. As a result, it is difficult to sufficiently make occur a required reverse intersystem crossing from the excited triplet state to the excited singlet state of the TADF compound. Instead, there occurs an unrequired reverse energy transfer to the host compound as a major route to result in failure to obtain insufficient emission efficiency.

In order to solve the above-described problem, it is required to make sharp a shape of an emission spectrum of the TADF compound, and to decrease the difference between the emission maximum wavelength and the rise of the emission spectrum. This may be achieved basically by reducing the change of the molecular structure of the excited singlet state and the excited triplet state.

Further, in order to prevent the reverse energy transfer to the host compound, it is effective to shorten the existing time of the excited triplet state of the TADF compound (exciton lifetime). In order to realize this, the possible ways to solve the problem are: to minimize the molecular structure change between the ground state and the excited triplet state; and to introduce a suitable substituent or an element to loosen the forbidden transition.

[Organic EL Element]

The organic EL element of the present invention is an organic electroluminescent element having at least a pair of electrodes and one or a plurality of light-emitting layers, and at least one of the light-emitting layers contains the π-conjugated compound.

Representative element constitutions used for an organic EL element of the present invention are as follows, however, the present invention is not limited to these.
(i) Anode/ light-emitting layer/ cathode
(ii) Anode/ light-emitting layer/ electron transport layer/ cathode
(iii) Anode/ hole transport layer/ light-emitting layer/ cathode
(iv) Anode/ hole transport layer/ light-emitting layer/ electron transport layer/ cathode
(v) Anode/ hole transport layer/ light-emitting layer/ electron transport layer/ electron injection layer/ cathode
(vi) Anode/ hole injection layer/ hole transport layer/ light-emitting layer/ electron transport layer/ cathode
(vii) Anode/ hole injection layer/ hole transport layer/ (electron blocking layer/) light emitting layer/ (hole blocking layer/) electron transport layer/ electron injection layer/ cathode

However, the present invention is not limited to this.

The light-emitting layer of the present invention is composed of one or a plurality of layers. When a plurality of layers are employed, it may be placed a non-light-emitting intermediate layer between the light-emitting layers. According to necessity, it may be provided with a hole blocking layer (it is also called as a hole barrier layer) or an electron injection layer (it is also called as a cathode buffer layer) between the light-emitting layer and the cathode. Further, it may be provided with an electron blocking layer (it is also called as an electron barrier layer) or an hole injection layer (it is also called as an anode buffer layer) between the light-emitting layer and the anode.

An electron transport layer according to the present invention is a layer having a function of transporting an electron. An electron transport layer includes an electron injection layer, and a hole blocking layer in a broad sense. Further, the organic EL element may include a plurality of electron transport layers.

A hole transport layer according to the present invention is a layer having a function of transporting a hole. A hole transport layer includes a hole injection layer, and an electron blocking layer in a broad sense. Further, the organic EL element may include a plurality of hole transport layers.

In the above-described typical element configuration, a layer excluding an anode and a cathode is also referred to as an “organic layer”.

(Tandem Structure)

An organic EL element of the present invention may be so-called a tandem structure element in which plural light-emitting units each containing at least one light-emitting layer arc laminated.

A representative example of an element constitution having a tandem structure is as follows.

Anode/ first light-emitting unit/ second light-emitting unit/ third light-emitting unit/ cathode; and

Anode/ first light-emitting unit/ intermediate layer/ second light-emitting unit/ intermediate layer/ third light-emitting unit/ cathode.

Here, the above-described first light-emitting unit, second light-emitting unit, and third light-emitting unit may be the same or different. It may be possible that two light-emitting units are the same and tile remaining one light-emitting unit is different.

In addition, the third light-emitting unit may not be provided. Otherwise, a further light-emitting unit or a further intermediate layer may be provided between the third light-emitting unit and the electrode.

The plural light-emitting units each may be laminated directly or they may be laminated through an intermediate layer. Examples of an intermediate layer are: an intermediate electrode, an intermediate conductive layer, a charge generating layer, an electron extraction layer, a connecting layer, and an intermediate insulating layer. Known composing materials may be used as long as it can form a layer which has a function of supplying an electron to an adjacent layer to the anode, and a hole to an adjacent layer to the cathode.

Examples of a material used in an intermediate layer are: conductive inorganic compounds such as ITO (indium tin oxide), IZO (indium zinc oxide), ZnO₂, TiN, ZrN, HfN, TiOX, VOX, CuI, InN, GaN, CuAlO₂, CuGaO₂, SrCu₂O₂, LaB₆, RuO₂, and Al; a two-layer film such as Au/Bi₂O₃; a multi-layer film such as SnO₂/Ag/SnO₂, ZnO/Ag/ZnO, Bi2O3/Au/Bi₂O₃, TiO₂/TiN/TiO₂, and TiO₂/ZrN/TiO₂; fullerene such as C₆₀; and a conductive organic layer such as oligothiophene, metal phthalocyanine, metal-free phthalocyanine, metal porphyrin, and metal-free porphyrin. However, the present invention is not limited to them.

Examples of a preferable constitution in the light-emitting unit are the constitutions of the above-described (i) to (vii) from which an anode and a cathode are removed. However, the present invention is not limited to them.

Examples of a tandem type organic EL element are described in: U.S. Pat. Nos. 6,337,492, 7,420,203, 7,473,923, 6,872,472, 6,107,734, 6,337,492, WO 2005/009087, JP-A 2006-228712, JP-A 2006-24791, JP-A 2006-49393, JP-A 2006-49394, JP-A 2006-49396, JP-A 2011-96679, JP-A 2005-340187, JP Patent 4711424, JP Patent 3496681, JP Patent 3884564, JP Patent 4213169, JP-A 2010-192719, JP-A 2009-076929. JP-A 2008-078414, JP-A 2007-059848, JP-A 2003-272860, JP-A 2003-045676. and WO 2005/094130. The constitutions of the elements and the composing materials are described in these documents, however, the present invention is not limited to them.

Each layer that constitutes an organic EL element of the present will be described in the following.

<<Light-Emitting Layer>>

A light-emitting layer used in the present invention is a layer which provide a place of emitting light via an exciton produce by recombination of electrons and holes injected from an electrode or an adjacent layer.

The light-emitting portion may be either within the light-emitting layer or at an interface between the light-emitting layer and an adjacent layer thereof. The total thickness of the light-emitting layer is not particularly limited, but from the viewpoint of achieving homogeneity of the film to be formed and preventing application of unnecessary high voltage at the time of light emission and achieving improvement of stability of luminescent color with respect to driving current, it is preferable to adjust in the range of 2 nm to 5 μm, more preferably in the range of 2 to 500 nm, and further preferably in the range of 5 to 200 nm.

In the present invention, the thickness of each light-emitting layer is preferably adjusted in the range of 2 nm to 1 μm, more preferably adjusted in the range of 2 to 200 nm, further preferably adjusted in the range of 3 to 150 nm.

The light-emitting layer preferably contains a light-emitting dopant (a light-emitting dopant compound, a dopant compound, also simply referred to as a dopant) and a host compound (a matrix material, a light-emitting host compound, also simply referred to as a host).

(1) Light-Emitting Dopant

As a light-emitting dopant, it is preferable to employ a fluorescence emitting dopant (also referred to as a fluorescent dopant and a fluorescence emitting compound), a delayed fluorescent dopant, and a phosphorescence emitting dopant (also referred to as a phosphorescent dopant and a phosphorescent emitting compound). In the present invention, it is preferable that one of the light-emitting layers contains the π-conjugated compound of the present invention.

In the present invention, it is preferable that the light-emitting layer contains a light-emitting dopant in an amount of 5 to 100 mass %, more preferably in an amount of 10 to 30 mass %.

A concentration of a light-emitting dopant in a light-emitting layer may be arbitrarily decided based on the specific compound employed and the required conditions of the device. A concentration of a light-emitting compound may be uniform in a thickness direction of the light-emitting layer, or it may have any concentration distribution.

The light-emitting dopant used in the present invention may be used in combination of two or more kinds. It may be a combination of light-emitting dopants each having a different structure, a π-conjugated compound of the present invention, or a combination of a fluorescent light-emitting compound and a phosphorescent light-emitting compound. Any required emission color will be obtained by this.

The color of light emitted by an organic EL element according to the present invention is specified as follows. The values determined via Spectroradiometer CS-1000 (produced by Konica Minolta, Inc.) are applied to the CIE chromaticity coordinate described in FIG. 4.16 on page 108 of “New Edition Color Science Handbook” (edited by The Color Science Association of Japan, University of Tokyo Press, 1985), whereby the color is specified.

In the present invention, it is also preferable that one or a plurality of light-emitting layers contain a plurality of light-emitting dopants having different emission colors and exhibit white light emission.

The combination of the light-emitting dopants exhibiting white color is not particularly limited, and for example, a combination of blue and orange., and a combination of blue, green and red can be cited.

The white color in the organic EL element according to the present invention is not particularly limited, and it may be a white color closer to orange or a white color closer to blue. It is preferable that “white” in the organic EL element of the present invention exhibits chromaticity in the CIE 1931 Color Specification System at 1000 cd/m2 in the region of x=0.39±0.09 and y=0.38±0.08, when measurement is done to 2-degree viewing angle front luminance via the aforesaid method.

(1.1) Phosphorescence Emitting Dopant

A phosphorescence emitting dopant according to the present invention (hereafter, it may be called as “a phosphorescent dopant”) will be described.

The phosphorescent dopant according to the present invention is a compound which is observed emission from an excited triplet state thereof. Specifically, it is a compound which emits phosphorescence at room temperature (25° C.) and exhibits a phosphorescence quantum yield of at least 0.01 at 25° C. The phosphorescence quantum yield is preferably at least 0.1.

The phosphorescence quantum yield will be determined via a method described in page 398 of “Spectroscopy II of 4th Edition Lecture of Experimental Chemistry 7” (1992, published by Maruzen Co. Ltd.). The phosphorescence quantum yield in a solution will be determined using appropriate solvents. However, it is only necessary for the phosphorescent dopant of the present invention to exhibit the above phosphorescence quantum yield (0.01 or more) using any of the appropriate solvents.

Two kinds of principles regarding emission of a phosphorescent dopant are cited. One is an energy transfer-type, wherein carriers recombine on a host compound on which the carriers are transferred to produce an excited state of the host compound, and then via transfer of this energy to a phosphorescent dopant, emission from the phosphorescence emitting dopant is realized. The other is a carrier trap-type, wherein a phosphorescence emitting dopant serves as a carrier trap and then carriers recombine on the phosphorescent dopant to generate emission from the phosphorescent dopant. In each case, the excited state energy level of the phosphorescent dopant is required to be lower than that of the host compound.

A phosphorescent dopant may be suitably selected and employed front the known materials used for a light-emitting layer for an organic EL element.

Examples of a known phosphorescent dopant are compounds described in the following publications.

Nature 395, 151 (1998), Appl. Phys. Lett. 78, 1622 (2001), Adv. Mater. 19, 739 (2007), Chem. Mater. 17, 3532 (2005), Adv. Mater. 17, 1059 (2005), WO 2009/100991, WO 2008/101842, WO 2003/040257, US 2006/835469, US 2006/0202194, US 2007/0087321, US 2005/0244673, Inorg. Chem. 40, 1704 (2001), Chem. Mater. 16, 2480 (2004), Adv. Mater. 16, 2003 (2004), Angew. Chem. Int. Ed. 2006, 45, 7800, Appl. Phys. Lett. 86, 153505 (2005), Chem. Lett. 34, 592 (2005), Chem. Commun. 2906 (2005), Inorg. Chem. 42, 1248 (2003), WO 2009/050290, WO 2002/015645, WO 2009/1000673, US 2002/0034656, U.S. Pat. No. 7,332,232, US 2009/0108737, US 2009,10039776, U.S. Pat. Nos. 6,921,915, 6,687,266, US 2007/0190359, US 2006/0008670, US 2009/0165846, US 2008/0015355, U.S. Pat. Nos. 7,250,226, 7,396,598, US 2006/0263635, US 2003/0138657, US 2003/0152802, U.S. Pat. No. 7,090,928, Angew. Chem. Int. Ed. 47, 1 (2008), Chem. Mater. 18, 5119 (2006), Inorg. Chem. 46, 4308 (2007), Organometallics 23, 3745 (2004), Appl. Phys. Lett. 74, 1361 (1999), WO 2002/002714, WO 2006/009024, WO 2006/056418, WO 2005/019373, WO 2005/123873, WO 2005/123873, WO 2007/004380, WO 2006/082742, US 2006/0251923, US 2005/0260441, U.S. Pat. Nos. 7,393,599, 7,534,505, 7,445,855, US 2007/0190359, US 2008/0297033, U.S. Pat. No. 7,338,722, US 2002/0134984, and U.S. Pat. No. 7,279,704, US 2006/098120, US 2006/103874, WO 2005/076380, WO 2010/032663, WO 2008/140115, WO 2007/052431, WO 2011/134013, WO 2011/157339, WO 2010/086089, WO 2009/113646, WO 2012/020327, WO 2011/051404, WO 2011/004639, WO 2011/073149, JP-A 2012-069737, JP Application No. 2011-181303, JP-A 2009-114086, JP-A 2003-81988, JP-A 2002-302671 and JP-A 2002-363552.

Among them, preferable other phosphorescent dopants are organic metal complexes containing Ir as a center metal. More preferable are complexes containing at least one coordination mode selected from a metal-carbon bond, a metal-nitrogen bond, a metal-oxygen bond and a metal-sulfur bond.

(1.2) Fluorescence Emitting Dopant

The fluorescence emitting dopant (hereinafter, it may be called as “fluorescent dopant”) will be described.

The fluorescent dopant according to the present invention is a compound capable of emitting light from an excited singlet state, and is not particularly limited as long as light emission from an excited singlet state is observed.

As the fluorescent dopant of the present invention, it may be used a π-conjugated compound of the preset invention. Otherwise, it may be suitably selected from the known fluorescent dopants and delayed fluorescent dopants used in a light emitting layer of an organic EL element.

Examples of the fluorescent dopant according to the present invention are: an anthracene derivative, a pyrene derivative, a chrysene derivative, a fluoranthene derivative, a perylene derivative, a fluorene derivative, an arylacetylene derivative, a styrylarylene derivative, a styrylamine derivative, an arylamine derivative, a boron complex, a coumarin derivative, a pyran derivative, a cyanine derivative, a croconium derivative, a squarylium derivative, an oxobenzanthracene derivative, a fluorescein derivative, a rhodamine derivative, a pyrylium derivative, a perylene derivative, a polythiophene derivative, and a rare earth complex compound.

Specific examples of the delayed fluorescent dopant are compounds described in: WO 2011/156793, JP-A 2011-213643, and JP-A 2010-93181. However, the present invention is not limited to them.

(2) Host Compound

A host compound according to the present invention is a compound which mainly plays a role of injecting or transporting a charge in a light-emitting layer. In an organic EL element, an emission from the host compound itself is substantially not observed.

Preferably, it is a compound exhibiting a phosphorescent emission yield of less than 0.1 at room temperature (25° C.), more preferably a compound exhibiting a phosphorescent emission yield of less than 0.01. Among the compounds incorporated in the light-emitting layer, a mass ratio of the host compound in the light-emitting layer is preferably at least 20%.

It is preferable that the excited energy level of the host compound than the excited energy level of the dopant contained in the same layer.

The host compounds may be used singly or may be used in combination of two or more compounds. By using a plurality of the other host compounds, it is possible to adjust transfer of charge, thereby it is possible to achieve an organic EL element of high efficiency.

The host compound is not specifically limited. The π-conjugated compound of the present invention may be used. A known compound previously used in an organic EL element may be used. It may be a compound having a low molecular weight, or a polymer having a high molecular weight. Further, it may be a compound having a reactive group such as a vinyl group or an epoxy group.

From the viewpoint of reverse energy transfer, those having an excited energy level higher than the excited singlet energy level of the dopant are preferable, and those having an excited triplet energy level higher than the excited triplet energy level of the dopant are more preferable.

A host compound bears the function of transfer of the carrier and generation of an exciton in the light-emitting layer. Therefore, it is preferable that the host compound will exist as a stable state in all of the active species of a cation radical state, an anion radial state and an excited state, and that it will not make chemical reactions such as decomposition and addition. Further, it is preferable that the host molecule will not move in the layer with an Angstrom level when an electric current is applied.

In addition, in particular, when the light-emitting dopant to be used in combination is a compound exhibiting TADF emission, since the lifetime of the excited triplet state of the TADF compound is long, it is required an appropriate design of a molecular structure to prevent the host compound from having a lower T1 level such as: the host compound has a high T1 energy level; the host compounds will not form a low T1 state when aggregated each other; the TADF compound and the host compound will not form an exciplex; and the host compound will not form an electromer by applying an electric field.

In order to satisfy the above-described requirements, it is required that: the host compound itself has a high hopping mobility; the host compound has high hole hopping mobility; and the host compound has small structural change when it becomes an excited triplet state. As a representative host compound satisfying these requirements, preferable compounds are: a compound having a high T1 energy such as a carbazole structure, an azacarbazole structure, a dibenzofuran structure, a dibenzothiophene structure and an azadibenzofuran structure.

Such a known host compound has a hole transporting ability or an electron transporting ability and prevents elongation of an emission wavelength. In addition, from the viewpoint of stably driving an organic EL element at high temperature, it is preferable that a host compound has a high glass transition temperature (Tg) of 90° C. or more. In addition, front the viewpoint of stably driving an organic EL element at high temperature, it is preferable that a host compound has a high glass transition temperature (Tg) of 90° C. or more, more preferably, has a Tg of 120° C. or more.

Here, a glass transition temperature (Tg) is a value obtained using DSC (Differential Scanning Calorimetry) based on the method in conformity to JIS-K-7121.

As specific examples of a known host compound used in an organic EL element of the present invention, the compounds described in the following Documents are cited. However, the present invention is not limited to them.

Japanese patent application publication (JP-A) Nos. 20010-257076, 2002-308855, 2001-313179, 2002-319491, 2001-357977, 2002-334786, 2002-8860, 2002-334787, 2002-15871, 2002-334788, 2002-43056, 2002-334789, 2002-75645, 2002-338579, 2002-105445, 2002-343568, 2002-141173, 2002-352957, 2002-203683, 2002-363227, 2002-231453, 2003-3165, 2002-234888, 2003-27048, 2002-255934, 2002-260861, 2002-280183, 2002-299060, 2002-302516, 2002-305083, 2002-305084 and 2002-308837; US Patent Application Publication (US) Nos. 2003/0175553, 2006/0280965, 2005/0112407, 2009/0017330, 2009/0030202, 2005/0238919; WO 2001/039234, WO 2009/021126, WO 2008/056746, WO 2004/093 207, WO 2005/089025, WO 2007/063796, WO 2007/063754, WO 2004/107822, WO 2005/030900, WO 2006/114966, WO 2009/086028, WO 2009/003898, WO 2012/023947, JP-A 2008-074939, JP-A 2007-254297, and EP 2034538.

<<Electron Transport Layer>>

In the present invention, the electron transport layer is made of a material having a function of transporting electrons and may have a function of transmitting electrons injected from the cathode to the light-emitting layer.

The total thickness of the electron transport layer in the present invention is not particularly limited, but is usually in the range of 2 nm to 5 μm, more preferably in the range of 2 to 500 nm, and further preferably in the range of 5 to 200 nm.

As a material used for an electron transport layer (hereinafter, it is called as “an electron transport material”), it is only required to have either a property of injection or transport of electrons, or a barrier to holes. The π-conjugated compound of the present invention may be used, and any of the conventionally known compounds may be selected and they may be employed.

Examples of the conventionally known compound include: a nitrogen-containing aromatic heterocyclic derivative (a carbazole derivative, an azacarbazole derivative (a compound in which one or more carbon atoms constituting the carbazole ring are substitute with nitrogen atoms), a pyridine derivative, a pyrimidine derivative, a pyrazine derivative, a pyridazine derivative, a triazine derivative, a quinoline derivative, a quinoxaline derivative, a phenanthroline derivative, an azatriphenylene derivative, an oxazole derivative, a thiazole derivative, an oxadiazole derivative, a thiadiazole derivative, a triazole derivative, a benzimidazole derivative, a benzoxazole derivative, and a benzothiazole derivative); a dibenzofuran derivative, a dibenzothiophene derivative, a silole derivative; and an aromatic hydrocarbon ring derivative (a naphthalene derivative, an anthracene derivative and a triphenylene derivative).

Further, metal complexes having a ligand of a 8-quinolinol structure or dibnenzoquinolinol structure such as tris(8-quinolinol)aluminum (Alq₃), tris(5,7-dichloro-8-quinolinol)aluminum, tris(5,7-dibromo-8-quinolinol)aluminum, tris(2-methyl-8-quinolinol)aluminum, tris(5-methyl-8-quinolinol)aluminum and bis(8-quinolinol)zinc (Znq); and metal complexes in which a central metal of the aforesaid metal complexes is substituted by In, Mg, Cu, Ca, Sn, Ga or Pb, may be also utilized as an electron transport material.

Further, a metal-free or metal phthalocyanine, or a compound whose terminal is substituted by an alkyl group or a sulfonic acid group, may be preferably utilized as an electron transport material. A distyrylpyrazine derivative, which is exemplified as a material for a light-emitting layer, may be used as an electron transport material. Further, in the same manner as used for a hole injection layer and a hole transport layer, an inorganic semiconductor such as an n-type Si and an n-type SiC may be also utilized as an electron transport material.

A polymer material which is introduced these compounds in the polymer side-chain or a polymer main chain may be used.

In an electron transport layer according to the present invention, it is possible to employ an electron transport layer of a higher n property (electron rich) which is doped with a dopant as a guest material. Such dopants includes n-type dopants such as metal compounds including metal complexes and metal halides. Examples of the electron transport layer having such a structure includes those described in JP-A-04-297076, JP-A-10-270172, JP-A-2000-196140, JP-A-2001-102175, J. Appl. Phys., 95, 5773 (2004) and the like.

Although the present invention is not limited thereto, preferable examples of a known electron transport material used in an organic EL element of the present invention are compounds described in the following publications.

U.S. Pat. Nos. 6,528,187, 7,230,107, US 2005/0025993, US 2004/0036077, US 2009/0115316, US 2009/0101870, US 2009/0179554, WO 2003/060956, WO 2008/132085, Appl. Phys. Lett. 75, 4 (1999), Appl. Phys. Lett. 79, 449 (2001), Appl. Phys. Lett. 81, 162 (2002), Appl. Phys. Lett. 81, 162 (2002), Appl. Phys. Lett. 79, 156 (2001), U.S. Pat. No. 7,964,293, US 2009/030202, WO 2004/080975, WO 2004/063159, WO 2005/085387, WO 2006/067931, WO 2007/086552, WO 2008/114690, WO 2009/069442, WO 2009/066779, WO 2009/054253, WO 2011/086935, WO 2010/150593, WO 2010/047707, EP 2311826, JP-A 2010-251675, JP-A 2009-209133, JP-A 2009-124114, JP-A 2008-277810, JP-A 2006-156445, JP-A 2005-340122, JP-A 2003-45662, JP-A 2003-31367, JP-A 2003-282270, and WO 2012/115034.

Examples of a preferable electron transport material in the present invention are: a pyridine derivative, a pyrimidine derivative, a pyrazine derivative, a triazine derivative, a dibenzofuran derivative, a dibenzothiophene derivative, a carbazole derivative, an azacarbazole derivative, and a benzimidazole derivative.

An electron transport material may be used singly, or may be used in combination of plural kinds of compounds.

<<Hole Blocking Layer>>

A hole blocking layer is a layer provided with a function of an electron transport layer in a broad meaning. Preferably, it contains a material having a function of transporting an electron, and having very small ability of transporting a hole. It will improve the recombination probability of an electron and a hole by blocking a hole while transporting an electron.

Further, a composition of an electron transport layer described above may be appropriately utilized as a hole blocking layer of the present invention when needed.

A hole blocking layer is preferably provided adjacent to the cathode side of the light-emitting layer

A thickness of a hole blocking layer is preferably in the range of 3 to 100 nm, and more preferably, it is in the range of 5 to 30 μm.

With respect to a material used for a hole blocking layer, the material used in the aforesaid electron transport layer including the π-conjugated compound of the present invention is suitably used. Further, the material used as the aforesaid host compound including the π-conjugated compound of the present invention is also suitably used for a hole blocking layer.

<<Electron Injection Layer>>

An electron injection layer (it is also called as “a cathode buffer layer”) according to the present invention is a layer which is arranged between a cathode and a light-emitting layer to decrease an operating voltage and to improve an emission luminance. An example of an electron injection layer is detailed in volume 2, chapter 2 “Electrode materials” (pp. 123-166) of “Organic EL Elements and Industrialization Front thereof (Nov. 30, 1998, published by N.T.S. Co. Ltd.)”.

In the present invention, an electron injection layer is provided according to necessity, and as described above, it is placed between a cathode and a light-emitting layer, or between a cathode and an electron transport layer.

An electron injection layer is preferably a very thin layer. The layer thickness thereof is preferably in the range of 0.1 to 5 nm depending on the materials used. Further, it may be a non-uniform film in which the constituent material is intermittently present.

An election injection layer is detailed in JP-A Nos. 6-325871, 9-17574, and 10-74586. Examples of a material preferably used in an election injection layer include: a metal such as strontium and aluminum; an alkaline metal compound such as lithium fluoride, sodium fluoride, or potassium fluoride, an alkaline earth metal compound such as magnesium fluoride; a metal oxide such as aluminum oxide; and a metal complex such as lithium 8-hydroxyquinolate (Liq). It is possible to use the aforesaid electron transport materials including the π-conjugated compound.

The material used in the above-described election injection layer may be used singly, or plural kinds may be used together.

<<Hole Transport Layer>>

In the present invention, a hole transport layer contains a material having a function of transporting a hole. A hole transport layer is only required to have a function of transporting a hole injected from an anode to a light-emitting layer.

The total layer thickness of a hole transport layer of the present invention is not specifically limited, however, it is generally in the range of 5 nm to 5 μm, preferably in the range of 2 to 500 nm, and more preferably in the range of 5 nm to 200 nm.

A material used in a hole transport layer (hereinafter, it is called as “a hole transport material”) is only required to have any one of properties of injecting or transporting a hole, and a barrier property to an electron. The π-conjugated compound of the present invention may be used, or any of conventionally known compounds may be selected and used.

Examples of the hole transport material include: a porphyrin derivative, a phthalocyanine derivative, an oxazole derivative, an oxadiazole derivative, a triazole derivative, an imidazole derivative, a pyrazoline derivative, a pyrazolone derivative, a phenylenediamine derivative, a hydrazone derivative, a stilbene derivative, a polyarylalkane derivative, a triarylamine derivative, a carbazole derivative, an indolocarbazole derivative, an isoindole derivative, an acene derivative of anthracene or naphthalene, a fluorene derivative, a fluorenone derivative, polyvinyl carbazole, a polymer or an oligomer containing an aromatic amine in a side chain or a main chain, polysilane, and a conductive polymer or an oligomer (e.g., PEDOT:PSS, an aniline type copolymer, polyaniline and polythiophene).

Examples of a triarylamine derivative include: a benzidine type represented by α-NPD, a star burst type represented by MTDATA, a compound having fluorenone or anthracene in a triarylamine bonding core.

A hexaazatriphenylene derivative described in JP-A Nos. 2003-519432 and 2006-135145 may be also used as a hole transport material.

In addition, it is possible to employ an electron transport layer of a higher p property which is doped with impurities. As its example, listed are those described in each of JP-A Nos. 4-297076, 2000-196140, and 2001-102175, as well as in J. Appl. Phys., 95, 5773 (2004).

Further, it is possible to employ so-called p-type hole transport materials, and inorganic compounds such as p-type Si and p-type SiC, as described in JP-A No. 11-251067, and J. Huang et at reference (Applied Physics Letters 80 (2002), p. 139). Moreover, an orthometal compounds having Ir or Pt as a center metal represented by Ir(ppy)₃ are also preferably used.

Although the above-described compounds may be used as a hole transport material, preferably used are: a triarylamine derivative, a carbazole derivative, an indolocarbazole derivative, an azatriphenylene derivative, an organic metal complex, a polymer or an oligomer incorporated an aromatic amine in a main chain or in a side chain.

Specific examples of a known hole transport material used in an organic EL element of the present invention are compounds in the aforesaid publications and in the following publications. However, the present invention is not limited to them.

Examples of the publication are: Appl. Phys. Lett. 69, 2160(1996), J. Lumin. 72-74, 985(1997), Appl. Phys. Lett. 78, 673(2001), Appl. Phys. Lett. 90, 183503(2007), Appl. Phys. Lett. 51, 913(1987), Synth. Met. 87, 171(1997), Synth. Met. 91, 209(1997), Synth. Met. 111, 421(2000), SID Symposium Digest, 37, 923(2006), J. Mater. Chem. 3, 319(1993), Adv. Mater. 6, 677(1994), Chem. Mater. 15, 3148(2003), US 2003/0162053, US 2002/0158242, US 2006/0240279, US 2008/0220265, U.S. Pat. No. 5,061,569, WO 2007/002683, WO 2009/018009, EP 650955, US 2008/0124572, US 2007/0278938, US 2008/0106190, US 2008/0018221, WO 2012/115034, JP-A 2003-519432, JP-A 2006-135145, and U.S. patent application Ser. No. 13/585,981.

A hole transport material may be used singly or may be used in combination of plural kinds of compounds.

<<Electron Blocking Layer>>

An electron blocking layer is a layer provided with a function of a hole transport layer in a broad meaning. Preferably, it contains a material having a function of transporting a hole, and having very small ability of transporting an electron. It will improve the recombination probability of an electron and a hole by blocking an electron while transporting a hole.

Further, a composition of a hole transport layer described above may be appropriately utilized as an electron blocking layer of an organic EL element when needed.

An electron blocking layer is preferably provided adjacent to the anode side of the light-emitting layer.

A thickness of an electron blocking layer is preferably in the range of 3 to 100 nm, and more preferably, it is in the range of 5 to 30 nm.

With respect to a material used for an electron blocking layer, the material used in the aforesaid hole transport layer including the π-conjugated compound of the present invention is suitably used, and further, the material used as the aforesaid host compound is also suitably used for an electron blocking layer.

<<Hole Injection Layer>>

A hole injection layer (it is also called as “an anode buffer layer”) is a layer which is arranged between an anode and a light-emitting layer to decrease an operating voltage and to improve an emission luminance. An example of a hole injection layer is detailed in volume 2, chapter 2 “Electrode materials” (pp. 123-166) of “Organic EL Elements and industrialization Front thereof (Nov. 30, 1998, published by N.T.S. Co. Ltd.)”.

A hole injection layer of the present invention is provided according to necessity, and as described above, it is placed between an anode and a light-emitting layer, or between an anode and a hole transport layer.

A hole injection layer is also detailed in JP-A Nos. 9-45479, 9-260062 and 8-288069. As materials used in the hole injection layer, it is cited the same materials used in the aforesaid hole transport layer including the π-conjugated compound of the present invention.

Among them, preferable materials are: a phthalocyanine derivative represented by copper phthalocyanine; a hexaazatriphenylene derivative described in JP-A Nos. 2003-519432 and 2006-135145; a metal oxide represented by vanadium oxide; a conductive polymer such as amorphous carbon, polyaniline (or called as emeraldine) and polythiophene; an orthometalated complex represented by tris(2-phenylpyridine) iridium complex; and a triarylamine derivative.

The materials used in the above-described hole injection layer may be used singly or may be used in combination of plural kinds of compounds.

<<Other Additive>>

The above-described organic layer of the present invention may further contain other additive.

Examples of the additive are: halogen elements such as bromine, iodine and chlorine, and a halide compound; and a compound, a complex and a salt of an alkali metal, an alkaline earth metal and a transition metal such as Pd, Ca and Na.

Although a content of the additive may be arbitrarily decided, preferably, it is 1,000 ppm or less based on the total mass of the layer containing the additive, more preferably, it is 500 ppm or less, and still more preferably, it is 50 ppm or less.

In order to improve a transporting property of an electron or a hole, or to facilitate energy transfer of an excitor), the content of the additive is not necessarily within these range.

<<Forming Method of Organic Layers>>

The method for forming an organic layer (a hole an injection layer, a hole transport layer, a light-emitting layer, a hole blocking layer, an electron transport layer, and an electron injection layer) in the present invention will be described.

Forming methods of organic layers according to the present invention are not specifically limited. They may be formed by using a known method such as a vacuum vapor deposition method and a wet method (wet process).

Examples of a wet process include: a spin coating method, a cast method, an inkjet method, a printing method, a die coating method, a blade coating method, a roll coating method, a spray coating method, a curtain coating method, and a LB method (Langmuir Blodgett method). From the viewpoint of getting a uniform thin layer with high productivity, preferable are method highly appropriate to a roll-to-roll method such as a die coating method, a roll coating method, an inkjet method, and a spray coating method.

Examples of a liquid medium to dissolve or to disperse a material for organic layers according to the present invention include: ketones such as methyl ethyl ketone and cyclohexanone; aliphatic esters such as ethyl acetate; halogenated hydrocarbons such as dichlorobenzene; aromatic hydrocarbons such as toluene, xylene, mesitylene, and cyclohexylbenzene, aliphatic hydrocarbons such as cyclohexane, decalin, and dodecane; organic solvents such as DMF and DMSO.

These will be dispersed with a dispersion method such as an ultrasonic dispersion method, a high shearing dispersion method and a media dispersion method.

A different film forming method may be applied to every organic layer. When a vapor deposition method is adopted for forming each layer, the vapor deposition conditions may be changed depending on the compounds used. Generally, the following ranges are suitably selected for the conditions, heating temperature of boat: 50 to 450° C., level of vacuum: 10⁻⁶ to 10⁻² Pa, vapor deposition rate: 0.01 to 50 nm/sec, temperature of substrate: −50 to 300° C., and layer thickness: 0.1 nm to 5 μm, preferably 5 to 200 nm.

Formation of each organic layer in the present invention is preferably continuously carried out from a hole injection layer to a cathode with one time vacuuming. It may be taken out on the way, and a different layer forming method may be employed. In that case, the operation is preferably done under a dry inert gas atmosphere.

<<Anode>>

As an anode of an organic EL element, a metal having a large work function (4 eV or more, preferably, 4.5 eV or more), an alloy, and a conductive compound and a mixture thereof are utilized as an electrode substance. Specific examples of the electrode substance are: metals such as Au; transparent conductive materials such as CuI, indium tin oxide (ITO), SnO₂, and ZnO. Further, a material such as IDIXO (In₂O₃—ZnO), which may form an amorphous and transparent electrode, may also be used.

As for an anode, these electrode substances may be made into a thin layer by a method such as a vapor deposition method or a sputtering method; followed by making a pattern of a desired form by a photolithography method. Otherwise, when the requirement of pattern precision is not so severe (about 100 μm or more), a pattern may be formed through a mask of a desired for at the time of layer formation with a vapor deposition method or a sputtering method using the above-described material.

Alternatively, when a coatable substance such as an organic conductive compound is employed, it is possible to employ a wet film forming method such as a printing method or a coating method. When emitted light is taken out from the anode, the transmittance is preferably set to be 10% or more. A sheet resistance of the anode is preferably a few hundred Ω/sq or less.

Further, although a layer thickness of the anode depends on a material, it is generally selected in the range of 10 nm to 1 μm, and preferably in the range of 10 to 200 nm.

<<Cathode>>

As a cathode, a metal having a small work function (5 eV or less) (it is called as an electron injective metal), an alloy, a conductive compound and a mixture thereof are utilized as an electrode substance. Specific examples of the aforesaid electrode substance includes: sodium, sodium-potassium alloy, magnesium, lithium, silver, magnesium/copper mixture, a magnesium/silver mixture, a magnesium/aluminum mixture, a magnesium/indium mixture, an aluminum/aluminum oxide (Al₂O₃) mixture, indium, a lithium/aluminum mixture, aluminum, and a rare earth metal.

Among them, with respect to an electron injection property and durability against oxidation, preferable are: a mixture of election injecting metal with a second metal which is stable metal having a work function larger than the electron injecting metal. Examples thereof are: a magnesium/silver mixture, a magnesium/aluminum mixture, a magnesium/indium mixture, an aluminum/aluminum oxide (Al₂O₃) mixture, a lithium/aluminum mixture and aluminum.

A cathode may be made by using these electrode substances with a method such as a vapor deposition method or a sputtering method to form a thin film. Alternatively, when a coatable substance such as metal nanoparticles is employed, it is possible to employ a wet film forming method such as a printing method or a coating method. The sheet resistance of the cathode is preferably a few hundred Ω/sq or less. A layer thickness of the cathode is generally selected in the range of 10 nm to 5 μm, and preferably in the range of 50 to 200 nm.

In order to transmit emitted light, it is preferable that one of an anode and a cathode of an organic EL element is transparent or translucent for achieving an improved luminescence.

Further, after forming a layer of the aforesaid metal having a thickness of 1 to 20 nm on the cathode, it is possible to prepare a transparent or translucent cathode by providing with a conductive transparent material described in the description for the anode thereon. By applying this process, it is possible to produce an element in which both an anode and a cathode are transparent.

<<Support Substrate>>

A support substrate which may be used for an organic EL element of the present invention is not specifically limited with respect to types such as glass and plastics. Hereinafter, the support substrate may be also called as substrate body, substrate, base material, or support. They may be transparent or opaque. However, a transparent support substrate is preferable when the emitting light is taken from the side of the support substrate. Support substrates preferably utilized includes such as glass, quartz and transparent resin film. A specifically preferable support substrate is a resin film capable of providing an organic EL element with a flexible property.

Examples of the resin film include: polyesters such as polyethylene terephthalate (PET) and polyethylene naphthalate (PEN), polyethylene, polypropylene, cellophane, cellulose esters and their derivatives such as cellulose diacetate, cellulose triacetate (TAC), cellulose acetate butyrate, cellulose acetate propionate (CAP), cellulose acetate phthalate, and cellulose nitrate, polyvinylidene chloride, polyvinyl alcohol, polyethylene vinyl alcohol, syndiotactic polystyrene, polycarbonate, norbornene resin, polymethyl pentene, polyether ketone, polyimide, polyether sulfone (PES), polyphenylene sulfide, polysulfones, polyether imide, polyether ketone imide, polyamide, fluororesin, Nylon, polymethyl methacrylate, acrylic resin, polyallylates and cycloolefin resins such as ARTON (trademark, made by JSR Co. Ltd.) and APEL (trademark, made by Mitsui Chemicals, Inc.).

On a surface of a resin film, it may be formed a film incorporating an inorganic or an organic compound or a hybrid film incorporating both compounds. It is preferable that the film is a barrier film having a water vapor permeability of 0.01 g/(m²·24 h) or less (25±0.5° C., relative humidity of (90±2)%) determined by the method based on JIS K 7129-1992. It is more preferable that the film is a high barrier film having an oxygen permeability of 10⁻³ mL(m²·24 h·atm) or less determined by the method based on JIS K 7126-1987, and a water vapor permeability of 10⁻⁵ mL(m²·24 h) or less.

The material for forming the gas barrier film may be any material that has a function of suppressing infiltration of a material that causes deterioration of the element such as water and oxygen. For example, it is possible to employ silicon oxide, silicon dioxide, and silicon nitride. Further, in order to improve the brittleness of the aforesaid film, it is more preferable to achieve a laminated layer structure of inorganic layers and organic layers. The laminating order of the inorganic layer and the organic layer is not particularly limited, but it is preferable that both are alternatively laminated a plurality of times.

There is no particular limitation on the method of forming the gas barrier film. Examples of an employable method include a vacuum deposition method, a sputtering method, a reactive sputtering method, a molecular beam epitaxy method, a cluster ion beam method, an ion plating method, a plasma polymerization method, a plasma CVD method, a laser CVD method, a thermal CVD method, and a coating method. Of these, specifically preferred is a method employing an atmospheric pressure plasma polymerization method, described in JP-A 2004-68143.

Examples of the opaque support substrate include metal plates such aluminum or stainless steel films, opaque resin substrates, and ceramic substrates.

An external extraction quantum efficiency of light emitted by the organic EL element of the present invention is preferably 1% or more at room temperature, but is more preferably 5% or more.

External extraction quantum efficiency (%)=(Number of photons emitted by the organic EL element to the exterior/Number of electrons fed to organic EL element)×100.

Further, it may be used simultaneously a color hue improving filter such as a color filter, or it may be used simultaneously a color conversion filter which convert emitted light color from the organic EL element to multicolor by employing fluorescent materials.

<Production Method of organic EL Element>

The method for forming an organic layer (a hole an injection layer, a hole transport layer, a light-emitting layer, a hole blocking layer, an electron transport layer, and an electron injection layer) in the present invention will be described.

The method for forming the organic layer is not particularly limited, and a conventionally known forming method such as a vacuum vapor deposition method or a wet method (also referred to as a wet process) may be used.

Examples of the wet method include printing methods such as a gravure printing method, a flexographic printing method, and a screen printing method. Further examples of the wet process include: a spin coating method, a cast method, an inkjet printing method, a die coating method, a blade coating method, a bar coating method, a roll coating method, a dip coating method, a spray coating method, a curtain coating method, a doctor coating method, and a LB method (Langmuir Blodgett method). From the viewpoint of easy and accurate coating of the coating liquid with high productivity, it is more preferable to apply by an inkjet printing method using an inkjet head.

A different film forming method may be applied to every organic layer. When a vapor deposition method is adopted for forming each layer, the vapor deposition conditions may be changed depending on the compounds used. Generally, the following ranges are suitably selected for the conditions, heating temperature of boat: 50 to 450° C., level of vacuum: 10⁻⁶ to 10⁻² Pa, vapor deposition rate: 0.01 to 50 nm/sec, temperature of substrate: −50 to 300° C., and layer thickness: 0.1 nm to 5 μm, preferably 5 to 200 nm.

Formation of each organic layer in the present invention is preferably continuously carried out from a hole injection layer to a cathode with one time vacuuming. It may be taken out on the way, and a different layer forming method may be employed. In that case, the operation is preferably done under a dry inert gas atmosphere.

<<Inkjet Printing Method>>

Hereinafter, an example of a forming method of an organic layer by an inkjet printing method will be described with reference to the drawings.

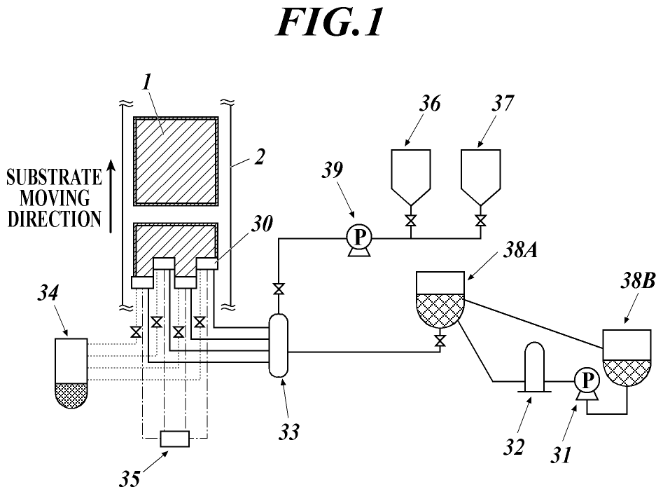

FIG. 1 is a schematic view showing an example of a method for producing an organic EL element using an inkjet printing method.

FIG. 1 shows an example of a method of ejecting an organic functional material (when needed it may contain the π-conjugated compound of the preset invention) that forms an organic layer of an organic EL element onto a base material 2 using an inkjet printing apparatus provided with an inkjet head 30.

As shown in FIG. 1, as an example, while continuously transporting the base material 2, an organic functional material is sequentially ejected onto the base material 2 as ink droplets by the inkjet head 30 to form an organic functional layer of an organic EL element 1.

The inkjet head 30 applicable to the method of producing an organic EL element of the present invention is not particularly limited. For example, it may be a shear mode type (piezo type) head which has a vibration plate having a piezoelectric element in the ink pressure chamber, and an ink liquid is discharged by a pressure change of an ink pressure chamber by the vibration plate, or it may be a thermal type head in which a heating element is provided, and ink liquid is discharged from a nozzle by a rapid volume change due to film boiling of an ink composition due to heat energy from the heating element.

The inkjet head 30 is connected to a supply mechanism of an ink liquid for injection. The ink composition is supplied to the inkjet head 30 by a tank 38A. In this example, the tank composition level is kept constant so that the ink composition pressure in the inkjet head 30 is always kept constant. In this method, the ink liquid is overflowed from the tank 38A and returned to a tank 38B under natural flow. The supply of the ink liquid from the tank 38B to the tank 38A is performed by a pump 31, and is controlled so that the liquid level of the tank 38A is stably constant in accordance with the injection condition.

When returning the ink composition to the tank 38A by the pump 31, it is performed after passing through a filter 32. Thus, the ink composition is preferably passed at least once through a filter medium having an absolute or quasi-absolute filtration accuracy of 0.05 to 50 μm before being supplied to the inkjet head 30.

Further, in order to perform a wash operation or a liquid filling operation of the inkjet head 30, the ink composition may be forcibly supplied from a tank 36 and the cleaning solvent may be forcibly supplied from a tank 37 to the inkjet head 30 by a pump 39. Such tank pumps may be divided into a plurality with respect to the inkjet head 30, a branch of the pipe may be used, or a combination thereof may be used.

In FIG. 1, a piping branch 33 is used. Further, in order to sufficiently remove the air in the inkjet head 30, the ink composition may be extracted from the air extinction pipe described below and sent to a waste liquid tank 34 while forcibly sending the ink liquid from the tank 36 to the inkjet head 30 by the pump 39.

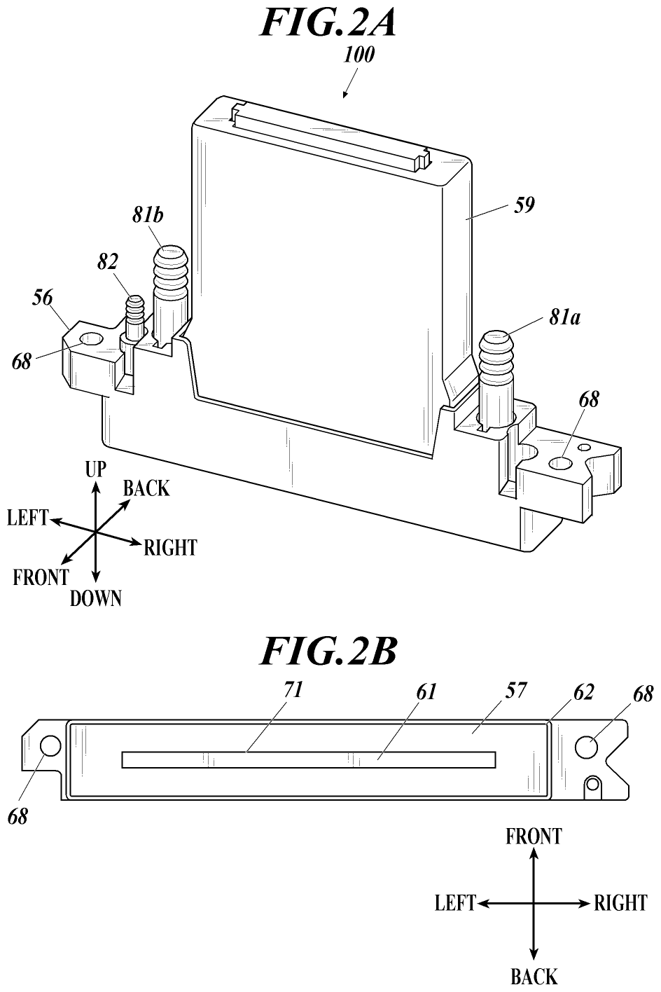

FIG. 2 is a schematic external view showing an example of the structure of an inkjet head applicable to an inkjet printing method.

FIG. 2A is a schematic perspective view showing an inkjet head 100 applicable to the present invention, and FIG. 2B is a bottom view of the inkjet head 100.

The inkjet head 100 applicable to the present invention is mounted on an inkjet printer (not shown). The inkjet head is provided with a head chip for ejecting ink from the nozzle, a wiring board on which the head chip is disposed, a drive circuit board connected to the wiring board through the flexible substrate, a manifold for introducing ink through a filter to the channel of the head chip, a housing 56 in which the manifold is housed inside, a cap receiving plate 57 mounted so as to close the bottom opening of the housing 56, first and second joints 81a, 81b attached to the first ink port and the second ink port of the manifold, a third joint 82 attached to the third ink port of the manifold, and a cover member 59 attached to the housing 56. Further, mounting holes 68 for mounting the housing 56 on the printer main body side are respectively formed.

Further, the cap receiving plate 57 shown in FIG. 2B is formed in a substantially rectangular plate shape having an outer shape elongated in the left-right direction in correspondence with the shape of the cap receiving plate attachment portion 62, and is formed in a substantially central portion thereof in order to expose the nozzle plate 61 on which the plurality of nozzles are arranged, an elongated nozzle opening 71 is provided in the left-right direction. Further, with respect to the specific structure of the inside of the inkjet head shown in FIG. 2A, for example, it is possible to refer to FIG. 2 described in JP-A 2012-140017.

Although a typical example of an inkjet head is shown in FIG. 2, an inkjet head having a configuration described in, for example, JP-A 2012-140017, JP-A 2013-010227, JP-A 2014-058171, JP-A 2014-097644, JP-A 2015-142979, JP-A 2015-142980, JP-A 2016-002675, JP-A 2016-107401, JP-A 2017-109476, and JP-A 2017-177626 may be appropriately selected and applied.

<<Inkjet Head>>

As an ink jet heads that is applicable to the present invention, an ink jet head having a configuration described in the following publications may be appropriately selected and applied. Examples of the publication are JP-A 2012-140017, JP-A 2013-010227, JP-A 2014-058171, JP-A 2014-097644, JP-A 2015-142979, and JP-A 2015-142980, JP-A2016-002675, JP-A 2016-002682, JP-A 2016-107401, JP-A 2017-109476, and JP-A 2017-177626.

The coating liquid used in the wet method may be a solution in which the material forming the organic layer is uniformly dissolved in the liquid medium, or a dispersion liquid in which the material is dispersed in the liquid medium as a solid content. As a dispersion method, dispersion can be performed by a dispersion method such as ultrasonic waves, high shear force dispersion, or media dispersion.

There are no particular restrictions on the liquid medium. Examples of the liquid medium include: halogen solvents such as chloroform, carbon tetrachloride, dichloromethane, 1,2-dichloroethane, dichlorobenzene and dichlorohexanone, ketone solvents such as acetone, methyl ethyl ketone, diethyl ketone, methyl isobutyl ketone, n-propyl methyl ketone, and cyclohexanone; aromatic solvents such as benzene, toluene, xylene, mesitylene, cyclohexylbenzene; aliphatic solvents such as cyclohexane, decalin, and dodecane; ester solvents such as ethyl acetate, n-propyl acetate, n-butyl acetate, methyl propionate, ethyl propionate, γ-butyrolactone, and diethyl carbonate; ether-based solvents such as tetrahydrofuran and dioxane; amide-based solvents such as dimethylformamide and dimethylacetamide; alcohol-based solvents such as methanol, ethanol, 1-butanol and ethylene glycol; nitrile-based solvents such as acetonitrile and propionitrile; dimethyl sulfoxide, water and a mixed solution medium thereof.

The boiling point of these liquid media is preferably a boiling point lower than the temperature of the drying treatment from the viewpoint of quickly drying the liquid medium, specifically in the range of 60 to 200° C., and more preferably in the range of 80 to 180° C.

The coating liquid may contains a surfactant depending on the purpose of controlling the coating range and suppressing the liquid flow (for example, the liquid flow that causes a phenomenon called a coffee ring) associated with the surface tension gradient after coating.

Examples of the surfactant include anionic or nonionic surfactants from the viewpoints of the influence of water contained in the solvent, leveling property, wettability to the substrate f1. Specifically, fluorine-containing surfactants and the surfactants listed in WO 08/146681, JP-A2-41308 may be used.

As with the film thickness, the viscosity of the coating film may be appropriately selected depending on the function required as the organic layer and the solubility or dispersibility of the organic material. Specifically, for example, it may be selected within the range of 0.3 to 100 mPa·s.

The film thickness of the coating film may be appropriately selected depending on the function required as the organic layer and the solubility or dispersibility of the organic material. Specifically, it may be selected in the range of, for example, 1 to 90 μm.

After forming the coating film by the wet method, it is possible to have a coating step of removing the above-mentioned liquid medium. The temperature of the drying step is not particularly limited, but it is preferable to perform the drying treatment at a temperature that does not damage the organic layer, the transparent electrode, or the base material. Specifically, it may not be said unconditionally because it differs depending on the composition of the coating liquid, but for example, the temperature may be set to 80° C. or higher, and the upper limit is considered to be a possible range up to about 300° C. The drying tune is preferably about 10 seconds or more and 10 minutes or less. Under such conditions, drying may be performed quickly.

<<Sealing>>

As sealing means employed for sealing an organic EL element, listed may be, for example, a method in which sealing members, electrodes, and a support substrate are subjected to adhesion via adhesives. The sealing members may be arranged to cover the display region of an organic EL element, and may be a concave plate or a flat plate. Neither transparency nor electrical insulation is limited.

Specifically listed are glass plates, polymer plate-films, metal plate-films. Specifically, it is possible to list, as glass plates, soda-lime glass, barium-strontium containing glass, lead glass, aluminosilicate glass, borosilicate glass, barium borosilicate glass, and quartz. Further, listed as polymer plates may be polycarbonate, actyl, polyethylene terephthalate, polyether sulfide, and polysulfone. As a metal plate, listed are those composed of at least one metal selected from the group consisting of stainless steel, iron, copper, aluminum magnesium, nickel, zinc, chromium, titanium, molybdenum, silicon, germanium, and tantalum, or alloys thereof.

In the present invention, since it is possible to achieve a thin organic EL element, it is preferable to employ a polymer film or a metal film. Further, it is preferable that the polymer film has an oxygen permeability of 1×10⁻³ mL/m²/24 h or less determined by the method based on JIS K 7126-1987, and a water vapor permeability (at 25±0.5° C. relative humidity of (90±2)%) of 1×10⁻³ g/(m²·24 h) or less determined by the method based on JIS K 7129-1992.

Conversion of the sealing member into concave is carried out by employing a sand blast process or a chemical etching process.

In practice, as adhesives, listed may be photo-curing and heat-curing types having a reactive vinyl group of acrylic acid based oligomers and methacrylic acid, as well as moisture curing types such as 2-cyanoacrylates. Further listed may be thermal and chemical curing types (mixtures of two liquids) such as epoxy based ones. Still further listed may be hot-melt type polyamides, polyesters, and polyolefins. Yet further listed may be cationically curable type UV curable epoxy resin adhesives.

In addition, since an organic EL element is occasionally deteriorated via a thermal process, preferred are those which enable adhesion and curing between room temperature and 80° C. Further, desiccating agents may be dispersed into the aforesaid adhesives. Adhesives may be applied onto sealing portions via a commercial dispenser or printed on the same in the same manner as screen printing.

Further, it is appropriate that on the outside of the aforesaid electrode which interposes the organic layer and faces the support substrate, the aforesaid electrode and organic layer are covered, and in the form of contact with the support substrate, inorganic and organic material layers are formed as a sealing firm in this case, as materials that form the aforesaid film may be those which exhibit functions to retard penetration of moisture or oxygen which results in deterioration. For example, it is possible to employ silicon oxide, silicon dioxide, and silicon nitride.

Still further, in order to improve brittleness of the aforesaid film, it is preferable that a laminated layer structure is formed, which is composed of these inorganic layers and layers composed of organic materials. Methods to form these films are not particularly limited. It is possible to employ, for example, a vacuum deposition method, a sputtering method, a reactive sputtering method, a molecular beam epitaxy method, a cluster ion beam method, an ion plating method, a plasma polymerization method, an atmospheric pressure plasma polymerization method, a plasma CVD method, a thermal CVD method, and a coating method.

It is preferable to inject a gas phase material or a liquid phase material, for example, an inert gas such as nitrogen or argon, or an inactive liquid such as fluorinated hydrocarbon or silicone oil into the space formed between the sealing member and the display region of the organic EL element. Further, it is possible to form vacuum in the space. Still further, it is possible to enclose hygroscopic compounds in the interior of the space.

Examples of a hygroscopic compound include: metal oxides (for example, sodium oxide, potassium oxide, calcium oxide, barium oxide, magnesium oxide, and aluminum oxide); sulfates (for example, sodium sulfate, calcium sulfate, magnesium sulfate, and cobalt sulfate); metal halides (for example, calcium chloride, magnesium chloride, cesium fluoride, tantalum fluoride, cerium bromide, magnesium bromide, barium iodide, and magnesium iodide); perchlorates (for example, barium perchlorate and magnesium perchlorate). For sulfate salts, metal halides and perchlorates, suitably used are anhydrous salts.

<<Protective Film and Protective Plate>>

On the aforesaid sealing film which interposes the organic layer and faces the support substrate or on the outside of the aforesaid sealing film, a protective or a protective plate may be arranged to enhance the mechanical strength of the element. Specifically, when scaling is achieved via the aforesaid sealing film, the resulting mechanical strength is not always high enough, therefore it is preferable to arrange the protective film or the protective plate described above. Usable materials for these include glass plates, polymer plate-films, and metal plate-films which are similar to those employed for the aforesaid sealing. However, from the viewpoint of reducing weight and thickness, it is preferable to employ a polymer film.

<<Improving Method of Light Extraction>>

It is generally known that an organic EL element emits light in the interior of the layer exhibiting the refractive index (being about 1.6 to 2.1) which is greater than that of air, whereby only about 15% to 20% of light generated in the light-emitting layer is extracted. This is due to the fact that light incident to an interface (being an interlace of a transparent substrate to air) at an angle of θ which is at least critical angle is not extracted to the exterior of the element due to the resulting total reflection, or light is totally reflected between the transparent electrode or the light-emitting layer and the transparent substrate, and light is guided via the transparent electrode or the light-emitting layer, whereby light escapes in the direction of the element side surface.

Means to enhance the efficiency of the aforesaid light extraction include, for example: a method in which roughness is formed on the surface of a transparent substrate, whereby total reflection is minimized at the interface of the transparent substrate to air (U.S. Pat. No. 4,774,435), a method in which efficiency is enhanced in such a manner that a substrate results in light collection (JP-A 63-314795), a method in which a reflection surface is formed on the side of the element (JP-A 1-220394), a method in which a flat layer of a middle refractive index is introduced between the substrate and the light-emitting body and an antireflection film is formed (JP-A 62-172691), a method in which a flat layer of a refractive index which is equal to or less than the substrate is introduced between the substrate and the light-emitting body (JP-A 2001-202827), and a method in which a diffraction grating is formed between the substrate and any of the layers such as the transparent electrode layer or the light-emitting layer (including between the substrate and the outside) (JP-A 11-283751).

In the present invention, it is possible to employ these methods while combined with the organic EL element of the present invention. Of these, it is possible to appropriately employ the method in which a flat layer of a refractive index which is equal to or less than the substrate is introduced between the substrate and the light-emitting body and the method in which a diffraction grating is formed between any layers of a substrate, and a transparent electrode layer and a light-emitting layer (including between the substrate and the outside space).

When a low refractive index medium having a thickness, greater than the wavelength of light is formed between the transparent electrode and the transparent substrate, the extraction efficiency of light emitted from the transparent electrode to the exterior increases as the refractive index of the medium decreases.

By combining these means, the present, invention enables the production of elements which exhibit higher luminance or excel in durability.

As materials of the low refractive index layer, listed are, for example, aerogel, porous silica, magnesium fluoride, and fluorine based polymers. Since the refractive index of the transparent substrate is commonly about 1.5 to 1.7, the refractive index of the low refractive index layer is preferably approximately 1.5 or less. More preferably, it is 1.35 or less.

Further, thickness of the low refractive index medium is preferably at least two times of the wavelength in the medium. The reason is that, when the thickness of the low refractive index medium reaches nearly the wavelength of light so that electromagnetic waves escaped via evanescent enter into the substrate, effects of the low refractive index layer are lowered.

The method in which the interface which results in total reflection or a diffraction grating is introduced in any of the media is characterized in that light extraction efficiency is significantly enhanced. The above method works as follows. By utilizing properties of the diffraction grating capable of changing the light direction to the specific direction different from diffraction via so-called Bragg diffraction such as primary diffraction or secondary diffraction of the diffraction grating, of light emitted from the light entitling layer, light, which is not emitted to the exterior due to total reflection between layers, is diffracted via introduction of a diffraction grating between any layers or in a medium (in the transparent substrate and the transparent electrode) so that light is extracted to the exterior.

It is preferable that the introduced diffraction grating exhibits a two-dimensional periodic refractive index. The reason is as follows. Since light emitted in the light-emitting layer is randomly generated to all directions, in a common one-dimensional diffraction grating exhibiting a periodic refractive index distribution only in a certain direction, light which travels to the specific direction is only diffracted, whereby light extraction efficiency is not sufficiently enhanced.

However, by changing the refractive index distribution to a two-dimensional one, light, which travels to all directions, is diffracted, whereby the light extraction efficiency is enhanced.

A position to introduce a diffraction grating may be between any layers or in a medium (in a transparent substrate or a transparent electrode). However, a position near the organic light-emitting layer, where light is generated, is preferable. In this case, the cycle of the diffraction grating is preferably from about ½ to 3 times of the wavelength of light in the medium. The preferable arrangement of the diffraction grating is such that the arrangement is two-dimensionally repeated in the form of a square lattice, a triangular lattice, or a honeycomb lattice.

<<Light Collection Sheet>>

Via a process to arrange a structure such as a micro-lens array shape on the light extraction side of the support substrate (substrate) of the organic EL element of the present invention or via combination with a so-called light collection sheet, light is collected in the specific direction such as the front direction with respect to the light-emitting element surface, whereby it is possible to enhance luminance in the specific direction.

In an example of the micro-lens array, square pyramids to realize a side length of 30 μm and an apex angle of 90 degrees are two-dimensionally arranged on the light extraction side of the substrate. The side length is preferably 10 to 100 μm. When it is less than the lower limit, coloration occurs due to generation of diffraction effects, while when it exceeds the upper limit, the thickness increases undesirably.

It is possible to employ, as a light collection sheet, for example, one which is put into practical use in the LED backlight of liquid crystal display devices. It is possible to employ, as such a sheet, for example, the luminance enhancing film (BEF), produced by Sumitomo 3M Limited. As shapes of a prism sheet employed may be, for example, Δ-shaped stripes of an apex angle of 90 degrees and a pitch of 50 μm formed on a substrate, a shape in which the apex angle is rounded, a shape in which the pitch is randomly changed, and other shapes.

Further, in order to control the light radiation angle from the light-emitting element, simultaneously employed may be a light diffusion plate-film. For example, it is possible to employ the diffusion film (LIGHT-UP), produced by Kimoto Co., Ltd.

<<Applications>>

It is possible to employ the organic EL element of the present invention as display devices, displays, and various types of light-emitting sources.

Examples of light-emitting sources include: lighting devices (home lighting and car lighting), clocks, backlights for liquid crystals, sign advertisements, signals, light sources of light memory media, light sources of electrophotographic copiers, light sources of light communication processors, and light sources of light sensors. The present invention is not limited to them. It is especially effectively employed as a backlight of a liquid crystal display device and a lighting source.

If needed, the organic EL element of the present invention may undergo patterning via a metal mask or an ink-jet printing method during film formation. When the patterning is carried out, only an electrode may undergo patterning, an electrode and a light-emitting layer may undergo patterning, or all element layers may undergo patterning. During preparation of the element, it is possible to employ conventional methods.

<<Embodiment of Lighting Device>>

An aspect of the lighting device provided with the organic EL element of the present invention will be described.

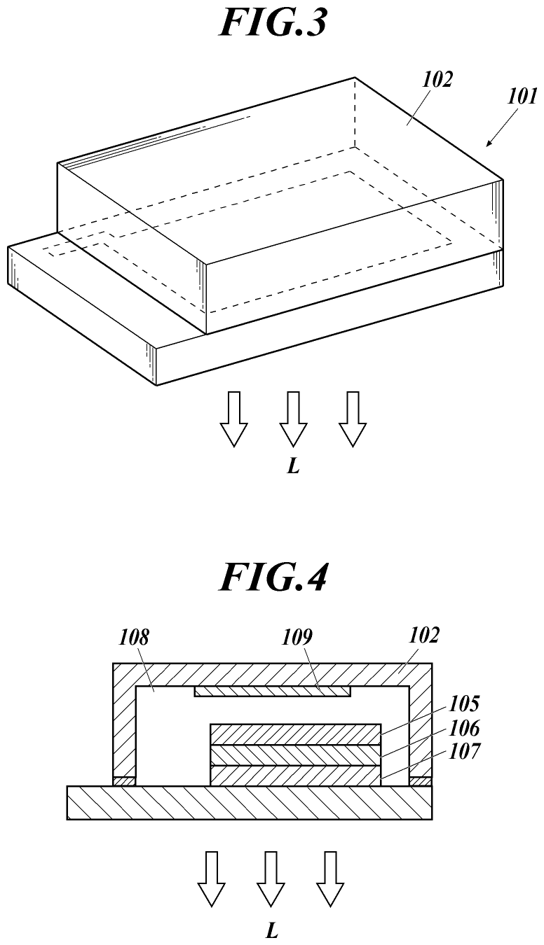

The non-light-emitting surface of the organic EL element of the present invention is covered with a glass case, and a 300 μm thick glass substrate is employed as a sealing substrate. An epoxy based light curable type adhesive (LUXTRACK LC0629B produced by Toagosei Co., Ltd.) is employed in the periphery as a sealing material. The resulting one is superimposed on the aforesaid cathode to be brought into close contact with the aforesaid transparent support substrate, and curing and sealing are carried out via exposure of UV radiation onto the glass substrate side, whereby the lighting device shown in FIG. 3 and FIG. 4 is formed.

FIG. 3 is a schematic view of a lighting device. An organic EL element 101 of the present invention is covered with a glass cover 102 (incidentally, sealing by the glass cover was carried out in a globe box under nitrogen ambience (under air ambience of high purity nitrogen gas at a purity of at least 99.999%) so that the organic EL Element 101 was not brought into contact with atmosphere.

FIG. 4 is a cross-sectional view of a lighting device. In FIG. 4, the reference sign 105 represents a cathode, the reference sign 106 represents an organic EL layer, and the reference sign 107 represents a glass substrate having a transparent electrode. Further, the interior of glass cover 102 is filled with nitrogen gas 108 and water catching agent 109 is provided.

[Light-Emitting Thin Film]

The light-emitting film of the present invention contains the above-described π-conjugated compound.

The light-emitting thin film of the present invention may be produced in the same manner as the method for forming the organic layer (light-emitting layer).

The method for forming the light-emitting thin film of the present invention is not particularly limited, and a conventionally known forming method such as a vacuum vapor deposition method or a wet method (also referred to as a wet process) may be used.

Examples of the wet method include a spin coating method, a casting method, an inkjet method, a printing method, a die coating method, a blade coating method, a roll coating method, a spray coating method, a curtain coating method, and an LB (Langmuir-Blodgett) method. From the viewpoint of easily obtaining a uniform thin film and high productivity, a method having high suitability for a roll-to-roll method such as a die coating method, a roll coating method, an inkjet method and a spray coating method is preferable.

Examples of a liquid medium used for forming a light-emitting thin film of the present invention include: ketones such as methyl ethyl ketone and cyclohexanone; aliphatic esters such as ethyl acetate; halogenated hydrocarbons such as dichlorobenzene; aromatic hydrocarbons such as toluene, xylene, mesitylene, and cyclohexylbenzene; aliphatic hydrocarbons such as cyclohexane, decalin, and dodecane; organic solvents such as DMF and DMSO.

These will be dispersed with a dispersion method such as an ultrasonic dispersion method, a high shearing dispersion method and a media dispersion method.

When a vapor deposition method is adopted for forming each layer, the vapor deposition conditions may be changed depending on the compounds used. Generally, the following ranges are suitably selected for the conditions, heating temperature of boat: 50 to 450° C., level of vacuum: 10⁻⁶ to 10⁻² Pa, vapor deposition rate: 0.01 to 50 nm/sec, temperature of substrate: −50 to 300° C., and layer thickness: 0.1 nm to 5 μm, preferably 5 to 200 nm.

When a spin coating method is adopted for filth formation, it is preferable to operate the spin coater in the range of 100 to 1000 rpm and in the range of 10 to 120 seconds in a dry inert gas atmosphere.

[Ink Composition]

The ink composition of the present invention contains the above-described π-conjugated compound.

By containing the π-conjugated compound, it is possible to prepare a composition capable of suppressing fluctuations in physical properties of the charge transfer/light-emitting thin film using the composition over time of energization, improving luminous efficiency and improve the life of the luminescent element, and having a deep blue color.

Examples of the coating method of the ink composition of the present invention include printing methods such as a gravure printing method, a flexographic printing method, and a screen printing method. Further examples of the coating method include: a spin coating method, a cast method, an inkjet printing method, a die coating method, a blade coating method, a bar coating method, a roll coating method, a dip coating method, a spray coating method, a curtain coating method, a doctor coating method, and a LB method (Langmuir Blodgett method). From the viewpoint of easy and accurate coating of the ink composition with high productivity, it is more preferable to apply by an inkjet printing method using an inkjet head.

Regarding the method of dispersing the ink composition in the liquid medium, the type of the liquid medium, the surfactant contained in the ink composition, and the viscosity and film thickness of the coating film to which the ink composition is applied, they are explained in the item of “Production method of organic EL element”.

Further, the ink composition of the present invention is used as an organic EL element material.

[Organic EL Element Material]

The organic EL element material of the present invention contains the π-conjugated compound.

By containing the π-conjugated compound, it is possible to produce an organic EL element capable of suppressing fluctuations in physical properties of the charge transfer/light-emitting thin film using the organic EL element material over time of energization, improving luminous efficiency and improve the life of the light-emitting element, and emitting deep blue color.

The organic EL element material of the present invention may be used as a material for the organic layer of the organic EL element described above. It may be used as the material for a light-emitting layer, an electron transport layer, a hole blocking layer, an electron injection layer, a hole transport layer, an electron blocking layer, and a hole injection layer.

[Light-Emitting Material]

The light emitting material of the present invention contains the above-described π-conjugated compound, and the π-conjugated compound emits fluorescence. That is, the π-conjugated compound is contained as a light-emitting material used for the light-emitting layer.

Further, in the light-emitting material of the present invention, it is preferable that the π-conjugated compound emits delayed fluorescence.

[Charge Transport Material]

The charge transport material of the present invention contains the above-described π-conjugated compound, and the π-conjugated compound emits fluorescence. That is, the π-conjugated compound is contained as a light-emitting material used for the charge transport layer.

Further, in the charge transport material of the present invention, it is preferable that the π-conjugated compound emits delayed fluorescence.

### EXAMPLES

Hereinafter, the present invention will be specifically described with reference to examples, but the present invention is not limited thereto.

The compounds used in Examples and Comparative Examples are shown below.

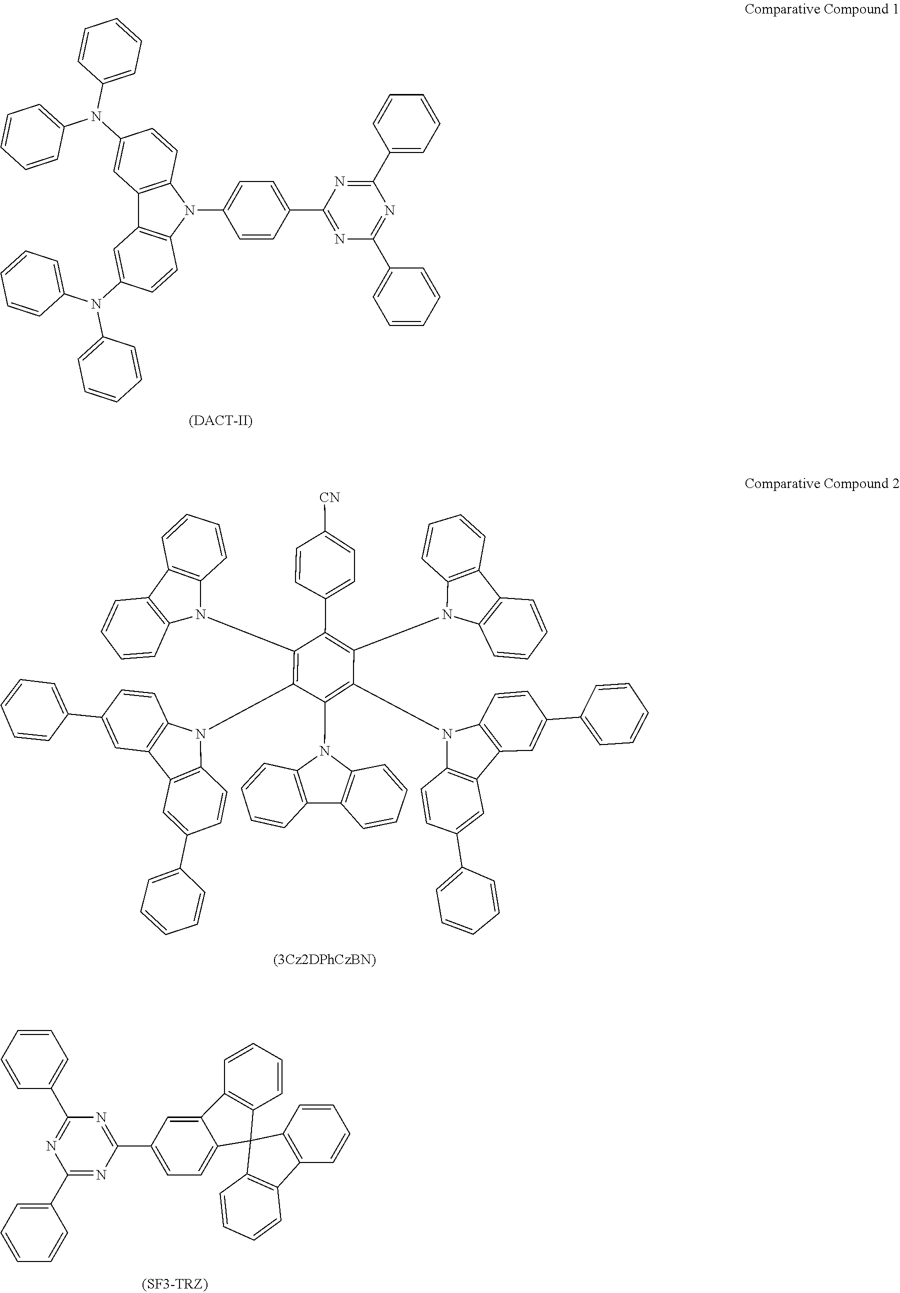

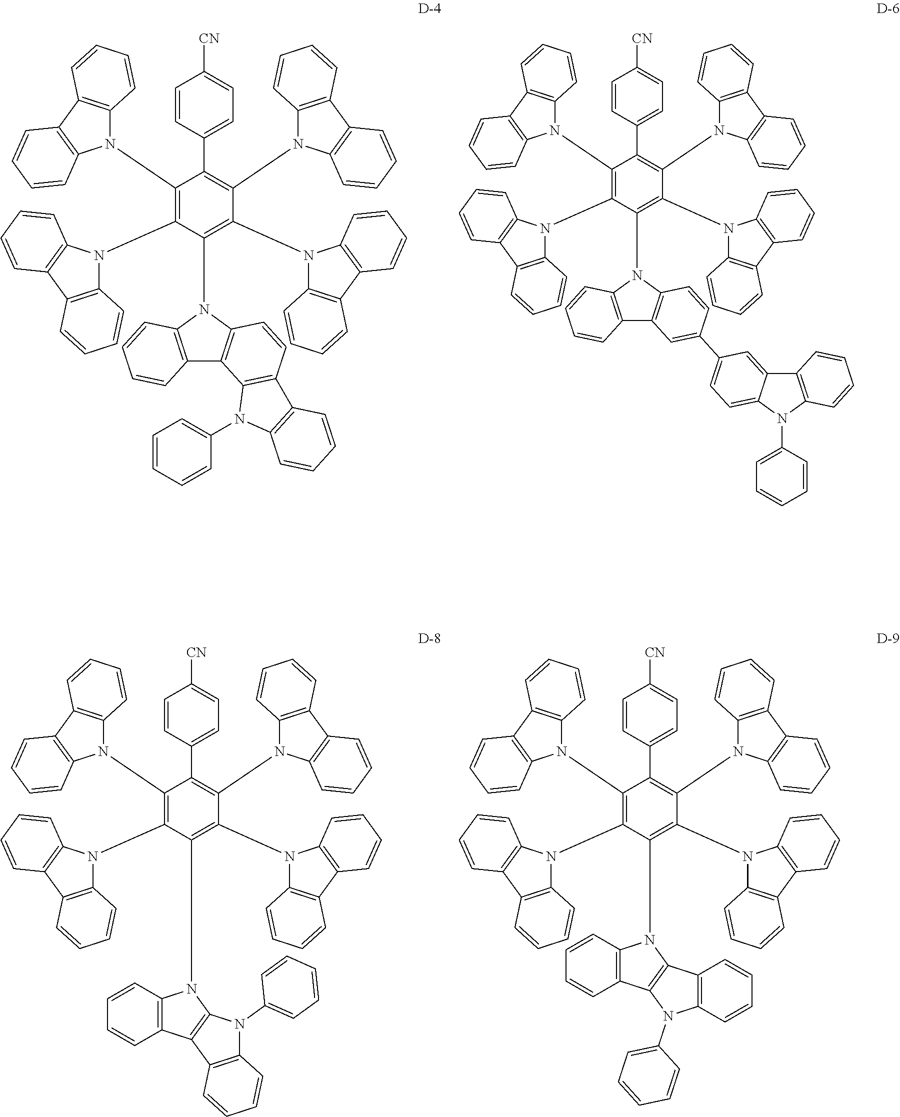

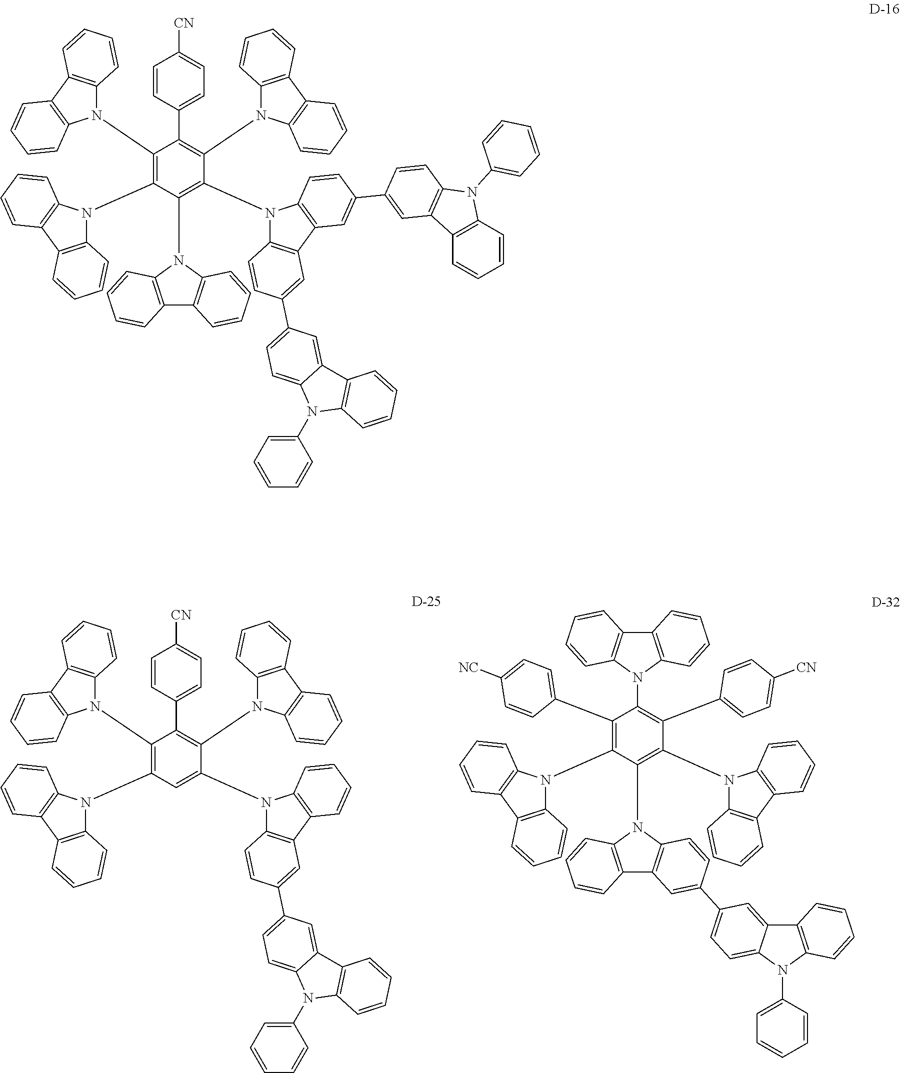 [SMILES: N#Cc1ccc(-c2c(-n3c4ccccc4c4ccccc43)c(-n3c4ccccc4c4ccccc43)c(-n3c4ccccc4c4ccccc43)c(-n3c4ccc(-c5ccc6c(c5)c5ccccc5n6-c5ccccc5)cc4c4cc(-c5ccc6c(c5)c5ccccc5n6-c5ccccc5)ccc43)c2-n2c3ccccc3c3ccccc32)cc1 ; N#Cc1ccc(-c2c(-n3c4ccccc4c4ccccc43)c(-n3c4ccccc4c4ccccc43)cc(-n3c4ccccc4c4cc(-c5ccc6c(c5)c5ccccc5n6-c5ccccc5)ccc43)c2-n2c3ccccc3c3ccccc32)cc1 ; N#Cc1ccc(-c2c(-n3c4ccccc4c4ccccc43)c(-c3ccc(C#N)cc3)c(-n3c4ccccc4c4ccccc43)c(-n3c4ccccc4c4cc(-c5ccc6c(c5)c5ccccc5n6-c5ccccc5)ccc43)c2-n2c3ccccc3c3ccccc32)cc1]

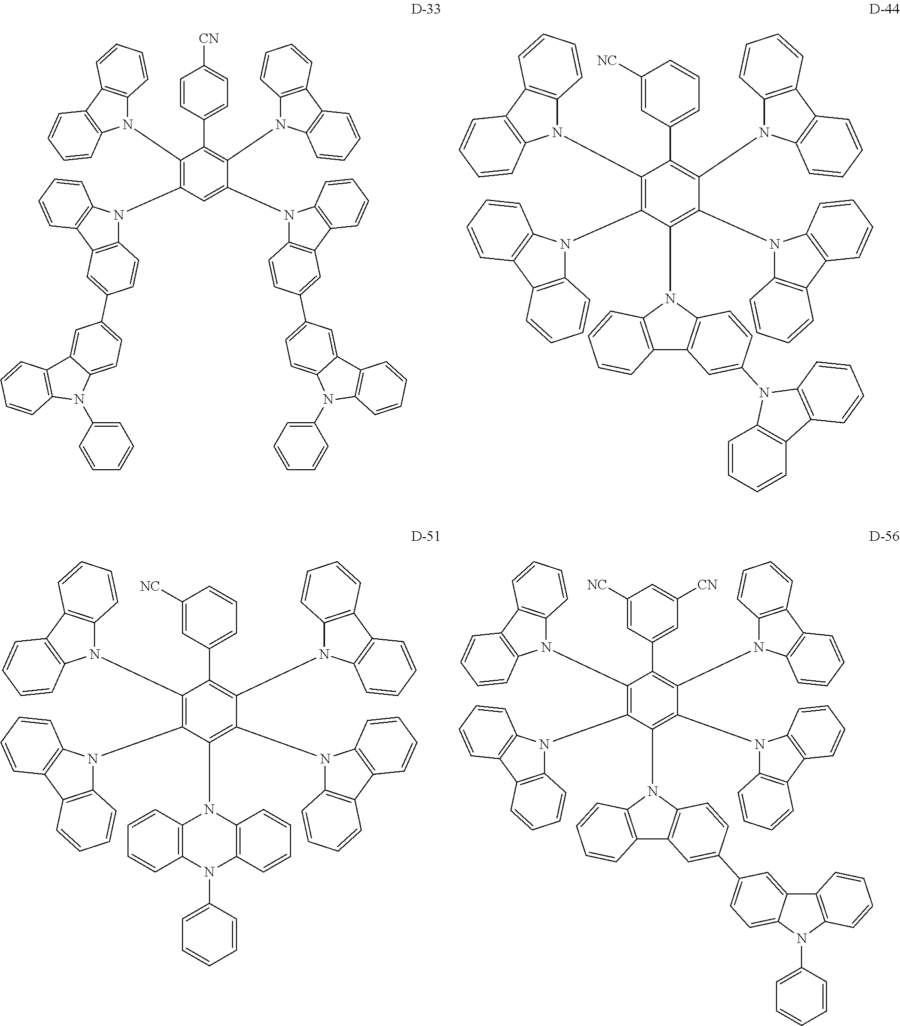

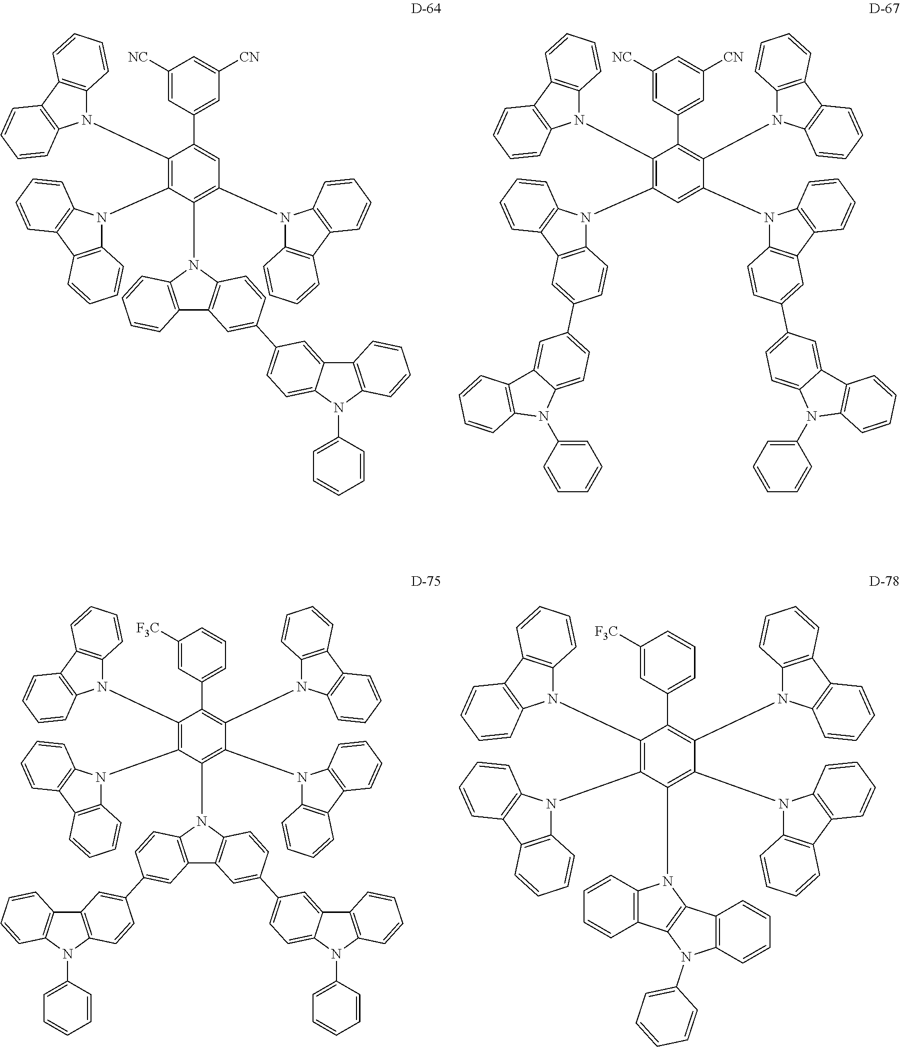

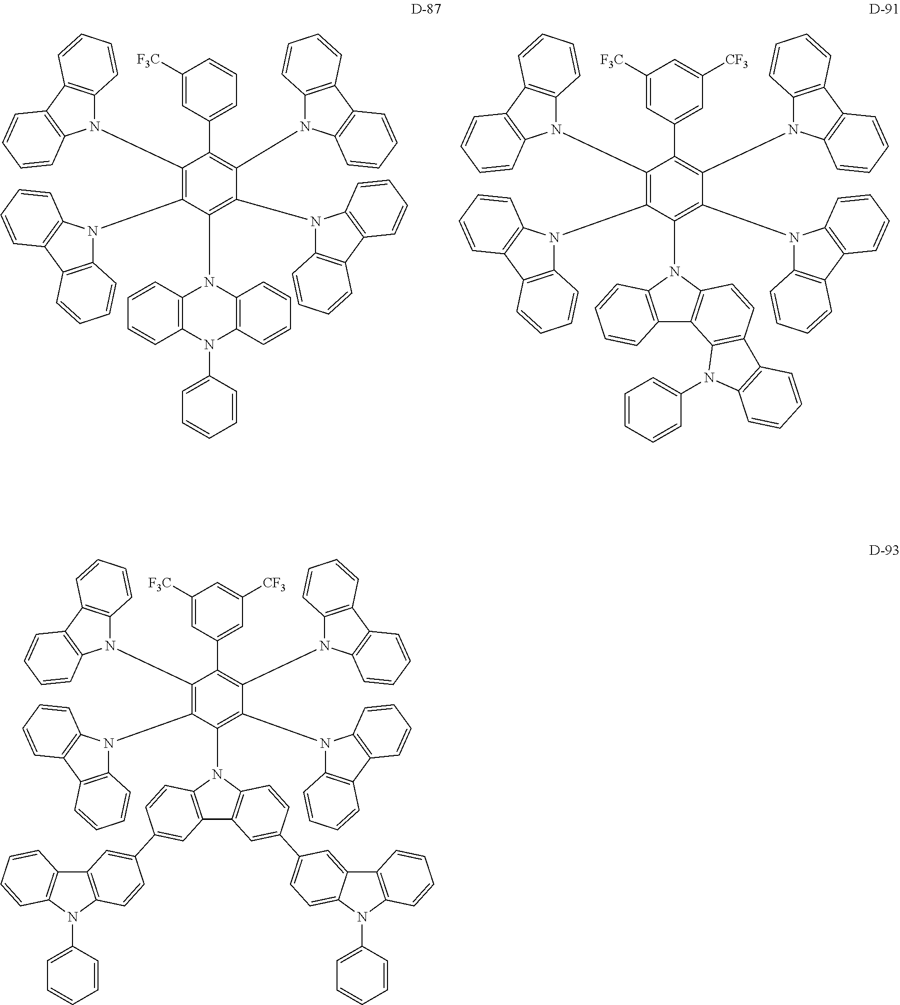

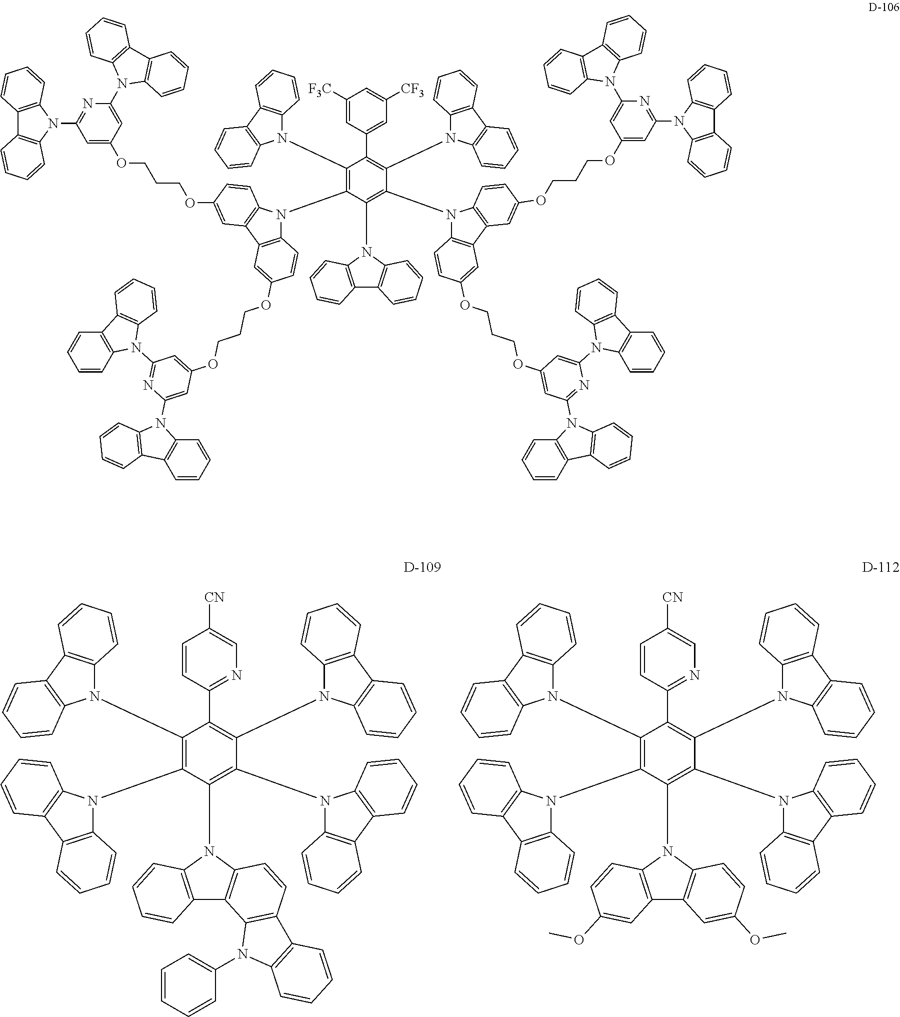

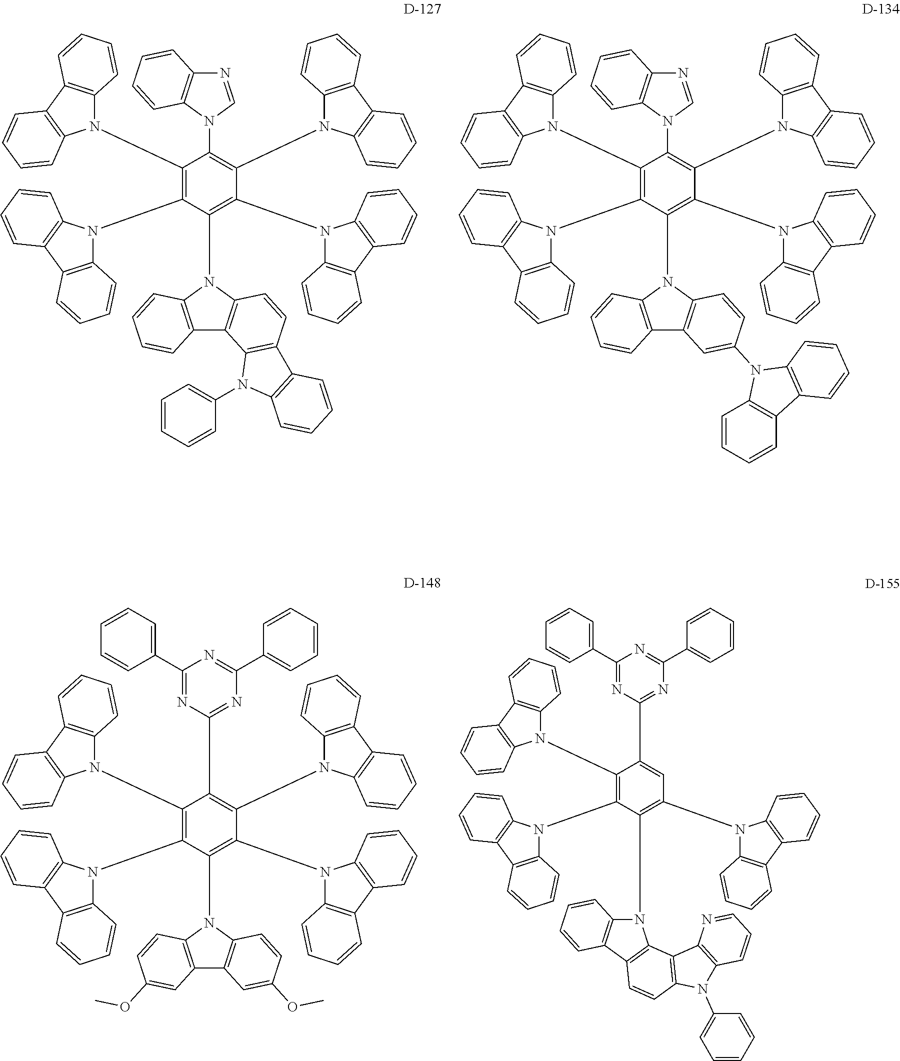

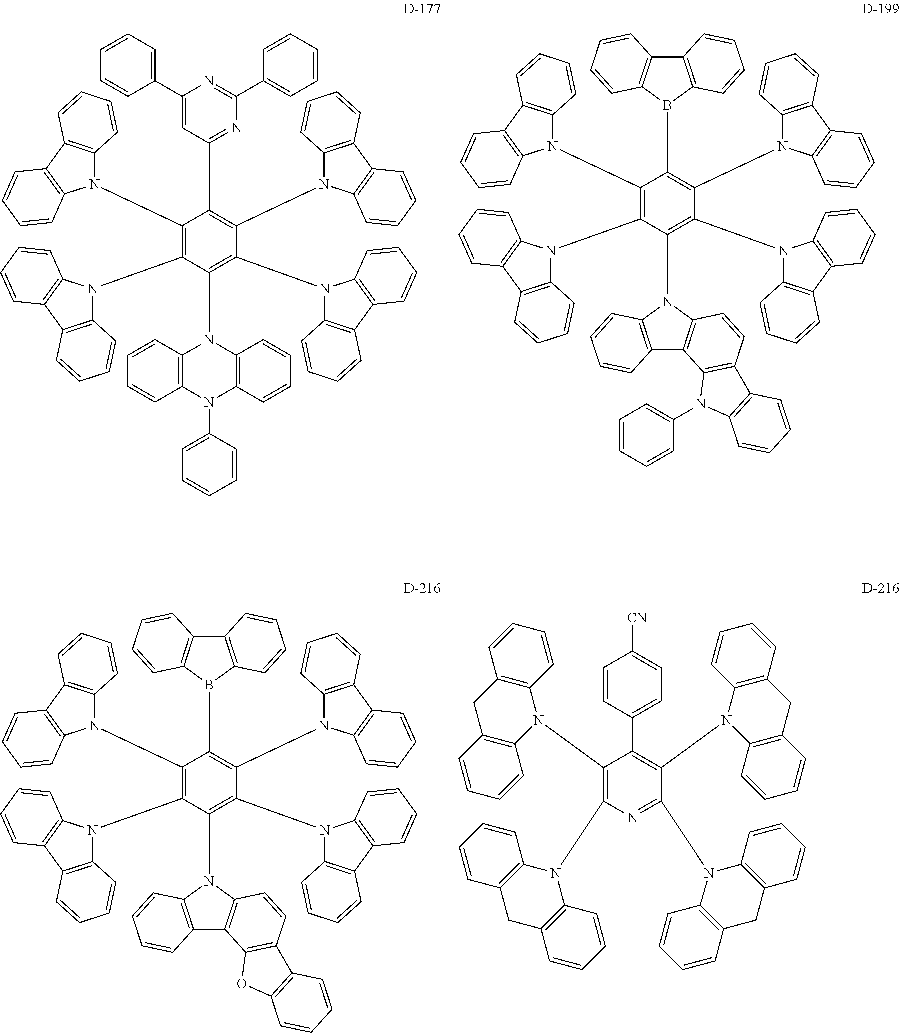

### Example 1

### <Synthesis of Exemplary Compound D-6>

It was synthesized according to the following scheme.

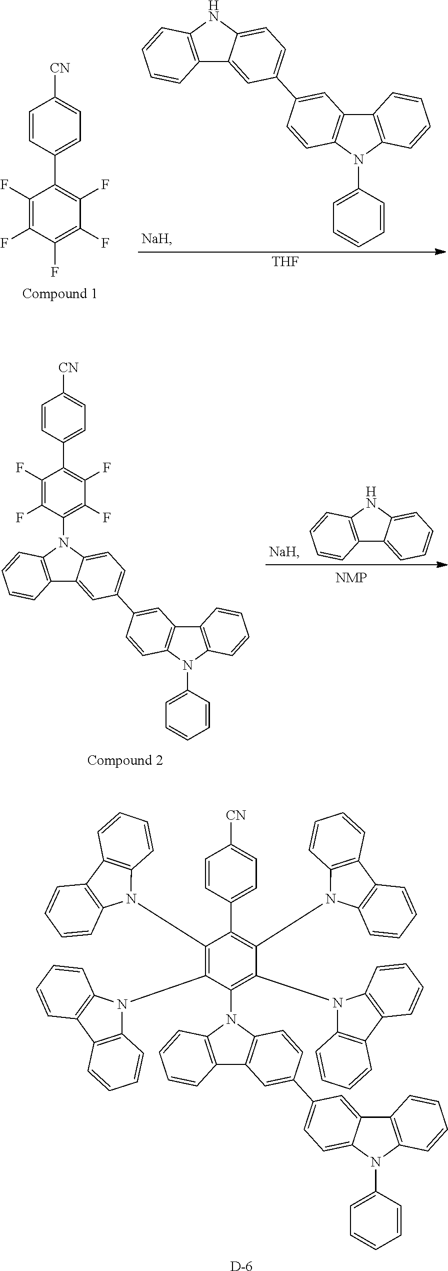

9-Phenyl-9H,9′H-3,3′-bicarbazole (4.41 g, 10.8 mol) was dissolved THF (tetrahydrofuran) (42 ml). NaH (0.48 g, 11.9 mol) was added thereto, and the mixture was stirred for 30 minutes. Thereafter, Compound 1 (2.91 g, 10.8 mol) was added to the solution, and the mixture was stirred for 5 hours with heating under reflux. Water was added to the reaction solution, and the precipitate was collected by filtration. The precipitate was recrystallized to obtain 6.39 g of Compound 2.

Then, carbazole (8.13 g, 48.6 mol) was dissolved in NMP (42 ml). NaH (2.13 g, 53.5 mol) was added thereto, and the mixture was stirred for 30 minutes. Thereafter, Compound 2 (6.39 g, 9.71 mol) was added to the solution, and the mixture was heated and stirred at 120° C., for 5 hours. Water was added to the reaction solution, and the precipitate was collected by filtration. The precipitate was recrystallized to obtain 10.3 g of the target exemplary compound (D-6).

<Synthesis of Exemplary Compound D-33>

It was synthesized according to the following scheme.

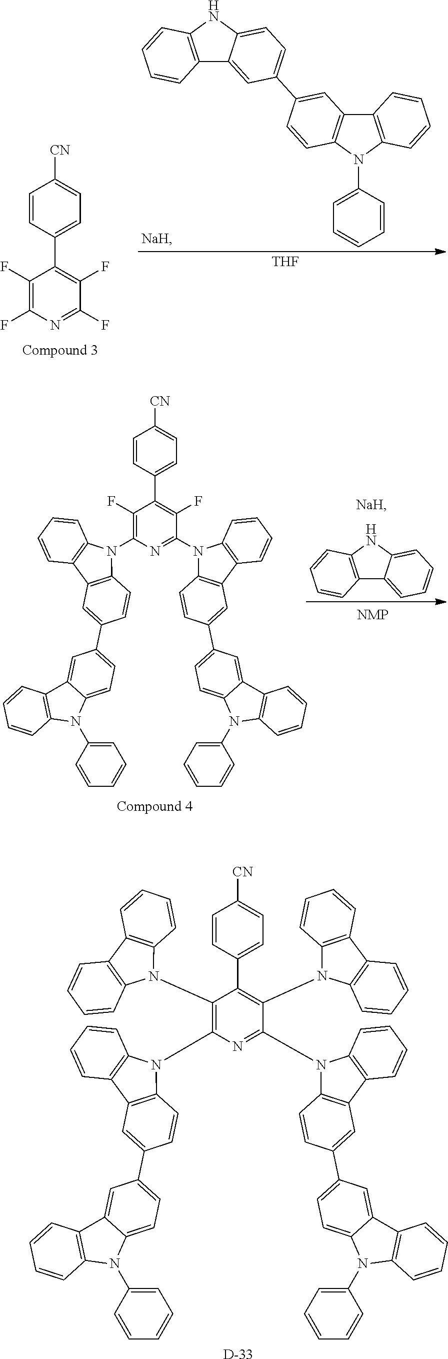

9-Phenyl-9H,9′H-3,3′-bicarbazole (8.82 g, 21.6 mol) was dissolved in THF (tetrahydrofuran) (42 ml). NaH (0.96 g, 23.8 mol) was added thereto, and the mixture was stirred for 30 minutes. Thereafter, Compound 3 (2.72 g, 10.8 mol) was added to the solution, and the mixture was stirred for 5 hours with heating under reflux. Water was added to the reaction solution, and the precipitate was collected by filtration. The precipitate was recrystallized to obtain 9.44 g of Compound 4.

Then, carbazole (3.40 g, 20.3 mol) was dissolved in NMP (42 ml). NaH (0.97 g, 24.4 mol) was added thereto, and the mixture was stirred for 30 minutes. Thereafter, Compound 4 (9.44 g, 9.24 mol) was added to the solution, and the mixture was heated and stirred at 120° C. for 5 hours. Water was added to the reaction solution, and the precipitate was collected by filtration. The precipitate was recrystallized to obtain 11.0 g of the target exemplary compound (D-33).

<Synthesis of Other Exemplary Compounds>

Compounds D-4, D-6, D-8, D-9, D-16, D-25, D-32, D-33, D-44, D-51, D-56, D-64, D-67, D-75, D-78, D-87, D-91, D-93, D-106, D-109, D -112, D-127, D-134, D-148, D-155, D-177, D-199, D-207 and D-216 were synthesized in the same manner as described above except that the starting substrate and the reaction substrate were mainly changed.

The ΔEst and the HOMO level of the obtained exemplary compounds and the comparative compound were calculated by the following method.

<Calculation of ΔEst and HOMO Level>

The structural optimization and the calculation of the electron density distribution by the molecular orbital calculation of the compound were calculated using the molecular orbital calculation software using B3LYP as a functional and 6-31G (d) as a basis function as the calculation method. As a molecular orbital calculation software, it was used Gaussian 09 made by the US Gaussian Inc. (Revision C.01, by M. J. Frisch et al., Gaussian Inc., 2010).

From the structure optimization calculation using B3LYP as this functional and 6-31G (d) as the basis function, the excitation state calculation by the time-dependent density functional theory (Time-Dependent DFT) was further performed to calculate the HOMO level. ΔEst was calculated from ΔEst=|E(S₁)−E(T₁)| by obtaining the energy levels of S₁ and T₁ (E(S₁) and E(T₁), respectively.

### Example 2

<Preparation of Organic EL Element 1-1>

An anode was prepared by making patterning to a glass substrate of 100 mm×100 mm×1.1 mm (NA45, produced by AvanStrate Inc.) on which ITO (indium tin oxide) was formed with a thickness of 100 nm. Thereafter, the above transparent support substrate provided with the ITO transparent electrode was subjected to ultrasonic washing with isopropyl alcohol, followed by drying with desiccated nitrogen gas, and it was subjected to UV ozone washing for 5 minutes.

On the transparent support substrate thus prepared was applied a 70% solution of poly (3,4-ethylenedioxythiphene)-polystyrene sulfonate (PEDOT/PSS, Baytron P AI4083, made by Bayer AG.) diluted with water by using a spin coating method at 3000 rpm for 30 seconds to form a film, and then it was dried at 200° C. for one hour. Thus, a hole injection layer having a thickness of 20 nm was prepared.

Then, a thin film was formed by a spin coating method under the conditions of 2000 rpm and 30 seconds using a solution of polyvinylcarbazole (Mw: about 1100000) in 1,2 dichlorobenzene, and then dried at 120° C. for 10 minutes to form a layer. A hole transport layer having a thickness of 15 nm was provided.

Further, a thin film was prepared by a spin coating method under the conditions of 2000 rpm for 30 seconds using a solution prepared by dissolving comparative compound 1 as a light-emitting compound and mCBP (3,3-di(9H-carbazole-9-yl)biphenyl) as a host compound in toluene so as to be 10% and 90% by mass, respectively. After forming the thin film, it was dried at 100° C. for 10 minutes to provide a light-emitting layer having a layer thickness of 35 nm.

Then, the resulting transparent support substrate was fixed to a substrate holder of a commercial vacuum deposition apparatus.

The constituting materials for each layer were loaded in each crucible for vapor deposition in the vacuum deposition apparatus with an optimum amount. As the crucible for vapor deposition, a crucible made of molybdenum or tungsten made of a resistance heating material was used.

After reducing the pressure to a vacuum degree of 1×10⁻⁴ Pa, SF3-TRZ was vapor-deposited at a vapor deposition rate of 1.0 nm/sec to form a hole blocking layer having a layer thickness of 5 nm.

Then, SF3-TRZ and LiQ (8-hydroxyquinolinolato-lithium) were co-deposited at a vapor deposition rate of 1.0 nm/sec so as to be 50 mol % and 50 mol %, respectively, and an electron transport layer with a layer thickness of 30 min was formed.

Further, alter forming lithium fluoride with a film thickness of 0.5 nm, aluminum was vapor-deposited with a layer thickness of 100 nm to form a cathode.

The non-light-emitting surface side of the produced element was sealed by a glass case having a can shape under an ambience of high purity nitrogen gas having a purity of at least 99.999%. The electrode taken out wiring was set to obtain an organic EL element 1-1.

<Preparation of Organic EL Elements 1-2 to 1-31>

Organic EL elements 1-2 to 1-31 were produced in the same manner as the organic EL element 1-1 except that the light-emitting compound was changed as shown in Table I below.

[Evaluation]

<Relative Drive Voltage>

Each of the organic EL elements produced as above was made to emit light at room temperature (about 25° C.) under a constant current of 2.5 mA/cm², and the relative value of the drive voltage (the relative value with respect to the drive voltage of the organic EL element 1-1) was exhibited.

<Relative Brightness Half-Time>

The brightness half-time when each of the above prepared elements was lit at an initial brightness of 300 cd/m² (time required for the brightness to decrease from 300 cd/m² to 150 cd/m²) was measured.

Then, Table I below shows the brightness half-time of each element (relative value with respect to the brightness half-time of the organic EL element 1-1).

TABLE 1

| Organic | | | | Relative | Relative | |
|---|---|---|---|---|---|---|
| EL | Light | | | Drive | Brightness | |
| Element | Emitting | Δest | HOMO | Voltage | half-time | |
| No. | Compound | [eV] | [eV] | [%] | [%] | Remarks |
| 1-1 | Comparative | 0.11 | −4.66 | 100 | 100 | Compar- |
| | Compound 1 | | | | | ative |
| 1-2 | Comparative | 0.15 | −5.41 | 119 | 132 | Compar- |
| | Compound 2 | | | | | ative |
| 1-3 | D-4 | 0.12 | −5.15 | 95 | 167 | Inventive |
| 1-4 | D-6 | 0.11 | −5.08 | 93 | 190 | Inventive |
| 1-5 | D-8 | 0.06 | −4.96 | 91 | 210 | Inventive |
| 1-6 | D-9 | 0.14 | −4.98 | 91 | 165 | Inventive |
| 1-7 | D-16 | 0.07 | −4.99 | 92 | 155 | Inventive |
| 1-8 | D-25 | 0.08 | −5.15 | 94 | 149 | Inventive |
| 1-9 | D-32 | 0.02 | −5.15 | 95 | 142 | Inventive |
| 1-10 | D-33 | 0.08 | −5.09 | 92 | 140 | Inventive |
| 1-12 | D-44 | 0.14 | −5.17 | 93 | 187 | Inventive |
| 1-13 | D-51 | 0.12 | −4.35 | 91 | 199 | Inventive |
| 1-14 | D-56 | 0.10 | −5.12 | 92 | 177 | Inventive |
| 1-15 | D-64 | 0.08 | −5.13 | 92 | 144 | Inventive |
| 1-17 | D-67 | 0.08 | −5.13 | 92 | 142 | Inventive |
| 1-18 | D-75 | 0.15 | −4.91 | 91 | 163 | Inventive |
| 1-19 | D-78 | 0.21 | −4.94 | 91 | 168 | Inventive |
| 1-20 | D-87 | 0.12 | −4.31 | 91 | 175 | Inventive |
| 1-21 | D-91 | 0.15 | −5.14 | 94 | 183 | Inventive |
| 1-22 | D-93 | 0.12 | −4.97 | 91 | 188 | Inventive |
| 1-23 | D-106 | 0.06 | −4.96 | 91 | 240 | Inventive |
| 1-24 | D-109 | 0.08 | −5.14 | 94 | 169 | Inventive |
| 1-25 | D-112 | 0.11 | −5.04 | 94 | 170 | Inventive |
| 1-27 | D-177 | 0.70 | −5.15 | 94 | 158 | Inventive |
| 1-28 | D-134 | 0.03 | −5.16 | 94 | 162 | Inventive |
| 1-29 | D-148 | 0.12 | −4.95 | 91 | 185 | Inventive |
| 1-30 | D-155 | 0.07 | −5.17 | 94 | 148 | Inventive |
| 1-31 | D-177 | 0.10 | −4.29 | 91 | 178 | Inventive |

From the above results, the organic EL element using the compound of the present invention showed lower drive voltage and higher brightness half-time than the organic EL element using the comparative compound.

### Example 3

<Preparation of Organic EL Elements 1-32>

An anode was prepared by making patterning to a glass substrate of 100 mm×100 mm×1.1 mm (NA45, produced by AvanStrate Inc.) on which ITO (indium tin oxide) was formed with a thickness of 100 nm. Thereafter, the above transparent support substrate provided with the ITO transparent electrode was subjected to ultrasonic washing with isopropyl alcohol, followed by drying with desiccated nitrogen gas, and it was subjected to UV ozone washing for 5 minutes.

On the transparent support substrate thus prepared was applied a 70% solution of poly (3,4-ethylenedioxythiphene)-polystyrene sulfonate (PEDOT/PSS, Baytron P AI4083, made by Bayer AG.) diluted with water by using a spin coating method at 3000 rpm for 30 seconds to form a film, and then it was dried at 200° C., for one hour. Thus, a hole injection layer having a thickness of 20 nm was prepared.

Then, a thin film was formed by a spin coating method under the conditions of 2000 rpm and 30 seconds using a solution of polyvinylcarbazole (Mw: about 1100000) in 1,2-dichlorobenzene, and then dried at 120° C. for 10 minutes to form a layer. A hole transport layer having a thickness of 15 nm was provided.

Further, a thin film was prepared by a spin coating method under the conditions of 2000 rpm for 30 seconds using a solution prepared by dissolving the comparative compound 1 as a light-emitting compound. After forming the thin film, it was dried at 100° C. for 10 minutes to provide a light-emitting layer having a layer thickness of 35 nm.

Then, the resulting transparent support substrate was fixed to a substrate holder of a commercial vacuum deposition apparatus.

The constituting materials for each layer were loaded in each crucible for vapor deposition in the vacuum deposition apparatus with an optimum amount. As the crucible for vapor deposition, a crucible made of molybdenum or tungsten made of a resistance heating material was used.

After reducing the pressure to a vacuum degree of 1×10⁻⁴ Pa, SF3-TRZ was vapor-deposited at a vapor deposition rate of 1.0 nm/sec to form a hole blocking layer having a layer thickness of 5 nm.

Then, SF3-TRZ and LiQ (8-hydroxyquinolinolato-lithium) were co-deposited at a vapor deposition rate of 1.0 nm/sec so as to be 50 mol % and 50 mol %, respectively, and an electron transport layer with a layer thickness of 30 nm was formed.

Further, after forming lithium fluoride to a film thickness of 0.5 nm, aluminum was vapor-deposited with layer thickness of 100 nm to form a cathode.

The non-light-emitting surface side of the produced element was sealed by a glass case having a can shape under an ambience of high purity nitrogen gas having a purity of at least 99.999%. The electrode taken out wiring was set to obtain an organic EL element 1-32.

<Preparation of Organic EL Elements 1-33 to 1-36>

Organic EL elements 1-33 to 1-36 were prepared in the same manner as the organic EL element 1-32 except that the light-emitting compound was changed as shown in Table II.

TABLE II

| Organic | | | | Relative | Relative | |
|---|---|---|---|---|---|---|
| EL | Light | | | Drive | Brightness | |
| Element | Emitting | Δest | HOMO | Voltage | half-time | |
| No. | Compound | [eV] | [eV] | [%] | [%] | Remarks |
| 1-32 | Comparative | 0.11 | −4.66 | 100 | 100 | Compar- |
| | Compound 1 | | | | | ative |
| 1-33 | Comparative | 0.15 | −5.41 | 115 | 145 | Compar- |
| | Compound 2 | | | | | ative |
| 1-34 | D-50 | 0.14 | −5.22 | 96 | 190 | Inventive |
| 1-35 | D-406 | 0.06 | −4.96 | 92 | 240 | Inventive |
| 1-36 | D-476 | 0.13 | −5.16 | 93 | 251 | Inventive |

From the above results, the organic EL elements using the compound of the present invention exhibited lower drive voltage and higher brightness half-time than the organic EL element using the comparative compound.

### Example 4

<Preparation of Organic EL Element 1-37>

An anode was prepared by making patterning to a glass substrate of 100 mm×100 mm×1.1 mm (NA45, produced by AvanStrate Inc.) on which ITO (indium tin oxide) was formed with a thickness of 100 nm. Thereafter, the above transparent support substrate provided with the ITO transparent electrode was subjected to ultrasonic washing with isopropyl alcohol, followed by drying with desiccated nitrogen gas, and it was subjected to UV ozone washing for 5 minutes.

On the transparent support substrate thus prepared was applied a 70% solution of poly (3,4-ethylenedioxythiphene)-polystyrene sulfonate (PEDOT/PSS, Baytron P AI4083, made by Bayer AG.) diluted with water by using a spin coating method at 3000 rpm for 30 seconds to form a film, and then it was dried at 200° C. for one hour. Thus, a hole injection layer having a thickness of 20 nm was prepared.

Then, a thin film was formed by a spin coating method under the conditions of 2000 rpm and 30 seconds using a solution of polyvinylcarbazole (Mw: about 1100000) in 1,2-dichlorobenzene, and then dried at 120° C. for 10 minutes to form a layer. A hole transport layer having a thickness of 15 nm was provided.

Then, a solution was prepared by dissolving comparative compound 1 as a light-emitting compound and mCBP as a host compound in propylene glycol monomethyl ether acetate so as to be 10% and 90% by mass, respectively. Using this solution, a piezo type inkjet printer head “KM1024i” manufactured by Konica Minolta, Inc., which is a piezo type inkjet printer head having the structure shown in FIG. 2 described above, was used. According to the manufacturing flow of the organic EL element by the inkjet printing method shown in FIG. 1, the prepared solution was ejected onto the hole transport layer at 40° C. under the condition that the layer thickness after drying was 35 nm, and then dried at 120° C. for 30 minutes. Thus, a light-emitting layer was formed.

Then, the resulting transparent support substrate was fixed to a substrate holder of a commercial vacuum deposition apparatus.

The constituting materials for each layer were loaded in each crucible for vapor deposition in the vacuum deposition apparatus with an optimum amount. As the crucible for vapor deposition, a crucible made of molybdenum or tungsten made of a resistance heating material was used.

After reducing the pressure to a vacuum degree of 1×10⁻⁴ Pa, SF3-TRZ was vapor-deposited at a vapor deposition rate of 1.0 nm/sec to form a hole blocking layer having a layer thickness of 5 nm.

Then, SF3-TRZ and LiQ (8-hydroxyquinolinolato-lithium) were co-deposited at a vapor deposition rate of 1.0 nm/sec so as to be 50 mol % and 50 mol %, respectively, and an electron transport layer with a layer thickness of 30 nm was formed.

Further, after forming lithium fluoride with a film thickness of 0.5 nm, aluminum was vapor-deposited with a layer thickness of 100 nm to form a cathode.

The non-light-emitting surface side of the produced element was sealed by a glass case having a can shape under an ambience of high purity nitrogen gas having a purity of at least 99.999%. The electrode taken out wiring was set to obtain an organic EL element 1-37.

<Preparation of Organic EL Elements 1-14 to I -18>

Organic EL elements 1-38 to 1-41 were produced in the same manner as the organic EL element 1-37 except that the light-emitting compound and the host compound were changed as shown in Table III below.

TABLE III

| Organic | | | | | Relative | Relative | |
|---|---|---|---|---|---|---|---|
| EL | Light | | | | Drive | Brightness | |
| Element | Emitting | Host | Δest | HOMO | Voltage | Half-time | |
| No. | Compound | Compound | [eV] | [eV] | [%] | [%] | Remarks |
| 1-37 | Comparative | mCBP | 0.11 | −4.66 | 100 | 100 | Comparative |
| | Compound 1 | | | | | | |
| 1-38 | Comparative | — | 0.11 | −4.66 | 96 | 21 | Comparative |
| | Compound 1 | | | | | | |
| 1-39 | D-6 | mCBP | 0.11 | −5.08 | 92 | 167 | Inventive |
| 1-40 | D-33 | mCBP | 0.08 | −5.09 | 96 | 132 | Inventive |
| 1-41 | D-30 | — | 0.08 | −5.09 | 94 | 138 | Inventive |

From the above results, the organic EL element using the compound of the present invention showed higher drive voltage and higher brightness half-time than the organic EL element using the comparative compound.

### INDUSTRIAL APPLICABILITY

The present invention is applicable to a π-conjugated compound that has an improved hole injection property causing good element drive voltage and that can reduce aggregates such as exciplex excimers by the π plane-shielded structure, a method for producing the π-conjugated compound, an ink composition, an organic electroluminescent element material, an light emitting material, a charge transport material, a light emitting film, and an organic electroluminescent element.

### REFERENCE SIGNS LIST

1 and 101: Organic EL element

2: Base material

30 and 100: Inkjet head

31 and 39: Pump

32: Filter

33: Piping branch

34: Waste liquid tank

35: Control unit

36, 37, 38A, and 38B: Tank

56: Housing

57: Cap receiving plate

59: Cover member

61: Nozzle plate

62: Cap receiving plate attachment portion

68: Mounting hole

71: Nozzle opening

81a: First joint

81b: Second joint

82: Third joint 102: Glass cover

105: Cathode

106: Organic EL layer

107: Glass substrate with transparent electrode

108: Nitrogen gas

109: Water catching agent

101: Organic EL element

102: Glass cover

105: Cathode

106: Organic EL layer

107: Glass substrate with transparent electrode

108: Nitrogen gas

109: Water catching agent

1. A π-conjugated compound that has a structure of the following Formula (1) and a HOMO level of −5.3 eV or higher,
in the formula, M represents CX1 or a nitrogen atom, X1 to X6 each represent an aromatic substituent or an aromatic heterocyclic group, in which at least four of X1 to X6 are electron-donating condensed aromatic ring substituents with 14 or more π electrons, and at least one of X1 to X6 is an electron-attracting substituent, and X1 to X6 may each independently further have a substituent.

2. The π-conjugated compound according to claim 1, wherein in Formula (1), at least one of the electron-donating condensed aromatic substituents has a structure of any of the following (a) to (c),
in Formulae (a) to (c), R1 to R26 each independently represent a hydrogen atom or a substituent, “#” represents a substitution position to Formula (1), and adjacent substituents may be combined with each other to form a ring structure.

3. The π-conjugated compound according to claim 1, wherein the π-conjugated compound having the structure of Formula (1) has a structure of the following Formula (2),
in the formula, X1 to X6 each represent an aromatic substituent or an aromatic heterocyclic group, in which at least four of X1 to X₆ are electron-donating condensed aromatic ring substituents with 14 or more π electrons, and at least one of X1 to X6 is an electron-attracting substituent, and X1 to X6 may each independently further have a substituent.

4. The π-conjugated compound according to claim 1, wherein in Formula (1) or (2), the HOMO level is −5.0 eV or more.

5. The π-conjugated compound according to claim 1, wherein in Formula (1) or (2), at least one of X1 to X6 has a substituent having a structure of the following Formula (3),
in the formula, a symbol “*” represents a binding position to any of X1 to X6 of Formula (1) or (2), X101 represents NR101, an oxygen atom, a sulfur atom, a sulfinyl group, a sulfonyl group, CR102R103 or SiR104R105, y1 to y8 each independently represent CR106 or a nitrogen atom, R101 to R106 each independently represent a hydrogen atom or a substituent, in which R101 to R106 may be combined with each other to form a ring, n represents an integer of 1 to 4, and R represents a substituent.

6. The π-conjugated compound according to claim 1, wherein an absolute value ΔEst of an energy difference between a lowest excited singlet level and a lowest excited triplet level is 0.50 eV or less.

7. A method for producing the π-conjugated compound according to claim 1,
wherein each of the substituents X1 to X6 is introduced to a benzene ring by a nucleophilic substitution reaction.

8. An ink composition containing the π-conjugated compound according to claim 1.

9. An electroluminescent element material containing the π-conjugated compound according to claim 1.

10. A light emitting material containing the π-conjugated compound according to claim 1,
wherein the π-conjugated compound emits fluorescence.

11. The light emitting material according to claim 10, wherein the π-conjugated compound emits delayed fluorescence.

12. A charge transport material containing the π-conjugated compound according to claim 1,
wherein the π-conjugated compound emits fluorescence.

13. The charge transport material according to claim 12, wherein the π-conjugated compound emits delayed fluorescence.

14. A light emitting film containing the π-conjugated compound according to claim 1.

15. An organic electroluminescent element comprising a pair of electrodes and one or more light-emitting layers,
wherein at least one of the light-emitting layers contains the π-conjugated compound according to claim 1.
